(19) United States
(12) Patent Application Publication
(10) Pub. No.: US 2024/0181044 A1
(43) Pub. Date: Jun. 6, 2024

(54) GLYCAN MODIFIED SPIKE RECEPTOR BINDING DOMAIN NANOPARTICLES AND METHOD OF USE THEREOF AS A CORONAVIRUS DISEASE 2019 (COVID-19) VACCINE

(71) Applicants: Daniel Kulp, Moorestown, NJ (US); Yuanhan Wu, Philadelphia, PA (US); Kylie M. Konrath, Philadelphia, PA (US); Kevin Liaw, Philadelphia, PA (US); Ziyang Xu, Philadelphia, PA (US); David Weiner, Merion, PA (US)

(72) Inventors: Daniel Kulp, Moorestown, NJ (US); Yuanhan Wu, Philadelphia, PA (US); Kylie M. Konrath, Philadelphia, PA (US); Kevin Liaw, Philadelphia, PA (US); Ziyang Xu, Philadelphia, PA (US); David Weiner, Merion, PA (US)

(21) Appl. No.: 18/556,414

(22) PCT Filed: Apr. 20, 2022
(86) PCT No.: PCT/US22/25572

§ 371 Date: Oct. 20, 2023

Related U.S. Application Data
(60) Provisional application No. 63/305,372, filed on Feb. 1, 2022.
(60) Provisional application No. 63/177,361, filed on Apr. 20, 2021.

(51) Int. Cl. A61K 39/215; A61K 9/51; A61K 39/00; A61K 41/00; A61P 31/14; A61P 37/04

(52) U.S. Cl. CPC A61K 39/215; A61K 9/5169; A61K 41/0047; A61P 31/14; A61P 37/04; A61K 2039/6087

(57) ABSTRACT

Disclosed herein are glycan-coated RBD immunogens, engineered nanoparticle vaccines comprising glycan-coated RBD immunogens and methods of use thereof for SARS-COV-2 vaccines.

### CROSS REFERENCE TO RELATED APPLICATIONS

This application claims priority to U.S. Provisional Application No. 63/177,361, filed Apr. 20, 2021, and to U.S. Provisional Application No. 63/305,372, filed Feb. 1, 2022, each of which is hereby incorporated by reference herein in its entirety.

### BACKGROUND

Severe Acute Respiratory Syndrome 2 (SARS-COV-2) virus is responsible for Coronavirus disease 2019 (COVID-19) in over 240 million people and 4.8 million deaths as of Oct. 17, 2021 (Dong et al., 2020, Lancet Infect Dis, 20(5): p. 533-534; Elbe et al., 2017, Glob Chall, 1(1): p. 33-46). The Spike(S) glycoprotein studs the surface of Coronaviruses virions and its receptor-binding domain (RBD) binds host cell receptors to mediate viral entry and infection (Letko et al., 2020, Nat Microbiol, 5(4): p. 562-569; Zhou et al., 2020, Nature, 579(7798): p. 270-273). Greater than 90% of COVID-19 patients produce neutralizing antibodies (nAbs) (Wajnberg et al., 2020, Science, 370(6521): p. 1227-1230) and RBD-directed antibodies often comprise 90% of the total neutralizing response (Piccoli et al., 2020, Cell, 183(4): p. 1024-1042 e21). RBD-directed antibodies can correlate with neutralizing activity (Byrnes et al., 2020 mSphere, 5(5): Suthar et al., 2020, Cell Rep Med, 1(3): p. 100040; Wang et al., 2015, Nat Commun, 6: p. 7712) and ˜2,500 antibodies targeting the SARS-CoV-2 spike have been described to date (Yuan et al., 2020, Biochem Biophys Res Commun, 538: 192-203; Raybould et al., 2020, Bioinformatics). This highlights the importance of eliciting neutralizing antibodies targeting the RBD by vaccination. Rational SARS-COV-2 vaccine design should be informed by spike protein conformation dynamics, the sites of vulnerability and mutations that cause potential vaccine escape. The S trimer has >3,000 residues creating a vast array of epitopes and is targeted by both neutralizing and non-neutralizing antibodies(non-nAbs) (Brouwer et al., 2020, Science, 369(6504): p. 643-650; Ju et al., 2020, Nature, 584(7819): p. 115-119; Liu et al., 2020, Nature, 584(7821): p. 450-456: Rogers et al., 2020, Science, 369(6506): p. 956-963). Measures of RBD binding do not always correlate with neutralization due to presence of non-nAbs, which have the potential to cause antibody-dependent enhancement (Wu et al., 2020, medRxiv 2020 Mar. 30.20047365: Yazici et al., 2020, Journal of Immunology, 205(10): p. 2719-2725; Lee et al., 2020, Nat Microbiol, 5(10): p. 1185-1191). In the context of HIV, influenza and MERS-COV, significant effort over the last few decades has focused on creating immunogens that minimize non-neutralizing epitopes (de Taeye et al., 2018, J Biol Chem, 293(5): p. 1688-1701; Kulp et al., 2017, Nat Commun, 8(1): p. 1655; Sanders et al., 2013, PLOS Pathog, 9(9): p. e1003618: Ren et al., 2016, Curr Opin Immunol, 42: p. 83-90; Impagliazzo et al., 2015, Science, 349(6254): p. 1301-6; Krammer et al., 2013, Curr Opin Virol, 3(5): p. 521-30; Yassine et al., 2015, Nat Med, 21(9): p. 1065-70; Du et al., 2016, Nat Commun, 7: p. 13473). Since the initial outbreak of SARS-COV-2, significant headway has been made in identifying neutralizing epitopes, especially with regards to the RBD; however, study of immunodominant, non-neutralizing epitopes has lagged (Liu et al., 2020, Nature, 584(7821); Barnes et al., 2020, Nature, 588(7839): p. 682-687; Wu et al., 2020, Cell Rep, 33(3): p. 108274). Vaccine immunogens should be developed with these key findings in mind.

Glycosylation is an important post-translational modification in viral pathogenesis serving versatile roles including host cell trafficking and viral protein folding (Watanabe et al., 2019, Biochim Biophys Acta Gen Subj, 1863(10): p. 1480-1497). Mutations introducing potential N-linked glycosylation sites (PNGS) (Hariharan et al., 2020, Biotechnol Bioeng, 117(8): p. 2556-2570) in other viruses such as HIV and influenza have contributed to immune escape (Ly et al., 2000, J Virol, 74(15): p. 6769-76; Medina et al., 2013, Sci Transl Med, 5(187): p. 187ra70; Wanzeck et al., 2011, Am J Respir Crit Care Med, 183(6): p. 767-73; Wei et al., 2003, Nature, 422(6929): p. 307-12). Structure-based vaccine design efforts have been employed to add exogenous PNGS to block non-neutralizing sites and focus the immune response to neutralizing sites (Kulp et al., 2017, Nat Commun, 8(1): p. 1655; Du et al., 2016, Nat Commun, 7: p. 13473; Bajic et al., 2019, Cell Host Microbe, 25(6): p. 827-835 e6; Ingale et al., 2014, J Virol, 88(24): p. 14002-16). These approaches have not yet been widely applied to SARS-COV-2 vaccine development (Shinnakasu et al., 2021, J Exp Med, 218(12)). Here, an advanced structural algorithm was developed for optimizing PNGS into the SARS-COV-2 RBD to focus the immune response and enhance neutralizing responses targeting the Receptor Binding Site epitope (RBS).

Vaccine potency and durability are important for an effective immunological response. Self-assembling, multivalent nanoparticle immunogens (or nanovaccines) enhance the B cell activation and concomitant humoral responses, kinetics of trafficking to the draining lymph nodes and uptake by dendritic cells and macrophages (Xu et al., 2020, Cancer Immunol Res, 8(11): p. 1354-1364; Xu et al., 2020, Adv Sci (Weinh), 7(8): p. 1902802; Manolova et al., 2008, Eur J Immunol, 38(5): p. 1404-13; Kelly et al., 2019, Expert Rev Vaccines, 18(3): p. 269-280). SARS-COV-2 nanovaccines developed as recombinant proteins can be difficult to clinically translate due to arduous purification and manufacturing processes, and further do not tend to activate CD8+ T cells (Xu et al., 2020, Adv Sci (Weinh), 2020. 7(8): p. 1902802).

Accordingly, a need remains in the art for the development of a safe and effective vaccine for the treatment of SARS-COV-2 infection or the treatment or prevention of a disease or disorder associated with SARS-COV-2 infection such as COVID-19.

### SUMMARY OF THE INVENTION

In one embodiment, the invention relates to a nucleic acid molecule encoding a glycan-coated immunogen modified to encode at least one non-native site for glycan modification.

In one embodiment, the nucleotide sequence encoding the immunogen comprises at least two non-native sites for glycan modification.

In one embodiment, the nucleic acid molecule further comprises at least one selected from the group consisting of an IgE leader sequence and a CD4-helper epitope (LS-3).

In one embodiment, the nucleic acid molecule encodes at least two tandem repeats of the immunogen.

In one embodiment, the immunogen is a SARS Coronavirus 2 (SARS-CoV-2) spike protein receptor binding domain (RBD).

In one embodiment, the nucleic acid molecule comprises an expression vector.

In one embodiment, the nucleic acid molecule is incorporated into a self-assembling nanoparticle.

In one embodiment, the invention relates to a nanoparticle comprising a nucleic acid molecule encoding a glycan-coated immunogen modified to encode at least one non-native site for glycan modification. In one embodiment, the nucleotide sequence encoding the immunogen comprises at least two non-native sites for glycan modification. In one embodiment, the nucleic acid molecule further comprises at least one selected from the group consisting of an IgE leader sequence and a CD4-helper epitope (LS-3). In one embodiment, the nucleic acid molecule encodes at least two tandem repeats of the immunogen. In one embodiment, the immunogen is a SARS Coronavirus 2 (SARS-COV-2) spike protein receptor binding domain (RBD).

In one embodiment, the nucleic acid molecule encoding the glycan-coated immunogen is selected from the group consisting of a DNA molecule and an RNA molecule.

In one embodiment, the nucleotide sequence encoding the immunogen modified to encode at least one non-native site for glycan modification is operably linked to a nucleotide sequence encoding a nanoparticle scaffold domain.

In one embodiment, the nanoparticle scaffold domain is IMX313P, ferritin, lumazine synthase, or PcV.

In one embodiment, the nucleic acid molecule comprises a nucleotide sequence encoding a peptide comprising an amino acid sequence having at least about 90% identity over an entire length of an amino acid sequence to SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO: 70, SEQ ID NO:72, SEQ ID NO: 74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO: 100, SEQ ID NO: 102, or SEQ ID NO: 104. In one embodiment, the nucleic acid molecule comprises a nucleotide sequence encoding SEQ ID NO:4, SEQ ID NO:6. SEQ ID NO:8. SEQ ID NO:10. SEQ ID NO: 12. SEQ ID NO: 14. SEQ ID NO:16. SEQ ID NO: 18. SEQ ID NO:20. SEQ ID NO:22. SEQ ID NO:24. SEQ ID NO:26. SEQ ID NO:28. SEQ ID NO:30. SEQ ID NO:32. SEQ ID NO:34. SEQ ID NO:36. SEQ ID NO: 38. SEQ ID NO:40. SEQ ID NO:42. SEQ ID NO:44. SEQ ID NO:46. SEQ ID NO:48. SEQ ID NO:50. SEQ ID NO:52. SEQ ID NO:54. SEQ ID NO:56. SEQ ID NO:58. SEQ ID NO:60. SEQ ID NO:62. SEQ ID NO:64. SEQ ID NO:66. SEQ ID NO: 68. SEQ ID NO: 70. SEQ ID NO: 72. SEQ ID NO:74. SEQ ID NO:76. SEQ ID NO: 78. SEQ ID NO:80. SEQ ID NO:82. SEQ ID NO:84. SEQ ID NO:86. SEQ ID NO:88. SEQ ID NO:90. SEQ ID NO:92. SEQ ID NO:94. SEQ ID NO:96. SEQ ID NO:98. SEQ ID NO: 100. SEQ ID NO: 102, or SEQ ID NO: 104.

In one embodiment, the nucleic acid molecule comprises a nucleotide sequence having at least about 90% identity over an entire length of the nucleic acid to SEQ ID NO:3. SEQ ID NO:5. SEQ ID NO: 7. SEQ ID NO:9. SEQ ID NO: 11. SEQ ID NO:13. SEQ ID NO: 15. SEQ ID NO: 17. SEQ ID NO: 19. SEQ ID NO:21. SEQ ID NO:23. SEQ ID NO:25. SEQ ID NO:27. SEQ ID NO:29. SEQ ID NO:31. SEQ ID NO:33. SEQ ID NO:35. SEQ ID NO:37. SEQ ID NO:39. SEQ ID NO:41. SEQ ID NO:43. SEQ ID NO:45. SEQ ID NO:47. SEQ ID NO:49. SEQ ID NO:51. SEQ ID NO:53. SEQ ID NO:55. SEQ ID NO:57. SEQ ID NO:59. SEQ ID NO:61. SEQ ID NO:63. SEQ ID NO:65. SEQ ID NO: 67. SEQ ID NO:69. SEQ ID NO: 71. SEQ ID NO:74. SEQ ID NO: 75. SEQ ID NO: 77. SEQ ID NO: 79. SEQ ID NO:81. SEQ ID NO:83. SEQ ID NO: 85. SEQ ID NO:87. SEQ ID NO: 89. SEQ ID NO:91. SEQ ID NO:93. SEQ ID NO:95. SEQ ID NO:97. SEQ ID NO:99. SEQ ID NO: 101, or SEQ ID NO:103. In one embodiment, the nucleic acid molecule comprises a nucleotide sequence of SEQ ID NO:3. SEQ ID NO:5. SEQ ID NO:7. SEQ ID NO:9. SEQ ID NO:11. SEQ ID NO: 13. SEQ ID NO: 15. SEQ ID NO: 17. SEQ ID NO: 19. SEQ ID NO:21. SEQ ID NO:23. SEQ ID NO:25. SEQ ID NO:27. SEQ ID NO:29. SEQ ID NO:31. SEQ ID NO:33. SEQ ID NO:35. SEQ ID NO:37. SEQ ID NO:39. SEQ ID NO:41. SEQ ID NO: 43. SEQ ID NO:45. SEQ ID NO:47. SEQ ID NO:49. SEQ ID NO:51. SEQ ID NO:53. SEQ ID NO:55. SEQ ID NO:57. SEQ ID NO:59. SEQ ID NO:61. SEQ ID NO:63. SEQ ID NO:65. SEQ ID NO:67. SEQ ID NO:69. SEQ ID NO:71. SEQ ID NO: 74. SEQ ID NO:75. SEQ ID NO: 77. SEQ ID NO: 79. SEQ ID NO:81. SEQ ID NO: 83. SEQ ID NO:85. SEQ ID NO:87. SEQ ID NO:89. SEQ ID NO:91, SEQ ID NO: 93, SEQ ID NO: 95, SEQ ID NO:97, SEQ ID NO:99, SEQ ID NO:101, or SEQ ID NO:103.

In one embodiment, the invention relates to an immunogenic composition comprising at least one nucleic acid molecule encoding a glycan-coated immunogen modified to encode at least one non-native site for glycan modification or a nanoparticle comprising at least one nucleic acid molecule encoding a glycan-coated immunogen modified to encode at least one non-native site for glycan modification.

In one embodiment, the immunogenic composition, further comprises a pharmaceutically acceptable excipient. In one embodiment, the immunogenic composition further comprises an adjuvant.

In one embodiment, the invention relates to a method of inducing an immune response against SARS-COV-2 in a subject in need thereof, the method comprising administering at least one nucleic acid molecule encoding a glycan-coated immunogen modified to encode at least one non-native site for glycan modification or a nanoparticle comprising at least one nucleic acid molecule encoding a glycan-coated immunogen modified to encode at least one non-native site for glycan modification or an immunogenic composition comprising the same to a subject. In one embodiment, the method of administering includes at least one of electroporation and injection.

In one embodiment, the invention relates to a method of protecting a subject in need thereof from a disease or disorder associate with infection with SARS-CoV-2, the method comprising administering at least one nucleic acid molecule encoding a glycan-coated immunogen modified to encode at least one non-native site for glycan modification or a nanoparticle comprising at least one nucleic acid molecule encoding a glycan-coated immunogen modified to encode at least one non-native site for glycan modification or an immunogenic composition comprising the same to a subject, wherein the subject is thereby resistant to one or more SARS-COV-2 strains or a disease or disorder associated with SARS-COV-2 infection. In one embodiment, the method of administering includes at least one of electroporation and injection.

In one embodiment, the invention relates to a method of treating a subject in need thereof against SARS-COV-2, the method comprising administering at least one nucleic acid molecule encoding a glycan-coated immunogen modified to encode at least one non-native site for glycan modification or a nanoparticle comprising at least one nucleic acid molecule encoding a glycan-coated immunogen modified to encode at least one non-native site for glycan modification or an immunogenic composition comprising the same to a subject. In one embodiment, the method of administering includes at least one of electroporation and injection.

### BRIEF DESCRIPTION OF THE DRAWINGS

FIG. 1A through FIG. 1D depict data demonstrating the CWG algorithm to identify sites amenable to glycosylation. FIG. 1A depicts a diagram of SARS-COV-2 spike trimer (grey) decorated with native glycans (blue) with one RBD in the up state (green) binding to ACE2 (orange) and detailed cartoon representation of RBD with native glycan bound to ACE2. Schematic of wild type glycan distribution across the entire spike. FIG. 1B depicts a CWG pipeline for assessing PNGS on the RBD. FIG. 1C depicts Rosetta scores of glycosylated RBDs for normalized solvent accessible surface (SASA) and residue clash score (fa_rep of sugar residues). FIG. 1C depicts the protein folding (total Rosetta score) vs glycan score (fa_rep of sugar and protein) for each of the glycosylated RBDs selected in (FIG. 1C). Selection criteria shown as dashed lines in (FIG. 1C) and (FIG. 1D).

FIG. 2A through FIG. 2B depicts data demonstrating the modeling and survey of native glycans on human virus proteins. FIG. 2A depicts data demonstrating that native glycans were modeled on PNGS using the modified Rosetta GlycanTreeModeler script on four human viral glycoproteins: envelope of HIV, hemagglutinin of H1N1, and Spike proteins of SARS-COV and MERS-COV. FIG. 2B depicts data demonstrating the repulsive glycan energy of individual modeled glycans on each native PNGS sites were surveyed and a cut-off value of 5.0 (REU) is used to include all possible native glycosylation scenarios.

FIG. 3A through FIG. 3E depict data demonstrating the in vitro characterization of single glycan variants of RBD. FIG. 3A depicts a model of selected glycan sites (blue spheres) on the RBD (green cartoon) interacting with ACE2 binding helices (orange cartoon). FIG. 3B depicts a small scale screen of selected variants binding to ACE2 in Area Under the Curve from ELISA binding curves and normalized to WT binding (bars), qualitative expression from Western Blot represented as +/− symbols above the bars. FIG. 3C depicts neutralizing epitopes mapped on RBD structure with RBD in grey surface and ACE2 binding helices in orange, surface patches are color according to: RBD-A,B,C are in orange; RBD-D in red; RBD-E in green, RBD-F in blue. FIG. 3D depicts SPR binding kinetics of single glycan variants to a panel of SARS-COV-2 antibodies. FIG. 3E depicts the relative binding as measured by ELISA EC50 ratio of glycan variants binding to WT RBD binding in a panel of neutralizing and non-neutralizing antibodies. Blue to Red coloring was done based on stronger or weaker binding relative to WT RBD.

FIG. 4 depicts a table of the kinetic constants for RBD-antibody interactions modeled well by 1:1 Langmuir fitting.

FIG. 5A through FIG. 5G depicts exemplary experimental data demonstrating the in vitro and in vivo antigenic profile of multiglycan RBDs. FIG. 5A depicts data demonstrating the surface representation of RBD (green) bound to ACE2 (orange cartoons) with glycans (red sticks) for the WT RBD and RBD g5.1 constructs. Designed glycan voxels, or the space sampled by glycans, are depicted as purple blocks FIG. 5B depicts data demonstrating the relative binding as measured by ELISA EC50 ratio of glycan variants binding to a panel of neutralizing and non-neutralizing antibodies to WT RBD binding. Blue to Red coloring was done based on stronger or weaker binding relative to WT RBD. FIG. 5C depicts data demonstrating the antibody binding titers and FIG. 5D depicts data demonstrating the pseudovirus ID50 neutralization titers from BALB/c mice immunized with 25 μg of plasmids encoding WT RBD or RBD g5.1 at week 0 and 2. FIG. 5E depicts data demonstrating the ACE2 competition assay layout for measuring blocking of ACE2 interacting with RBD with RBS-directed antibodies from the sera of vaccinated mice. FIG. 5E depicts data demonstrating the fraction of ACE2 binding blocked by antibodies in ACE2 competition assay and FIG. 5F depicts data demonstrating the blocking titer measured as the first dilution of sera at which a reduction in ACE2 binding is observed. (unpaired two tailed Student t-test (FIG. 5F) p=0.0020, (FIG. 5F) p=0.0236).

FIG. 6A through FIG. 6E depicts the rationale for generating glycan combinations. FIG. 6A depicts a distance map of all residue pairs on native RBD to approximate distances between two engineered glycans. Distance between the geometric center of each residue of any residue pair on RBD is calculated using Py Rosetta script. The distance information is subsequently visualized as a Distance Heatmap in R. Only glycans that are 10-20 Angstroms away from each other can be selected for a combination. FIG. 6B depicts an example of distance measurements for a combination of three glycan additions where the residue of added glycans (red spheres) is on RBD (green) bound to two helices of ACE2 (orange) with one native glycan (blue). Distances between each engineered glycan is shown in black dash and labeled with the distance value in Angstroms. FIG. 6C depicts a table summary of all combinations made in this study with glycan addition positions and distance between engineered glycans. Glycan compositions at each potential N-linked glycan site (PNGS) are represented as bar graphs present in RBD g5.1 monomer (FIG. 6D) and RBD g8.2 monomer (FIG. 6E). Glycans were categorized and colored according to the detected compositions. Oligomannose-type glycans (M9-M4) are colored green. Hybrid-type glycans, those containing three HexNAcs and at least five hexoses, were colored as for complex-type glycans because one arm can be processed in a similar manner. Complex-type glycans were categorized according to the number of HexNAc residues detected and the presence or absence of fucose. Core glycans represent any detected composition smaller than HexNAc₂Hex₃. For hybrid and complex-type glycans, bars are colored to represent the terminal processing present. Blue represents agalactosylated, yellow galactosylated (containing at least one galactose), and purple sialylated (containing at least one sialic acid). The proportion of unoccupied PNGSs is colored gray.

FIG. 7A through FIG. 7D depicts data demonstrating the prime-boost RBD and RBDg5.1 immunogenicity. FIG. 7A depicts antibody binding titers and FIG. 7B depicts pseudovirus ID50 neutralization titers from BALB/c mice immunized with 25 μg of plasmids encoding WT RBD or RBD g5.1 at week 0) and 2. FIG. 7C depicts the fraction of ACE2 binding blocked by antibodies in ACE2 competition assay and FIG. 7D depicts the blocking titer measured as the first dilution of sera at which a reduction in ACE2 binding is observed. (unpaired two tailed Student t-test (FIG. 7C) p=0.0020, (FIG. 7D) p=0.0236).

FIG. 8A through FIG. 8I depict data demonstrating the immune focused RBD nanoparticle structure and immunogenicity. FIG. 8A depicts models of 8 different RBD nanovaccines. In each model, the coloring is as follows: RBS (yellow) on the RBD (green) coated with glycans (blue) fused with a glycine-serine linker (gray) to a nanoparticle scaffold (red). FIG. 8B depicts endpoint titers for a single BALB/c mouse immunized with once with 2 μg of plasmid encoding RBD nanoparticles by DNA-E.P, colored as indicated on the figure, in vitro expression and assembly of nanoparticles indicated in the ‘ASM’ column as either expressed/assembled (A), poor expression/assembly (X) or not tested (N). FIG. 8C depicts size-exclusion chromatogram (curves indicate UV absorbance and correspond to the left y-axis) and multiangle light scattering data of RBD g5.1 multimers (black line under each curve indicates molecular weight and correspond to the right y-axis). FIG. 8D depicts the 2D class averages showing RBDs decorating the RBD g5.1 24mer. FIG. 8E depicts the cryo-EM density map of RBD g5.1 24mer at low threshold, the 24mer scaffold could be unambiguously determined (FIG. 17), the flexible linker attachment points for the RBDs on the 24mer scaffold could be observed at low density threshold (blue dots) (FIG. 8F). Glycan compositions at each potential N-linked glycan site (PNGS) are represented as bar graphs present in RBD g5.1 24-mer. Glycans were categorized and colored according to the detected compositions. Oligomannose-type glycans (M9-M4) are colored green. Hybrid-type glycans, those containing three HexNAcs and at least five hexoses, were colored as for complex-type glycans because one arm can be processed in a similar manner. Complex-type glycans were categorized according to the number of HexNAc residues detected and the presence or absence of fucose. Core glycans represent any detected composition smaller than HexNAc₂Hex₃. For hybrid and complex-type glycans, bars are colored to represent the terminal processing present. Blue represents agalactosylated, yellow galactosylated (containing at least one galactose), and purple sialylated (containing at least one sialic acid). The proportion of unoccupied PNGSs is colored gray. FIG. 8G depicts endpoint titers for expanded groups (n=5) of BALB/c mice immunized with 2 μg of plasmid encoding RBD nanoparticles by DNA-E.P. FIG. 8H depicts pseudovirus neutralization of SARS-CoV-2 variants B.1(WT), B.1.351(Beta), B.1.1.7(Alpha), P.1(Gamma) and B.1.617.2(Delta) by sera from BALB/c mice immunized with 5 μg RBD g5.1 24mer. (I) Pseudovirus neutralization of SARS-COV-2 variants B.1(WT), B.1.351(Beta), B.1.1.7(Alpha), P.1(Gamma) and B.1.617.2(Delta) over time.

FIG. 9A through FIG. 9E depict data demonstrating that WT RBD nanoparticles and pseudovirus neutralization of expanded groups (n=5) in BALB/c mice. FIG. 9A depicts size exclusion chromatograms for WT RBD nanoparticles. FIG. 9B depicts endpoint titers from binding ELISA to RBD of immunizations with 2 μg of DNA-launched WT RBD nanoparticles. FIG. 9C depicts pseudovirus neutralization of BALB/c mice immunized (n=5 or 10) with 2 μg immune focused DNA-launched nanoparticles. * p<0.05 (Two-way ANOVAs vs. RBD: RBD g8.2 7mer p=0.0111, RBD g5.1 24mer p=0.0205, RBD g8.2 24mer p=0.0199, RBD g8.2 60mer p=0.0135). FIG. 9D and FIG. 9E depict endpoint binding titer (FIG. 9D) and neutralization titer (FIG. 9E) against SARS-COV-2 and variants of concern for sera from BALB/c mice immunized with PI. RBD g5.1 24mer.

FIG. 10 depicts a cryo-EM of RBD g5.1 24mer immunogen. Cryo-EM data processing followed standard routines. 3D reconstruction was performed under assumption of octahedral symmetry as well as asymmetrically. The two resulting density maps demonstrate flexible linker attachment points at low density threshold at identical places. Final resolutions were 3.4 Å and 3.9 Å, respectively as can be confirmed by visual inspection of the density close-ups. Density for RBDs is disordered due to the inherent flexible linker in the immunogen design.

FIG. 11A through FIG. 11F depict exemplary experimental data demonstrating the immunogenicity of RBD nanovaccines in C57BL/6 mice. Endpoint titers (FIG. 11A) and pseudo virus neutralization ID50s (FIG. 11B) for C57BL/6 mice immunized with 1 μg or 5 μg of four selected RBD nanovaccines. FIG. 11C depicts data demonstrating a IFN-γ ELISpot assay with splenocytes from mice immunized with RBD monomer, RBD g5.1 24mer, and RBD g5.1 120mer vaccines, or the naive. Intracellular staining of IFN-γ (FIG. 11D), surface staining of CD107a (FIG. 11E), and intracellular staining of TNFα (FIG. 11F) of effector memory CD8+ CD44+ CD62− T cells from splenocytes. Error bars indicate means±SD (n=3−5 mice/group). Splenocytes were stimulated by native RBD peptides in FIG. 11C—FIG. 11F. ((FIG. 11C) unpaired two-tailed Student t-tests vs. naïve: RBD monomer p=0.0063, RBD g5.1 24mer p=0.0049, RBD g5.1 120mer 1 μg p<0.0019, RBD g5.1 120mer 5 μg p<0.0001). ((FIG. 11D) unpaired two-tailed Student t-tests vs. naïve: RBD monomer p<0.0001, RBD g5.1 24mer p=0.0010, RBD g5.1 120mer 1 μg p=0.0005, RBD g5.1 120mer 5 μg p<0.0001; vs. RBD Monomer: RBD g5.1 120mer 5 μg p=0.0123). ((FIG. 11E) unpaired two-tailed Student t-tests vs. naïve: RBD monomer p=0.0007, RBD g5.1 24mer p=0.0018, RBD g5.1 120mer 1 μg p=0.0031, RBD g5.1 120mer 5 μg p<0.0001: vs. RBD Monomer: RBD g5.1 120mer 5 μg p=0.0063). ((FIG. 11F) unpaired two-tailed Student t-tests vs. naïve: RBD monomer p=0.0013, RBD g5.1 24mer p=0.0153, RBD g5.1 120mer 1 μg p=0.0038, RBD g5.1 120mer 5 μg p=0.0002).

FIG. 12 depicts data demonstrating protein nanoparticle immunization. Endpoint titers of BALB/c mice immunized SC with 10 μg of RBD g8.2 7mer and 24mer protein co-formulated with RIBI adjuvant.

FIG. 13A through FIG. 13C depicts data demonstrating emerging variants of concern. FIG. 13A depicts endpoint titers of BALB/c mice immunized with 5 μg of RBD g5.1 24mer (WT RBD) binding to WT, B. 1.351(Beta), B.1.1.7(Alpha), and P.1(Gamma) RBDs and individual mutation RBDs. FIG. 13B depicts SEC trace of P.1/Gamma RBD g5.1 24mer. FIG. 13C depicts a comparison of neutralization of BALB/c mice immunized with RBD monomer 2 μg and Spike 10 μg against variant pseudoviruses.

FIG. 14A through FIG. 14G depicts data demonstrating lethal challenge of SARS-COV-2 in rodent model. FIG. 14A depicts a K18 hACE2 lethal challenge study overview. FIG. 14B depicts data demonstrating SARS-COV-2 live virus neutralization one day prior to challenge. **** p<0.0001. FIG. 14C depicts weight loss of K18 hACE2 mice after SARS-COV-2 challenge. FIG. 14D depicts Kaplan-Meier curves representing survival of K18 hACE2 mice after SARS-COV-2 challenge. (Mantel-Cox test vs. naïve: RBD monomer p=0.0327, RBD g5.1 24mer p=0.0006, RBD g5.1 120mer 1 μg p=0.0087, RBD g5.1 120mer 5 μg p=0.0006; vs. RBD monomer: RBD g5.1 120mer 5 μg p=0.0426, RBD g5.1 24mer p=0.0048). FIG. 14E depicts pseudovirus neutralization titers of surviving and non-surviving mice. (unpaired two-tailed Student t-test p=0.0003). FIG. 14F depicts the correlation between body weight change at day 4 post-challenge and pre-challenge live virus neutralizing titers (ID₅₀). FIG. 14G depicts viral titers in nasal turbinates, brain and lung tissue at day 4 post challenge. (unpaired two-tailed Student t-test vs. naïve: RBD 120mer 1 μg p=0.0110, RBD 120mer 5 μg p=0.0109, RBD g5.1 24mer p=0.0110). LOD for this assay (lower dashed line) is lower than the LOD reported elsewhere (top dashed line).

FIG. 15 depicts data demonstrating the pre-challenge pseudo virus neutralization of K18 hACE2 mice. Pseudo virus neutralization of week 3 sera from K18 hACE2 mice immunized for challenge study.

FIG. 16A through FIG. 16H depicts data demonstrating the humoral responses to nanovaccines in OmniMouse®, Guinea Pigs and Hamsters. FIG. 16A depicts data demonstrating the human antibody titers from OmniMouse® immunized three times four weeks apart with 25 μg of DNA encoding RBD nanoparticles as measured by combined AUC from ELISA curves with human IgK and human IgL secondaries. FIG. 16B depicts data demonstrating the human antibody titers from OmniMouse® immunized three times four weeks apart with 25 μg of DNA encoding RBD nanoparticles as measured by endpoint titer using human IgK and human IgL secondaries. FIG. 16C depicts the pseudo virus neutralization titers at week 6 and week 8 post immunization. FIG. 16D depicts the endpoint titers against RBD for sera from Hartley guinea pigs immunized with RBD monomer and RBD g5.1 24mer after a single dose. FIG. 16E depicts the pseudo virus neutralization of sera from guinea pigs immunized with RBD monomer and RBD g5.1 24mer after a single dose (unpaired t-test vs. naïve: 5 μg RBD g5. 1 24mer week 2 p=0.0140, week 3 p=0.0003, week 4 p=0.0146: 10 μg RBD g5. 1 24mer: wk 2 p-0.0145, week 3 p=0.0016, week 4 p=0.0007. Unpaired t-test vs 10 μg RBD to 5 μg RBD g5. 1 24mer week 2 p=0.0142, week 3 p=0.0104; 10 μg RBD g5. 1 24mer week 2 p=0.0002, week 3 p=0.0042, week 4 p=0.0304. FIG. 16F depicts the endpoint binding titers against RBD for sera from Syrain Golden hamsters immunized with RBD monomer or RBD 48mer two times with two different doses. FIG. 16G depicts the neutralization of SARS-COV-2 pseudovirus by sera from hamsters immunized with RBD monomer or RBD 48mer with two different doses. (unpaired two-tailed Student t-test: RBD 48mer 10 μg vs. RBD monomer 10 μg p=0.0079, RBD 48mer 2 μg vs. RBD monomer 2 μg p=0.0004). FIG. 16H depicts lung lavages from hamsters immunized with RBD monomer or RBD 48mer.

FIG. 17A through FIG. 17C depicts data demonstrating the additional Omni mice serology. FIG. 17A depicts AUC of binding ELISAs from Omni mice immunized with DNA-launched RBD nanoparticles with mIgG, rIgM, and rIgG breakdown. FIG. 17B depicts AUC of binding ELISAs from Omni mice with hIgL and hIgK breakdown. FIG. 17C depicts murine leukemia virus (MLV) neutralization of Omni mice immunized with DNA-launched RBD nanoparticles demonstrate no nonspecific neutralization.

FIG. 18 depicts data demonstrating that RBD nanos can be engineered to induce neutralizing antibodies against highly diverse variants of concern (VOCs) including Omicron and BA2.

### DETAILED DESCRIPTION OF THE INVENTION

The present invention relates to the development of glycan-coated RBD immunogens and the engineering of eight multivalent configurations. Advanced DNA delivery of the engineered nanoparticle vaccines rapidly elicited potent neutralizing antibodies in guinea pigs, hamsters and four mouse models, including human ACE2 and human B cell repertoire transgenics. RBD nanoparticles encoding wild-type, B.135, B.1.7.1 and P.1 SARS-COV-2 variants induced cross-neutralizing antibodies. Single, low dose immunization protected against a lethal SARS-COV-2 challenge. Single-dose coronavirus vaccines via DNA-launched nanoparticles provide a platform for rapid clinical translation of novel, potent coronavirus vaccines.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′)₂, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a full length wild type strain SARS-COV-2 antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a SARS-COV-2 protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. GLYCAN-COATED IMMUNOGENS

The invention is based in part on the development of glycan-coated immunogen nanoparticles and nucleic acid molecules encoding the same, wherein the nucleic acid molecules comprise a nucleotide sequence encoding an immunogen modified to comprise at least one non-native site for glycan modification, linked to a nucleotide sequence encoding a nanoparticle scaffold domain. In one embodiment, the nucleotide sequence encoding the glycan-coated immunogen nanoparticle further comprises a leader sequence, a CD4-helper epitope (LS-3), or a combination thereof.

In one embodiment, the nucleotide sequence encoding the glycan-coated immunogen nanoparticle comprises at least two tandem repeats of the immunogen sequences. In one embodiment, the immunogen is a SARS Coronavirus 2 (SARS-CoV-2) spike protein receptor binding domain (RBD).

### SARS Coronavirus 2 (SARS-COV-2) Antigen

As described above, in one embodiment, the invention relates to a vaccine comprising a SARS-COV-2 antigen, a fragment thereof, a variant thereof, or a combination thereof. Coronaviruses, including SARS-COV-2, are encapsulated by a membrane and have a type 1 membrane glycoprotein known as spike (S) protein, which forms protruding spikes on the surface of the coronavirus. The spike protein facilitates binding of the coronavirus to proteins located on the surface of a cell, for example, the metalloprotease amino peptidase N, and mediates cell-viral membrane fusion. In particular, the spike protein contains an SI subunit that facilitates binding of the coronavirus to cell surface proteins and thus comprises a receptor binding domain (RBD). Accordingly, the SI subunit of the spike protein controls which cells are infected by the coronavirus. In one embodiment, the SARS-COV-2 antigen of the invention can comprise one or more SARS-COV-2 spike protein RBD.

The SARS-COV-2 antigen can be a SARS-COV-2 spike protein RBD, a fragment thereof, a variant thereof, or a combination thereof. In one embodiment, the composition of the invention comprises a dimer of the SARS-COV-2 spike protein RBD.

In one embodiment, the composition of the invention is capable of eliciting an immune response in a mammal against one or more SARS-COV-2 strains. The SARS-COV-2 antigen can comprise an epitope(s) that makes it particularly effective as an immunogen against which an anti-SARS-COV-2 immune response can be induced.

Exemplary SARS-COV-2 coronavirus strains include, but are not limited to, an Alpha (B.1.1.7 and Q lineages) strain or variant thereof, a Beta (B.1.351 and descendent lineages) strain or variant thereof, a Delta (B.1.617.2 and AY lineages) strain or variant thereof, an Epsilon (B.1.427 and B.1.429) strain or variant thereof, a Gamma (P.1 and descendent lineages) strain or variant thereof, an Eta (B.1.525) strain or variant thereof, an Iota (B.1.526) strain or variant thereof, a Kappa (B.1.617.1) strain or variant thereof, a Mu (B.1.621, B.1.621.1) strain or variant thereof, an Omicron (B.1.1.529 and BA lineages) strain or variant thereof or a Zeta (P.2) strain or variant thereof, or any combination thereof.

The SARS-COV-2 spike protein RBD can be a consensus sequence derived from two or more strains of SARS-COV-2. The SARS-COV-2 spike antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the one or more SARS-COV-2 spike protein RBD. The one or more SARS-COV-2 spike protein RBD can comprise a signal peptide such as an immunoglobulin signal peptide, for example, but not limited to, an immunoglobulin E (IgE) or immunoglobulin (IgG) signal peptide.

The SARS-COV-2 RBD can have an amino acid sequence of SEQ ID NO:2. In some embodiments, the SARS-COV-2 RBD can be an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2.

The nucleic acid molecule encoding the SARS-COV-2 RBD antigen can comprise the nucleic acid sequence of SEQ ID NO:1, which encodes SEQ ID NO:2. In some embodiments, the nucleic acid molecule encoding the SARS-COV-2 RBD antigen can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2. In some embodiments, the nucleic acid molecule encoding the SARS-COV-2 RBD antigen can comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO: 1. In some embodiments, the SARS-COV-2 RBD antigen can be operably linked to an IgE leader sequence.

Immunogenic fragments of SEQ ID NO:2 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:2 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% identity to SEQ ID NO:2. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1. Immunogenic fragments can be at least 60%, at least 65%, at least 70%, at least 75%, at least 80%, at least 85%, at least 90%, at least 95%, at least 96%, at least 97%, at least 98% or at least 99% of the full length of SEQ ID NO: 1. Immunogenic fragments can comprise at least 95%, at least 96%, at least 97% at least 98% or at least 99% identity to fragments of SEQ ID NO:1. In some embodiments, immunogenic fragments include sequences that encode a leader sequence, such as for example an immunoglobulin leader, such as the IgE leader. In some embodiments, fragments are free of coding sequences that encode a leader sequence.

### Glycan Modification Sites

In some embodiments, the nucleic acid molecule encoding the SARS-CoV-2 antigen of the invention further encodes at least one non-natural glycan modification site. In some embodiments, the nucleic acid molecule encoding the SARS-CoV-2 further encodes at least 2, 3, 4, 5, 6, 7, 8, or more than 8 non-natural glycan modification sites.

In some embodiments, the invention relates to a composition comprising a glycan-modified SARS-COV-2 RBD antigen. In one embodiment, the glycan-modified SARS-COV-2 RBD antigen comprises at least 1, 2, 3, 4, 5, 6, 7, 8, or more than 8 non-natural glycan. In one embodiment, the nucleotide sequence encoding the glycan-modified SARS-COV-2 RBD antigen comprises at least 1, 2, 3, 4, 5, 6, 7, 8, or more than 8 mutations that provide non-natural glycan modification sites on the encoded protein.

In one embodiment, the glycan-modified SARS-COV-2 RBD antigen comprises amino acids 331-527 of the SARS-COV-2 spike glycoprotein (PDB: 6M0J) comprising at least 1, 2, 3, 4, 5, 6, 7, 8, or more than 8 non-natural glycan modification sites. In one embodiment, the nucleic acid molecule encoding the glycan-modified SARS-COV-2 RBD antigen comprises a nucleotide sequence encoding amino acids 331-527 of the SARS-COV-2 spike glycoprotein (PDB: 6M0J) comprising at least 1, 2, 3, 4, 5, 6, 7, 8, or more than 8 mutations that promote non-natural glycan modification of the RBD antigen.

### SARS-COV-2 Spike RBD Mutimer

The SARS-COV-2 antigen can be a multimer of the SARS-COV-2 RBD antigen, a fragment thereof, or a variant thereof. The multimeric SARS-COV-2 RBD antigen may comprise at least 2, at least 3, at least 4 or more than 4 copies of the SARS-CoV-2 RBD. The multimeric SARS-COV-2 RBD antigen can comprise a linker sequence between the two or more RBD antigens. The multimeric SARS-COV-2 RBD antigen can comprise a consensus sequence and/or modification(s) for improved expression. Modification can include codon optimization, RNA optimization, addition of a kozak sequence for increased translation initiation, and/or the addition of an immunoglobulin leader sequence to increase the immunogenicity of the outlier SARS-CoV-2 spike antigen. The multimeric SARS-COV-2 RBD antigen can comprise a signal peptide such as an immunoglobulin signal peptide, for example, but not limited to, an immunoglobulin E (IgE) or immunoglobulin (IgG) signal peptide. The dimeric SARS-CoV-2 RBD antigen can be designed to elicit a stronger cellular and/or humoral immune response than a monomeric SARS-COV-2 RBD antigen.

The multimeric SARS-COV-2 RBD antigen can comprise at least two RBD sequences. In one embodiment, the nucleic acid molecule encoding the multimeric SARS-COV-2 RBD antigen encodes at least two copies of the same SARS-COV-2 RBD sequence. In some embodiments, the nucleic acid molecule encoding the multimeric SARS-COV-2 RBD antigen encodes at least two copies of SEQ ID NO:2. In some embodiments, the nucleic acid molecule encoding the multimeric SARS-COV-2 RBD antigen comprises at least two copies of SEQ ID NO: 1. In one embodiment, the nucleic acid molecule encoding the multimeric SARS-COV-2 RBD antigen encodes at least two different SARS-COV-2 RBD sequences. In one embodiment, the nucleic acid molecule encoding the multimeric SARS-COV-2 RBD antigen encodes the amino acid sequence SEQ ID NO:2 and at least one additional nucleotide sequence that encodes an amino acid sequence having at least about 90%, 91%, 92%, 93%, 94%, 95%, 96%, 97%, 98%, 99%, or 100% identity over an entire length of the amino acid sequence set forth in SEQ ID NO:2. In some embodiments, the nucleic acid molecule encoding the multimeric SARS-COV-2 RBD antigen comprises SEQ ID NO: 1 and at least one additional nucleotide sequence having at least about 90%, 91%, 92%, 93%, 94%, 95%, 96%, 97%, 98%, 99% or 100% identity over an entire length of the nucleic acid sequence set forth in SEQ ID NO: 1. In some embodiments, the multimeric SARS-COV-2 RBD antigen can be operably linked to an IgE leader sequence.

Immunogenic fragments of SEQ ID NO:2 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:2 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:2. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:1. Immunogenic fragments can be at least 95%, at least 96%, at least 97% at least 98% or at least 99% homologous to fragments of SEQ ID NO:1. In some embodiments, immunogenic fragments include sequences that encode a leader sequence, such as for example an immunoglobulin leader, such as the IgE leader. In some embodiments, fragments are free of coding sequences that encode a leader sequence.

In some embodiments, the nucleic acid molecule encoding the multimeric SARS-COV-2 RBD antigen of the invention further encodes at least one non-natural glycan modification site. In some embodiments, the nucleic acid molecule encoding the multimeric SARS-COV-2 RBD antigen further encodes at least 2, 3, 4, 5, 6, 7, 8, or more than 8 non-natural glycan modification sites.

In some embodiments, the invention relates to a composition comprising a glycan-modified multimeric SARS-COV-2 RBD antigen. In one embodiment, the glycan-modified multimeric SARS-COV-2 RBD antigen comprises at least 1, 2, 3, 4, 5, 6, 7, 8, or more than 8 non-natural glycan. In one embodiment, the nucleotide sequence encoding the glycan-modified multimeric SARS-COV-2 RBD antigen comprises at least 1, 2, 3, 4, 5, 6, 7, 8, or more than 8 mutations that provide non-natural glycan modification sites on the encoded protein.

In one embodiment, the glycan-modified multimeric SARS-COV-2 RBD antigen comprises amino acids 331-527 of the SARS-COV-2 spike glycoprotein (PDB: 6M0J) comprising at least 1, 2, 3, 4, 5, 6, 7, 8, or more than 8 non-natural glycan modification sites. In one embodiment, the nucleic acid molecule encoding the glycan-modified multimeric SARS-COV-2 RBD antigen comprises a nucleotide sequence encoding amino acids 331-527 of the SARS-COV-2 spike glycoprotein (PDB: 6M0J) comprising at least 1, 2, 3, 4, 5, 6, 7, 8, or more than 8 mutations that promote non-natural glycan modification of the multimeric SARS-COV-2 RBD antigen.

### Self-Assembling Nanoparticles

In one embodiment, one or more SARS-COV-2 antigen is incorporated into a self-assembling peptide nanoparticle (SAPN). Self-assembling protein nanoparticles (SAPN) may be formed by the assembly of one or more polypeptide chains comprising at least one antigen and at least one protein oligomerization domain. Without limitation, the SAPN of the invention may self-assemble into a tetrahedron, a cube, an octahedron, a dodecahedron, or an icosahedron. The SAPN of the invention may be used as an efficient means for presenting one or more SARS-COV-2 antigen.

In one embodiment, the SAPN of the invention comprises the receptor binding domain of the SARS-COV-2 spike protein. In one embodiment, the SAPN of the invention comprises a dimer of the receptor binding domain of the SARS-COV-2 spike protein.

In one embodiment, the invention relates to a self-assembling nanoparticle comprising an oligomerization domain and further comprising a SARS-CoV-2 antigen, a fragment thereof, a variant thereof, or a combination thereof.

In one embodiment, the invention relates to a nucleic acid molecule encoding a glycan-coated SARS-COV-2 Spike RBD self-assembling nanoparticle, a fragment thereof, a variant thereof, or a combination thereof. In some embodiments, the nucleic acid molecule encoding the glycan-coated SARS-COV-2 Spike RBD self-assembling nanoparticle can comprise a nucleotide sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO: 70, SEQ ID NO: 72, SEQ ID NO: 74, SEQ ID NO: 76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO: 100, SEQ ID NO: 102, or SEQ ID NO:104, or a fragment or variant thereof.

Immunogenic fragments of SEQ ID NO:4, SEQ ID NO:6, SEQ ID NO:8, SEQ ID NO: 10, SEQ ID NO: 12, SEQ ID NO: 14, SEQ ID NO: 16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO: 40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO: 62, SEQ ID NO:64, SEQ ID NO:66, SEQ ID NO:68, SEQ ID NO: 70, SEQ ID NO: 72, SEQ ID NO: 74, SEQ ID NO: 76, SEQ ID NO:78, SEQ ID NO:80, SEQ ID NO:82, SEQ ID NO:84, SEQ ID NO:86, SEQ ID NO:88. SEQ ID NO:90, SEQ ID NO:92, SEQ ID NO:94, SEQ ID NO:96, SEQ ID NO:98, SEQ ID NO: 100, SEQ ID NO: 102, or SEQ ID NO: 104 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:4, SEQ ID NO:6, SEQ ID NO:8, SEQ ID NO:10, SEQ ID NO: 12, SEQ ID NO: 14, SEQ ID NO: 16, SEQ ID NO: 18, SEQ ID NO:20, SEQ ID NO: 22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO: 62, SEQ ID NO: 64, SEQ ID NO:66, SEQ ID NO:68, SEQ ID NO:70, SEQ ID NO: 72, SEQ ID NO: 74, SEQ ID NO: 76, SEQ ID NO:78, SEQ ID NO:80, SEQ ID NO: 82, SEQ ID NO:84, SEQ ID NO:86, SEQ ID NO:88, SEQ ID NO:90, SEQ ID NO: 92, SEQ ID NO:94, SEQ ID NO:96, SEQ ID NO:98, SEQ ID NO:100, SEQ ID NO: 102, or SEQ ID NO: 104.

In one embodiment, the invention relates to a nucleic acid molecule encoding a glycan-coated SARS-COV-2 Spike RBD self-assembling nanoparticle, a fragment thereof, a variant thereof, or a combination thereof. In some embodiments, the nucleic acid molecule encoding the glycan-coated SARS-COV-2 Spike RBD self-assembling nanoparticle can comprise a nucleotide sequence that encodes the amino acid sequence having at least about 80%, 81%, 82%, 83%, 84%, 85%, 86%, 87%, 88%. 89%, 90%, 91%, 92%, 93%, 94%, 95%, 96%, 97%, 98%, 99%, or 100% identity over an entire length of the amino acid sequence set forth in SEQ ID NO:3. SEQ ID NO:5. SEQ ID NO:7. SEQ ID NO:9. SEQ ID NO:11. SEQ ID NO: 13. SEQ ID NO:15. SEQ ID NO: 17. SEQ ID NO: 19. SEQ ID NO:21. SEQ ID NO:23. SEQ ID NO:25. SEQ ID NO:27. SEQ ID NO: 29. SEQ ID NO:31. SEQ ID NO:33. SEQ ID NO:35. SEQ ID NO:37. SEQ ID NO: 39. SEQ ID NO:41. SEQ ID NO:43. SEQ ID NO:45. SEQ ID NO:47. SEQ ID NO:49. SEQ ID NO:51. SEQ ID NO:53. SEQ ID NO:55. SEQ ID NO:57. SEQ ID NO:59. SEQ ID NO:61. SEQ ID NO:63. SEQ ID NO:65. SEQ ID NO:67. SEQ ID NO: 69. SEQ ID NO: 71. SEQ ID NO: 74. SEQ ID NO: 75. SEQ ID NO:77. SEQ ID NO: 79. SEQ ID NO:81. SEQ ID NO:83. SEQ ID NO:85. SEQ ID NO:87. SEQ ID NO: 89. SEQ ID NO:91. SEQ ID NO:93. SEQ ID NO:95. SEQ ID NO:97. SEQ ID NO:99. SEQ ID NO:101, or SEQ ID NO: 103. In some embodiments, the nucleic acid molecule encoding the RBD-NP can comprise a nucleotide sequence having at least about 80%, 81%, 82%, 83%, 84%, 85%, 86%, 87%, 88%, 89%, 90%. 91%, 92%, 93%, 94%, 95%, 96%, 97%, 98%, 99% or 100% identity over an entire length of the nucleic acid sequence set forth in SEQ ID NO:3. SEQ ID NO:5. SEQ ID NO:7. SEQ ID NO:9. SEQ ID NO: 11. SEQ ID NO: 13. SEQ ID NO: 15. SEQ ID NO: 17. SEQ ID NO: 19. SEQ ID NO:21. SEQ ID NO:23. SEQ ID NO:25. SEQ ID NO: 27. SEQ ID NO:29. SEQ ID NO:31. SEQ ID NO:33. SEQ ID NO:35. SEQ ID NO:37. SEQ ID NO:39. SEQ ID NO:41. SEQ ID NO:43. SEQ ID NO:45. SEQ ID NO:47. SEQ ID NO:49. SEQ ID NO: 51. SEQ ID NO:53. SEQ ID NO:55. SEQ ID NO:57. SEQ ID NO:59. SEQ ID NO:61. SEQ ID NO:63. SEQ ID NO:65. SEQ ID NO:67. SEQ ID NO:69. SEQ ID NO: 71. SEQ ID NO: 74, SEQ ID NO: 75. SEQ ID NO:77. SEQ ID NO:79. SEQ ID NO:81. SEQ ID NO:83. SEQ ID NO:85. SEQ ID NO:87. SEQ ID NO:89. SEQ ID NO: 91. SEQ ID NO: 93. SEQ ID NO: 95. SEQ ID NO:97. SEQ ID NO:99. SEQ ID NO: 101, or SEQ ID NO: 103.

Immunogenic fragments of SEQ ID NO:3. SEQ ID NO:5. SEQ ID NO:7. SEQ ID NO:9. SEQ ID NO:11. SEQ ID NO: 13. SEQ ID NO: 15. SEQ ID NO:17. SEQ ID NO: 19. SEQ ID NO:21. SEQ ID NO:23. SEQ ID NO:25. SEQ ID NO:27. SEQ ID NO:29. SEQ ID NO:31. SEQ ID NO:33. SEQ ID NO:35. SEQ ID NO:37. SEQ ID NO:39. SEQ ID NO:41. SEQ ID NO:43. SEQ ID NO:45. SEQ ID NO:47. SEQ ID NO:49. SEQ ID NO:51. SEQ ID NO:53. SEQ ID NO:55. SEQ ID NO:57. SEQ ID NO:59, SEQ ID NO:61, SEQ ID NO:63, SEQ ID NO:65, SEQ ID NO:67, SEQ ID NO:69, SEQ ID NO:71, SEQ ID NO: 74, SEQ ID NO:75, SEQ ID NO: 77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, or SEQ ID NO:103 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:3, SEQ ID NO:5, SEQ ID NO: 7, SEQ ID NO:9, SEQ ID NO: 11, SEQ ID NO: 13, SEQ ID NO: 15, SEQ ID NO: 17, SEQ ID NO: 19, SEQ ID NO:21, SEQ ID NO:23, SEQ ID NO: 25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO: 65, SEQ ID NO:67, SEQ ID NO: 69, SEQ ID NO: 71, SEQ ID NO:74, SEQ ID NO: 75, SEQ ID NO: 77, SEQ ID NO: 79, SEQ ID NO:81, SEQ ID NO:83, SEQ ID NO: 85, SEQ ID NO: 87, SEQ ID NO:89, SEQ ID NO:91, SEQ ID NO:93, SEQ ID NO:95, SEQ ID NO:97, SEQ ID NO:99, SEQ ID NO: 101, or SEQ ID NO:103.

### Leader Sequence

In some embodiments, the glycan-coated SARS-COV-2 Spike RBD self-assembling nanoparticle sequences of the invention are operably linked to at least one leader sequence or a pharmaceutically acceptable salt thereof. In some embodiments, the nucleic acid molecules of the invention encoding the glycan-coated SARS-COV-2 Spike RBD self-assembling nanoparticle sequences are operably linked to at least one nucleotide sequence encoding a leader sequence or a pharmaceutically acceptable salt thereof. “Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

In one embodiment, the leader sequence is the IgE leader sequence comprising the amino acid sequence of MDWTWILFLVAAATRVHS (SEQ ID NO: 105). In one embodiment, the fragments of the glycan-coated SARS-COV-2 Spike RBD self-assembling nanoparticle sequences of the invention are lacking the leader sequence of SEQ ID NO:105.

### Linker Sequence

In some embodiments, the glycan-coated SARS-COV-2 Spike RBD self-assembling nanoparticle sequences of the invention are operably linked to at least one linker sequence. A linker can be either flexible or rigid or a combination thereof. In one embodiment, the linker is a (GGS)ₙ repeat wherein, the GGS is repeated at least 1, 2, 3, 4, 5, 6, 7, 8, 9, or 10 or more than 10 times. In some embodiments, the expressible nucleic acid sequence comprises at least one nucleic acid sequence encoding an LS3 linker comprising the amino acid sequence of LRFGIVASRANHALVGGSGG (SEQ ID NO: 106). In some embodiments, the at least one nucleic acid sequence, encoding a linker, encodes a sequence comprising at least about 70%, 75%, 80%, 85%, 90%, 95%, or 99% sequence identity to SEQ ID NO: 106 or a pharmaceutically acceptable salt thereof.

In one embodiment, the fragments of the glycan-coated SARS-COV-2 Spike RBD self-assembling nanoparticle sequences of the invention are lacking the linker sequence of SEQ ID NO:106.

### Vector

In one embodiment, the invention relates to one or more vectors that include a nucleic acid encoding the glycan-coated SARS-COV-2 Spike RBD self-assembling nanoparticle. The one or more vectors can be capable of expressing the glycan-coated SARS-COV-2 Spike RBD self-assembling nanoparticle. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### (1) Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### (2) Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

### (3) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### Immunogenic Composition

Provided herein are immunogenic compositions, such as vaccines, comprising a glycan-coated SARS-COV-2 Spike RBD self-assembling nanoparticle sequence, a fragment thereof, a variant thereof, or a combination thereof. The immunogenic composition can be used to treat SARS-COV-2 infection, thereby treating, preventing, and/or protecting against SARS-COV-2 based pathologies. In one embodiment, the SARS-COV-2 based pathology is COVID-19. The vaccine can significantly induce an immune response of a subject administered the vaccine, thereby protecting against and treating SARS-COV-2 infection.

The immunogenic composition can be a DNA vaccine, an RNA vaccine, a peptide vaccine, or a combination thereof. The immunogenic composition can include a nucleic acid molecule comprising a sequence encoding the SARS-COV-2 antigen in the form of a self-assembling nanoparticle. The nucleic acid sequence can be DNA, RNA, cDNA, a variant thereof, a fragment thereof, or a combination thereof. The nucleic acid sequence can also include additional sequences that encode linker, leader, or tag sequences that are linked to the SARS-COV-2 antigen by a peptide bond.

The immunogenic composition can induce a humoral immune response in the subject administered the immunogenic composition. The induced humoral immune response can be specific for the SARS-COV-2 antigen. The induced humoral immune response can be reactive with the SARS-COV-2 antigen. The humoral immune response can be induced in the subject administered the immunogenic composition by about 1.5-fold to about 16-fold, about 2-fold to about 12-fold, or about 3-fold to about 10-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the immunogenic composition can include an increased level of neutralizing antibodies associated with the subject administered the immunogenic composition as compared to a subject not administered the immunogenic composition. The neutralizing antibodies can be specific for the SARS-COV-2 antigen. The neutralizing antibodies can be reactive with the SARS-COV-2 antigen. The neutralizing antibodies can provide protection against and/or treatment of SARS-COV-2 infection and its associated pathologies in the subject administered the immunogenic composition.

The humoral immune response induced by the immunogenic composition can include an increased level of IgG antibodies associated with the subject administered the immunogenic composition as compared to a subject not administered the immunogenic composition. These IgG antibodies can be specific for the SARS-COV-2 antigen. These IgG antibodies can be reactive with the SARS-COV-2 antigen. In one embodiment, the humoral response is cross-reactive against two or more strains of the SARS-COV-2.

For example, in one embodiment, the humoral response is cross-reactive against one or more SARS-COV-2 coronavirus strain including, but not limited to, an Alpha (B.1.1.7 and Q lineages) strain or variant thereof, a Beta (B.1.351 and descendent lineages) strain or variant thereof, a Delta (B.1.617.2 and AY lineages) strain or variant thereof, an Epsilon (B.1.427 and B.1.429) strain or variant thereof, a Gamma (P.1 and descendent lineages) strain or variant thereof, an Eta (B.1.525) strain or variant thereof, an Iota (B.1.526) strain or variant thereof, a Kappa (B.1.617.1) strain or variant thereof, a Mu (B.1.621, B.1.621.1) strain or variant thereof, an Omicron (B.1.1.529 and BA lineages) strain or variant thereof or a Zeta (P.2) strain or variant thereof, or any combination thereof.

The level of IgG antibody associated with the subject administered the immunogenic composition can be increased by about 1.5-fold to about 16-fold, about 2-fold to about 12-fold, or about 3-fold to about 10-fold as compared to the subject not administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the immunogenic composition.

The immunogenic composition can induce a cellular immune response in the subject administered the immunogenic composition. The induced cellular immune response can be specific for the SARS-COV-2 antigen. The induced cellular immune response can be reactive to the SARS-COV-2 antigen. Preferably, the cellular response is cross-reactive against two or more strains of the SARS-COV-2.

For example, in one embodiment, the cellular response is cross-reactive against one or more SARS-COV-2 coronavirus strain including, but not limited to, an Alpha (B.1.1.7 and Q lineages) strain or variant thereof, a Beta (B.1.351 and descendent lineages) strain or variant thereof, a Delta (B.1.617.2 and AY lineages) strain or variant thereof, an Epsilon (B.1.427 and B.1.429) strain or variant thereof, a Gamma (P.1 and descendent lineages) strain or variant thereof, an Eta (B.1.525) strain or variant thereof, an Iota (B.1.526) strain or variant thereof, a Kappa (B.1.617.1) strain or variant thereof, a Mu (B.1.621, B.1.621.1) strain or variant thereof, an Omicron (B.1.1.529 and BA lineages) strain or variant thereof or a Zeta (P.2) strain or variant thereof, or any combination thereof.

The induced cellular immune response can include eliciting a CD8⁺ T cell response. The elicited CD8⁺ T cell response can be reactive with the SARS-COV-2 antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the immunogenic composition as compared to the subject not administered the immunogenic composition. The CD8⁺ T cell response associated with the subject administered the immunogenic composition can be increased by about 2-fold to about 30-fold, about 3-fold to about 25-fold, or about 4-fold to about 20-fold as compared to the subject not administered the immunogenic composition. The CD8⁺ T cell response associated with the subject administered the immunogenic composition can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the immunogenic composition.

The induced cellular immune response can include an increased frequency of CD3⁺CD8⁺ T cells that produce IFN-γ. The frequency of CD3⁺CD8⁺IFN-γ⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the immunogenic composition.

The induced cellular immune response can include an increased frequency of CD3⁺CD8⁺ T cells that produce TNF-α. The frequency of CD3⁺CD8⁺TNF-α⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the immunogenic composition.

The induced cellular immune response can include an increased frequency of CD3⁺CD8⁺ T cells that produce IL-2. The frequency of CD3⁺CD8⁺IL-2⁺ T cells associated with the subject administered the immunogenic composition can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the immunogenic composition.

The induced cellular immune response can include an increased frequency of CD3⁺CD8⁺ T cells that produce both IFN-γ and TNF-α. The frequency of CD3⁺CD8⁺IFN-γ⁺TNF-α⁺ T cells associated with the subject administered the immunogenic composition can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the immunogenic composition.

The cellular immune response induced by the immunogenic composition can include eliciting a CD4⁺ T cell response. The elicited CD4⁺ T cell response can be reactive with the SARS-COV-2 antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the immunogenic composition.

The induced cellular immune response can include an increased frequency of CD3⁺CD4⁺ T cells that produce TNF-α. The frequency of CD3⁺CD4⁺TNF-α⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the immunogenic composition.

The induced cellular immune response can include an increased frequency of CD3⁺CD4⁺ T cells that produce IL-2. The frequency of CD3⁺CD4⁺IL-2⁺ T cells associated with the subject administered the immunogenic composition can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the immunogenic composition.

The induced cellular immune response can include an increased frequency of CD3⁺CD4⁺ T cells that produce both IFN-γ and TNF-α. The frequency of CD3⁺CD4⁺IFN-γ⁺TNF-α⁺ associated with the subject administered the immunogenic composition can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the immunogenic composition.

The immunogenic composition of the present invention can have features required of effective vaccines such as being safe so the immunogenic composition itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The immunogenic composition can further induce an immune response when administered to different tissues such as the muscle or skin. The immunogenic composition can further induce an immune response when administered via electroporation, or injection, or subcutaneously, or intramuscularly.

### Excipients and Other Components of the Immunogenic Composition

A composition comprising a glycan-coated immunogen of the invention, or nucleic acid molecule encoding the same (e.g., a vaccine of the invention), may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in an alternative plasmid or are delivered as proteins in combination with the plasmid above in the vaccine. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Ser. No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 3. METHOD OF VACCINATION

Also provided herein is a method of treating, protecting against, and/or preventing disease in a subject in need thereof by administering the glycan-coated immunogens of the invention to the subject or nucleic acid molecules encoding the same. Administration of the glycan-coated immunogens, or nucleic acid molecules encoding the same, of the invention to the subject can induce or elicit an immune response in the subject. The induced immune response can be used to treat, prevent, and/or protect against disease, for example, pathologies relating to SARS-COV-2 infection. In one embodiment, the pathology relating to SARS-COV-2 infection is COVID-19.

The induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the SARS-COV-2 spike RBD. The induced cellular immune response can include a CD8⁺ T cell response, which is induced by about 2-fold to about 30-fold, about 3-fold to about 25-fold, or about 4-fold to about 20-fold.

The dose of the glycan-coated immunogens of the invention, or nucleic acid molecules encoding the same, can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### Administration

The glycan-coated immunogens of the invention, or nucleic acid molecules encoding the same, can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The glycan-coated immunogens of the invention, or nucleic acid molecules encoding the same, can be administered prophylactically or therapeutically. In prophylactic administration, the glycan-coated immunogens of the invention, or nucleic acid molecules encoding the same, can be administered in an amount sufficient to induce an immune response. In therapeutic applications, the glycan-coated immunogens of the invention, or nucleic acid molecules encoding the same, are administered to a subject in need thereof in an amount sufficient to elicit a therapeutic effect. An amount adequate to accomplish this is defined as “therapeutically effective dose.” Amounts effective for this use will depend on, e.g., the particular composition of the therapeutic regimen administered, the manner of administration, the stage and severity of the disease, the general state of health of the patient, and the judgment of the prescribing physician.

The glycan-coated immunogens of the invention, or nucleic acid molecules encoding the same, can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. In some embodiments, DNA molecules can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the glycan-coated immunogens of the invention, or nucleic acid molecules encoding the same.

The glycan-coated immunogens of the invention, or nucleic acid molecules encoding the same, can be delivered via a variety of routes. Typical delivery routes include parenteral administration, e.g., intradermal, intramuscular or subcutaneous delivery. Other routes include oral administration, intranasal, and intravaginal routes. In some embodiments, nucleic acid molecules can be delivered to the interstitial spaces of tissues of an individual (Felgner et al., U.S. Pat. Nos. 5,580,859 and 5,703,055, the contents of all of which are incorporated herein by reference in their entirety). The glycan-coated immunogens of the invention, or nucleic acid molecules encoding the same, can also be administered to muscle, or can be administered via intradermal or subcutaneous injections, or transdermally, such as by iontophoresis. Epidermal administration of the glycan-coated immunogens of the invention, or nucleic acid molecules encoding the same, can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The glycan-coated immunogens of the invention, or nucleic acid molecules encoding the same, can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the glycan-coated immunogens of the invention, or nucleic acid molecules encoding the same.

The glycan-coated immunogens of the invention, or nucleic acid molecules encoding the same, can be a liquid preparation such as a suspension, syrup or elixir. The glycan-coated immunogens of the invention, or nucleic acid molecules encoding the same, can also be a preparation for parenteral, subcutaneous, intradermal, intramuscular or intravenous administration (e.g., injectable administration), such as a sterile suspension or emulsion.

The glycan-coated immunogens of the invention, or nucleic acid molecules encoding the same, can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The glycan-coated immunogens of the invention, or nucleic acid molecules encoding the same, can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the glycan-coated immunogens of the invention, or nucleic acid molecules encoding the same, and associated fluid gradually while the needle is being inserted leads to a more even distribution of the fluid through the body tissue. The pain experienced during injection may be reduced due to the distribution of the glycan-coated immunogens of the invention, or nucleic acid molecules encoding the same, being injected over a larger area.

The MID may inject the glycan-coated immunogens of the invention, or nucleic acid molecules encoding the same, into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell PA) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided. A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### 4. KIT

Provided herein is a kit, which can be used for treating a subject using the method of vaccination described above. In one embodiment, the kit can comprise the vaccine. In one embodiment, the kit can comprise a nucleic acid molecule encoding a glycan-coated SARS-COV-2 Spike RBD self-assembling nanoparticle of the invention.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, links to websites, QR codes, electronic storage media (e.g., magnetic discs, tapes, cartridges), optical media (e.g., CD ROM), and the like. As used herein, the term “instructions” can include the address of an internet site which provides instructions.

The present invention has multiple aspects, illustrated by the following non-limiting examples.

### 5. EXAMPLES

### Example 1: Nucleic Acid Delivery of Immune-Focused SARS-COV-2 Nanoparticles Drive Rapid and Potent Immunogenicity Capable of Single-Dose Protection

New SARS-COV-2 vaccines should (1) alleviate cold chain requirements for global vaccine distribution, (2) improve immunogenicity in the elderly and immunocompromised populations, (3) have increased efficacy when administered as a single dose or as a booster and (4) protect against emerging variants that reduce or evade current vaccine-induced immunity. The data presented herein demonstrate that advanced DNA formulation and delivery technology coupled with immune focused nanovaccines can provide a platform to address these translational obstacles for SARS-CoV-2 vaccines.

Viral glycan evolution results in antigenic changes with concomitant immune evasion. It has been observed that for influenza, humoral immunity becomes restricted over time due to glycan additions (Altman et al., 2019, mBio, 10(2)). SARS-CoV-2 mutational variants may escape from antibody-mediated immunity. Glycan mutational variants may begin to circulate given their large impact on antibody recognition of virus. Here, a map of possible glycan additions to the RBD of SARS-CoV-2 and their effect on a large series nAbs is provided. Interestingly, it was found that a glycan at position 458, 369, 450 and 441 can bind to human ACE2, but strongly reduces binding to nAbs targeting the sites RBD-A, B, C and D respectively. Further, the glycan occupancy is an important parameter for immunogen design, as underoccupancy at glycan site has been shown to elicit non-neutralizing antibodies (Cottrell et al., 2020, PLOS Pathog, 16(8): p. e1008753: Derking et al., 2021, Cell Rep, 35(1): p. 108933). Addition of glycans to RBD-based nanovaccines can improve expression, assembly, immunogenicity and neutralization. One potential limitation of this approach is that while the addition of glycans to vaccine immunogens can prevent induction of non-neutralizing antibodies and enrich for neutralizing antibodies, glycans may also alter other antibody binding which may be important. As with all in vivo antigen generating platforms including DNA, tissue expression may have effects on glycan structure due to differences in enzymatic availability. Additional studies following up on glycosylation differences of in vivo and in vitro immunogens are warranted.

The synthetic DNA platform employed in this study can be leveraged for generation of enhanced immunity, easier global distribution and rapid reformulation. New adaptive electroporation systems can improve uptake of DNA plasmid up to 500 times (Gary et al., 2020, Curr Opin Immunol, 65: p. 21-27). In stark contrast to complex recombinant protein and RNA-based product development, DNA-based production and purification are extremely easy due to availability of off the shelf commercial purification kits used widely in research laboratories. DNA vaccines are also much more chemically and thermally stable allowing storage at room temperature for long periods of time. These characteristics of the DNA platform allows for new vaccines to be developed at breakneck speed and distributed to resource limited settings around the globe.

A key finding in this study is the dose-sparing immunogenicity afforded by the nanoparticle designs. Most SARS-COV-2 vaccines require at least two doses (Polack et al., 2020, N Engl J Med, 383(27): p. 2603-2615; Baden et al., 2021, N Engl J Med, 384(5): p. 403-416). DNA vaccines often require higher vaccine doses (25 μg in mice (Smith et al., 2020, Nat Commun, 11(1): p. 2601) and 5 mg in NHPs (Yu et al., 2020, Science, 369(6505): p. 806-811) and/or advanced delivery devices to drive sufficient immunogenicity. Here, strong immunogenicity and protection was observed from bona fide SARS-COV-2 challenge down to 1 μg. However, weight loss was observed in 2/6 mice after challenge and low viral titers in 2/4 mice sacrificed four days after challenge. Increasing the dose to 10 μg or by prime-boosting could improve on these results. In fact, studies of DNA-launched nanoparticle vaccines in guinea pigs and hamsters demonstrated greater immunogenicity than RBD monomer at a low dose of 10 μg.

In comparison to other RBD nanoparticle systems, significant improvements have been demonstrated. Recently, studies on two-component spike-based nanoparticle showed strong immunogenicity with sporadic pseudovirus neutralization 2 weeks post prime (Brouwer et al., 2021, Cell, 184(5): p. 1188-1200 e19; Walls et al., 2020, Cell, 183(5): p. 1367-1382 e17). After 2 doses of the i53-50 RBD nanoparticle vaccine, mice challenged with 1×10⁵ PFU of a mouse-adapted non-lethal virus were observed to have reduced viral replication (Walls et al., 2020, Cell, 183(5): p. 1367-1382 e17). Spy Tag-coupled RBD nanoparticles induce binding but not neutralizing antibodies 2-weeks post prime (Cohen et al., 2021, Science, 371(6530): p. 735-741; He et al., 2021, bioRxiv, 2020.09.14.296715; Zhang et al., 2020, Sci Rep, 10(1): p. 18149; Tan et al., 2021, Nat Commun, 12(1): p. 542). An RBD-HR Spy Tag nanoparticle was observed to induce immunity after 2 doses which after challenge with 4×10⁴ PFU authentic SARS-COV-2 could reduce viral load in the lungs (Ma et al., 2020, Immunity, 53(6): p. 1315-1330 e9). Here, the DNA-launched glycan modified RBDs could be genetically fused with four different nanoparticles scaffolds, the simple genetic fusion results in a single vaccine product that could induce binding and neutralizing antibodies 1 week post prime immunization and induce CD8+ T cells. A more stringent test of immunity was created as compared to most previous studies as authentic SARS-CoV-2 virus with 2.5-fold higher amount of virus (1×10⁵ PFU) was used in the challenge along with a 10-fold more sensitive viral detection assay. Further, vaccines studied in this model mostly utilize a prime and boost to achieve protection (personal communication, Texas Biomed). In this model, the nanovaccines could induce immunity that reduced viral replication and completely protected from death at a low single dose of 5 μg. From the data, there is an 82% chance of survival if mice have a live virus neutralization titer >100 prior to challenge. Further there were high levels of cross-reactivity neutralization to the B.1.1.7(Alpha), B.1.351(Beta), P. 1(Gamma) and B.1.617.2(Delta) SARS-COV-2 variants generated by the nanovaccine which protected in lethal challenge. In addition, the P.1/Gamma RBD g5.1 nanoparticle elicited high levels of cross-reactive antibodies which could be employed as a booster vaccine.

Thus, the data presented herein demonstrate that a single-dose SARS-CoV-2 nanovaccine has been developed with a platform that can afford rapid preclinical reconfiguration to address variants of concern and for clinical translation.

### Methods

Cloaking with Glycans Algorithm

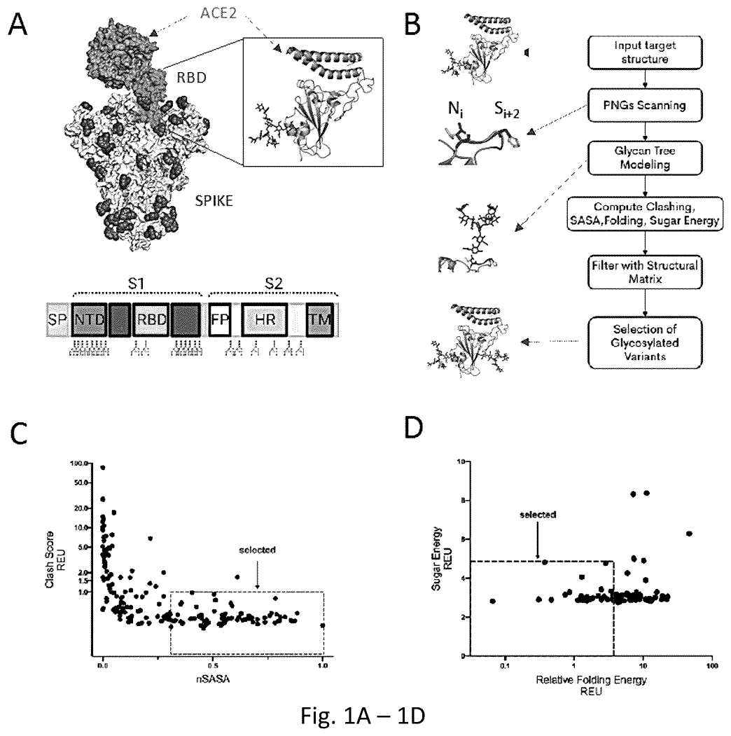

The modeling started with RBD structure PDB id: 6M0J. GlycanTreeModeler(GTM) is a glycan modeling algorithm recently developed in Rosetta(unpublished). The Cloaking With Gycans (CWG) workflow utilizes GTM for selecting single glycan addition positions on target protein. All steps in CWG are summarized in a flowchart (FIG. 1B). CWG begins with detecting native sequons and modeling all the native glycan structures using Man9GlcNAc2 glycans on the target protein. In the next stage, a model is made for the addition of a single glycan at each position. A given position in the protein is mutated to asparagine and the i+2 position is mutated into threonine or serine. The model with the lowest energy i+2 position is used for further evaluation. The Rosetta energy is computed for the resulting model. Positions were filtered out if the total energy of the model corresponding to that position had a total energy >5 Rosetta Energy Units (REU) more than the native structure. Next, the CWG algorithm builds Man9GlcNAc2 glycans on the mutated position and measures repulsive energy of engineered glycan between sugar-sugar and sugar-protein energy terms. Some positions were filtered out based on structural criteria, such as avoiding the mutation of positions involved in disulfide bonds. Man9GlcNAc2 glycans were utilized for simplicity.

### Nanoparticle Modeling

All nanoparticles were modeled with corresponding designed structures and linkers. Four nanoparticles were used in this study: IMX313P (PDB id: 4B0F), ferritin (PDB id: 3BVE), lumazine synthase (PDB id: 1HQK), and PcV (PDB id: 3J3I). Biological unit nanoparticle structure files were downloaded in CIF format. The termini of the monomeric RBDs were aligned to the termini of the nanoparticle, rotational and translational degrees of freedom were sampled to reduce clashing between RBDs and nanoparticles, extended linkers of various lengths were then aligned to fuse the nanoparticle and immunogen with simpleNanoparticleModeling from the MSL library as previously described (Xu et al., 2020, Adv Sci (Weinh), 7(8): p. 1902802).

### Protein Expression and Purification

Glycosylated RBDs: A gene encoding the amino acids 331-527 of the SARS-COV-2 spike glycoprotein (PDB: 6M0J) was mutated at each position according to CWG. Nanoparticles were genetically fused to designed RBDs as described above. DNA encoding the variants were codon optimized for Homo sapiens and cloned with a IgE secretion sequence into the pVAX vector. A 6×HisTag was added to the c-terminus of the RBD monomer variants. ExpiF293 cells were transfected with the pVAX plasmid vector either carrying the nanoparticles or the His-Tagged monomer transgene with PEI/Opti-MEM and harvested 6-7 days post transfection. The supernatants was first purified with affinity chromatography using the AKTA pure 25 purification system and IMAC Nickel column (HisTrap™ HP prepacked Column, Cytiva) for His-tagged monomers and gravity flow columns filled with Agarose bound Galnthus Nivalis Lectin beads (Vector Labs) for nanoparticles. The eluate fractions from the affinity chromatography were pooled, concentrated, and dialyzed into 1×PBS before being loaded onto the Size-Exclusion Chromatography (SEC) column for further purification with Superdex 200 Increase 10/300 GL column for the His-tagged monomers and the Superose 6 Increases 10/300 GL column for the nanoparticles. Fractions of interest were pooled and concentrated for characterization. For antibody production, heavy and light chains were encoded in pFUSEss-CHIg-hG1, and pFUSE2ss-CLIg-hk or pFUSEss-CLIg-hL2 respectively and were co-transfected in equal parts using ExpiFectamine™ 293 Transfection Kit(Gibco) according to manufacturer's protocol. Antibodies were purified by affinity chromatography using the Protein A column (HiTrap™ MabSelect™ SuRe, Cytiva) and AKTA Pure 25 purification system.

### Western Blot

Samples were prepared with 13 μL supernatants of Expi293F cells transfected with RBD monomer plasmids or 0.65 μg of purified WT RBD in 1×PBS, NuPAGE LDS Sample Buffer (Novex), and NuPAGE Sample Reducing Agent (Novex) were denatured at 90° C. for 10 minutes. Samples were loaded in a 4-12% SDS Bis-Tris gel for electrophoresis then transferred from the gel onto a PVDF membrane. The membrane was blocked with Intercept (PBS) Blocking Buffer (LI-COR) for >1 hour at ambient temperature then incubated with *** μg/protein gel of MonoRab anti-his tag C-term (Genscript) in Intercept T20 (PBS) Antibody Diluent (LI-COR) overnight at 4° C. The membrane was then incubated in a 1:10000 IRDye 800CW goat anti-rabbit IgG (LI-COR Biosciences) in Intercept T20 (PBS) Antibody Diluent (LI-COR) at room temperature for 1 h. Membranes were imaged with a LI-COR Odyssey CLx.

### ELISA

For in vitro characterization, high Binding, 96-well Flat-Bottom, Half-Area Microplate (Corning) were coated at 1 μg/mL 6×-His tag polyclonal antibody (Invitrogen) for >4 hours at ambient temperature and blocked ≥1 hour with 5% milk/1×PBS/0.01% Tween-20 at 4° C. RBD transfection supernatant or recombinant protein at μg/mL was incubated for 1-2 hours at ambient temperature. Serial dilutions of antibodies were made according to affinity and incubated on plate for 1-2 hours at ambient temperature. Goat anti-Human IgG-Fe fragment cross-adsorbed antibody HRP conjugated (Bethyl Laboratories) secondary at a 1:10,000 dilution for 1 hour at ambient temperature. All dilutions except coating were performed in 5% milk/1×PBS/0.01% Tween-20 and plates were washed with 1×PBS/0.05% Tween-20 between steps. 1-Step™ Ultra TMB-ELISA Substrate Solution (Thermo Scientific) was incubated on the plate for 10 minutes in the dark and then quenched with 1 M H2SO4. Absorbance of samples at 570 nm was subtracted from 450 nm for each well and background of blank wells were subtracted from each well before analysis. Curves were analyzed in GraphPad Prism 8 with Sigmoidal, 4PL, X is concentration and AUC.

For serology, plates were coated with 1 μg/mL 6×-His tag polyclonal antibody (Invitrogen) in 1×PBS for 6 hours at ambient temperature and blocked overnight with 0.5% NCS/5% Goat Serum/5% Milk/0.2% PBS-T. 5× serial dilutions of sera were made starting at a 1:100 dilution and incubated on plate for 2 hours at 37° C. For BL6, BALB/c, and K18 ACE2 mouse studies, goat anti-mouse IgG h+1 HRP-tagged antibody (Bethyl Laboratories) diluted 1:20000. For the OmniMouse® study, Peroxidase AffiniPure Goat Anti-Rat IgG (Jackson ImmunoResearch) at 1:10000, Peroxidase AffiniPure F(ab′)₂ Fragment Goat Anti-Rat IgM, u chain specific (Jackson ImmunoResearch) at 1:10000, Goat anti-Human Kappa Light Chain Antibody HRP Conjugated (Bethyl Laboratories) at 1:10000, Goat anti-Human Lambda Light Chain Antibody HRP Conjugated (Bethyl Laboratories) at 1:10000, and Goat anti-Mouse IgG-heavy and light chain Antibody HRP Conjugated (Bethyl Laboratories) at 1:20000, and Goat anti-guinea pig IgG whole molecule (Sigma) at 1:10,000 were used. Secondary antibodies were incubated on plates for 1 hr at RT. All dilutions except coating were performed in 1% NCS in 0.2% PBS-T and plates were washed with 1×PBS/0.05% Tween-20 between steps. Plates were developed with 1 Step Ultra TMB substrate in the dark for 10 minutes for mouse studies and 15 minutes for guinea pig studies before being quenched with 1N H₂SO₄ and read using a BioTek Synergy 2 plate reader at an absorbance of 450 and 570 nm.

Hamster serology was performed by directly coating 96-well flat bottom, half-area plates #3690 (Corning) with 25 mL of 1 μg/mL of SARS-COV-2 RBD (University of Texas, Austin) overnight at 4° C. Plates were blocked with 100 μL of blocking buffer (3% BSA in 1×PBS) for 1 hr at 37° C. Hamster sera was diluted to 1:16 dilution in diluent buffer (1% BSA in PBS) and an 11-point 1:3 serial dilution was done on the ELISA plate, with last column containing only dilution buffer as blank control. ELISA plates were incubated for 2 hr at 37° C. with sera dilutions. Anti-Hamster HRP antibody (Sigma) was diluted in diluent buffer 1:10,000 and were incubated for 1 hr at room temperature. SureBlue TMB 1-Component Microwell Peroxidase Substrate (KPL) was added to the wells and plates were incubated for 6 minutes and then quenched with TMB Stop Solution (KPL). Absorbance was immediately read at 450 nm on Synergy HTX plate reader (BioTek). All volumes except blocking buffer was 25 μL. Plates were washed 3 times with wash buffer (0.05% Tween 20 in 1×PBS) between steps.

### Surface Plasmon Resonance

RBD-antibody kinetics experiments were performed with a Series S Sensor Protein A capture chip (Cytiva) on a Biacore 8k instrument (GE). The running buffer was HBS-EP (3 M sodium chloride/200 mM HEPES/60 mM EDTA/1.0% Tween 20 pH=7.6) (Teknova) with 0.1% (w/v) bovine serum albumin. Each experiment began with two start up cycles with 60 s of contact time and a flow rate of 50 μL/min. For analysis methods, approximately 200-300 RUs of IgG antibodies was captured on each flow cell at a flow rate of 10 μL/min for 60 seconds. WT RBD or glycan variants samples were 5× serial diluted from 1000 nM in running buffer and flowed across the chip after capture at a 50 μL/min rate. The experiment had a 120 second contact time phase and 600 seconds dissociation phase. Regeneration was performed with 10 mM glycine at pH=1.5 at a flow rate of 50 μL/min for 30 seconds after each cycle. Kinetic fits were analyzed with 1:1 fitting and run through a script to filter out results that had poor fitting, low max RUs compared to expected, and kon and koff constants that fell outside of the range of measurement. Experiments that were flagged as poor-quality fitting by this script were not further analyzed.

### Pseudovirus Neutralization Assay

HEK293T (CRL-3216) and CHO cells (CRL-12023: double check) were obtained from ATCC (Manassas, VA, USA). Cells were maintained in DMEM supplemented with 10% fetal bovine serum (FBS) and 1% penicillin-streptomycin (P/S) antibiotic at 37° C. under 5% CO₂ atmosphere. For luciferase-based virus pseudoneutralization assays, HEK293T cells were transfected to produce SARS-COV-2 S containing pseudoviruses. Cells were seeded at 5 million cells onto T75 flasks and grown for 24 hours. Then, cells were treated with 48 μL GeneJammer (Agilent 204130-21), 6 μg S_IgE_deltaCterm19_plasmid (Genscript), and 6 μg pNL4-3.luc.R-E-backbone (Aldevron) and incubated for 48 hours. For variant pseudoviruses, cells were similarly treated with GeneJammer and backbone with 6 μg of S_SA_IgE_deltaCterm19, S_UK_IgE_deltaCterm19, or S_Brazil_IgE_deltaCterm19 plasmid. Transfection supernatants were then collected and supplemented with 12% FBS, sterile filtered, and stored at −80° C. Pseudovirus solutions were titered and dilution to working solutions set such that they yielded >215-fold greater relative luminescence units (RLS) than cells alone.

CHO cells expressing human ACE2 receptors (VCel-Wyb030) were obtained from Creative Biolabs (Shirley, NY). CHO-ACE2 cells were seeded at 10,000 cells/well in 96-well plates and incubated for 24 hours. Sera from vaccinated mice were heat inactivated at 56° C. for 15 minutes. 3-fold serial dilutions starting at 1:20 dilutions in DMEM supplemented with 10% FBS and 1% P/S were performed on sample sera and incubated for 90 minutes at room temperature with SARS-CoV2 pseudovirus based on concentrations determined from titering described above. Media containing diluted sera and pseudovirus were then applied to CHO-ACE2 cells. After 72 hours of incubation, cells were developed using BriteLite plus luminescence reporter system (Perkin Elmer 6066769) and signal measured using a plate reader (Biotek Synergy). Percent neutralization was calculated based on virus only positive control signal with background subtraction of cells only negative controls. ID₅₀ values were calculated using GraphPad Prism v8.0 nonlinear curve fitting with constraint Hill Slope <0.

### SARS-COV-2 Culture, Titer, and Neutralization Assay

SARS-Related Coronavirus 2, Isolate USA-WA1/2020, NR-52281 was deposited by the Centers for Disease Control and Prevention and obtained through BEI Resources, NIAID, NIH. All work with it was performed in the BSL-3 facility at the Wistar Institute. Vero cells (ATCC CCL-81) were maintained in antibiotic-free Dulbecco's modified Eagle's medium (DMEM) supplemented with 10% fetal bovine serum (FBS). To grow a stock of virus, 3 million Vero cells were seeded in a T-75 flask for overnight incubation (37□C, 5% CO2). The cells were inoculated the next day with 0.01 MOI virus in DMEM. Culture supernatant was harvested 3 days post infection, aliquoted, and stored at −80° C. For titering the virus stock, Vero cells were seeded in DMEM with 1% FBS at 20,000 cells/well in 96 well flat bottom plates for overnight incubation (37□C., 5% CO2). The USA-WA1/2020 virus stock was serially diluted in DMEM with 1% FBS and transferred in replicates of 8 to the previously seeded Vero cells. Five days post infection individual wells were scored positive or negative for the presence of cytopathic effect (CPE) by examination under a light microscope. The virus titer (TCID50/ml) was calculated using the Reed-Munch method and the Microsoft Excel based calculator published by Lei et al (Li et al., 2021, Virol Sin, 36(1): p. 141-144). For neutralization assays, Vero cells were seeded in DMEM with 1% FBS at 20,000 cells/well in 96 well flat bottom plates for overnight incubation (37 C, 5% CO₂). Serum samples were heat inactivated at 56° C. for 30 minutes. Serum samples were then serially diluted in DMEM with 1% FBS and 1% penicillin/streptomycin and incubated for one hour at room temperature with 300 TCID50/ml USA-WA1/2020. The serum-virus mixture was then transferred in triplicate to the previously seeded Vero cells. Five days post infection, individual wells were scored positive or negative for the presence of CPE and neutralization titers were calculated using the Reed-Munch method and a modified version of the Microsoft Excel based calculator published by Lei et al (Li et al., 2021, Virol Sin, 36(1): p. 141-144).

### Animal Studies

C57BL/6, BALBc, and K18-hACE2 mice were obtained from Charles River Laboratories (Malvern, PA) and The Jackson Laboratory (Bar Harbor, ME). Omni Mouse®; for human antibody studies were obtained from Ligand Pharmaceuticals Incorporated (San Diego, CA). All studies were performed in accordance with Wistar Institutional Animal Care and Use Committees under approved animal protocols. All animals were housed in the Wistar animal facility in ventilated cages and given free access to food and water. For the lethal challenge study, Texas Biomed were blinded to identity of vaccination groups and weight loss cutoff for euthanasia was 20%. For protein immunizations, mice were administered subcutaneously in 100 μL PBS co-formulated with RIBI adjuvant (Sigma Aldrich). For DNA immunizations, intramuscular injection with electroporation and sample collection. Plasmids were administered intramuscularly in 30 μL water into the tibialis anterior muscle. Electroporation was then performed using CELLECTRA EP delivery platform consisting of two pulse sets at 0.2 Amps at a 3 second interval. Each pulse set consists of two 52 ms pulses with 198 ms delay. At specified time points, blood was collected via submandibular vein puncture and centrifuged for 10 min at 15000 rpm to obtain sera. For cellular responses, mice were euthanized under CO₂ overdose. Spleens were collected into cold RPMI media supplemented with 10% FBS and 1% P/S.

Female Hartley guinea pigs (8 weeks old, Elm Hill Labs, Chelmsford MA) were housed at Acculab (San Diego CA). On day 0 and day 28 animals were anaesthetized with isoflurane vapor and received intradermal Mantoux injections of 100 μL 10, 5 or 0.5 μg pDNA immediately followed by CELLECTRA-3P electroporation. The CELLECTRA® EP delivery consists of two sets of pulses with 0.2 Amp constant current. Second pulse set is delayed 3 seconds. Within each set there are two 52 ms pulses with a 198 ms delay between the pulses. Serum samples were collected by jugular or saphenous blood collection throughout the study on days 0, 7, 14, 21, 28 and 42. Whole blood samples to process PBMCs for cellular assay were collected from the jugular vein on days 14 and 42. All animals were housed in the animal facility at Acculab Life Sciences (San Diego, CA). All animal protocols were approved by Acculab Institutional Animal Care and Use Committees (IACUC).

Golden Syrian hamsters (8 weeks old, Envigo, Indianapolis, IN) were housed at Acculab (San Diego, CA). Hamsters received intramuscular (IM) injections of 60 μL of 2 or 10 μg pDNA formulation into the tibialis anterior muscle immediately followed by electroporation with the CELLECTRA-3P device under Isoflurane vapor anesthesia at day 0 and day 21. The CELLECTRA® EP delivery consists of two sets of pulses with 0.2 Amp constant current. Second pulse set is delayed 4 s. Within each set there are two 52 ms pulses with a 198 ms delay between the pulses. Serum samples were collected at indicated timepoints via saphenous vein blood collection throughout the experiment. All animals were housed in the animal facility at Acculab Life Sciences (San Diego, CA). All animal protocols were approved by Acculab Institutional Animal Care and Use Committees.

In-vivo study was concluded with terminal blood, lung lavage and nasal wash collection. Lavage buffer was prepared as PBS containing 100 uM EDTA, 0.05% Sodium Azide, 0.05% Tween-20 and Protease Inhibitor. Hamsters were euthanized by jugular exsanguination with intraperitoneal (IP) injection of 86.7 mg/kg pentobarbital sodium or overdose Isoflurane gas inhalation. Euthanized hamster was placed in supine position and skin was disinfected using 70% Isopropyl alcohol. A longitudinal cut using scissors and blunt dissection along the midline of the neck was performed to expose the trachea. An opening into the exposed trachea was created by making a transverse, semilunar cut using #11 blade.

To collect nasal wash an 18ga blunt end needle was inserted toward the nose and gently proceeded upwards until reaching the nasal palate. A syringe filled with 1.5 mL lavage buffer was connected to the blunt end needle and correct placement was tested by dispensing a small amount through the hamster's nares. The entire volume of lavage fluid was rapidly dispensed and collected directly from the nares into a 5.0 mL Eppendorf tube.

To collect bronchioalveolar lavage (BAL), an 18ga blunt end needle, attached to a three-way stopcock and primed with lavage buffer (approximately 0.5 mL) to eliminate empty airspace, was inserted forward until just prior to the tracheal bifurcation into the lungs. The blunt end needle was secured in the trachea with a silk 2-0 tie. A 3 mL receiver syringe and a 10 ml syringe filled with 9 mL of lavage buffer was connected to the blunt end needle via the three-way stopcock. The lungs were rinsed three times (3 mL each time) with a total of 9 mL lavage buffer. Typically, 50% of lavage buffer was recovered.

### Hamster Biodistribution

Lung lavage and nasal wash samples were ultrafiltrated using a 2 mL 100 kDa cut-off ultrafiltration device (Millipore, Burlington MA) spinning 1 mL BAL or NW for 15 min at 4000 g. Ultrafiltrated BAL was diluted 1:6 and nasal wash was diluted 1:4 in ELISA dilution buffer and following washes and blocking as described in the ELISA section added to half area assay plates (Costar) coated with 25 μL/well of 1 μg/mL SARS-COV-2 RBD (Sinobiological) in dilution buffer overnight at 4 C. BAL and NW samples were tested at a 7-step 1:2 serial dilution.

### Negative-Stain Electron Microscopy

Purified RBD g5.1 nanoparticle was dialyzed into 20 mM HEPES buffer, 0.15M NaCl, pH 7.4. A total of 3 μL of purified proteins was adsorbed onto glow discharged carbon-coated Cu400 EM grids. The grids were then stained with 3 μL of 2% uranyl acetate, blotted, and stained again with 3 μL of the stain followed by a final blot. Image collection and data processing was performed on a FEI Tecnai T12 microscope equipped with Oneview Gatan camera at 90 450× magnification at the camera and a pixel size of 1.66 Å.

### Cryo Electron Microscopy

Cryo-EM vitrification was obtained in a Vitrobot Mark IV robot (FEI). Four μL of purified RBD g5.1 24mer nanoparticles in 1×PBS were deposited on a glow-discharged holey carbon grid (C-flat 1.2/1.3, 300 mesh; Protochips). Excess liquid was blotted away followed by immediate plunging into liquid ethane cooled by liquid nitrogen. The vitrified specimen was then introduced into an FEI Talos Arctica electron microscope (FEI). Automated data collection was performed in EPU (FEI) and 640 movie micrographs were recorded with a Falcon 3 camera (FEI) at 150,000× magnification corresponding to an image pixel size of 0.97 Å on the object scale. Each movie micrograph comprised 50 frames, each frame was exposed with a dose of ˜1 e⁻/Å². Data processing was performed in Relion v3.1.2 (Scheres, 2012, J Mol Biol, 2012. 415(2): p. 406-18). Movie micrograph frame alignment, spectral signal weighing and summation was followed by CTF modeling (CTFFIND4 (Rohou et al., 2015, J Struct Biol, 192(2): p. 216-21). Candidate molecular projection images were identified with Relion LoG picking (˜271,000). Image windows corresponding to the candidate molecular projection image coordinates were extracted and binned by a factor of 2. The extracted binned data was subjected to 2D classification. Manual inspection of class averages led to identification of 93,348 molecular projection images selected for further data processing. Molecular projections were re-extracted unbinned from the summed micrographs and iterative Euler angular reconstitution and 3D object reconstruction was performed with a low-resolution ferritin density map as initial seed. 3D refinement was performed both asymmetrically (FSC 0.143 resolution 3.98 Å) and under the assumption of octahedral symmetry (FSC 0.143 resolution 3.42 Å). Since the objective was to map the attachment sites of the RBDs to the ferritin cage, no efforts were made to improve ferritin particle alignment in the refinement strategy for the present manuscript.

### ELISpot Assay

Spleens from immunized mice were processed by a tissue stomacher, and red blood cells were then lysed by ACK buffer (Thermo Fisher Scientific). Single cell suspension was counted, and 2×10⁵ splenocytes were plated into each well of the Mouse IFN-γ ELISpotPLUS plates (MabTech). The splenocytes were stimulated for 20 hours at 37° C. with RBD peptides (15-mer peptides overlapping by 9 amino acid spanning the RBD of SARS-COV-2 spike protein, GenScript), at 5 μg/mL of each peptide in RPMI+10% FBS (R10). The spots were developed according to manufacturer's instructions. R10 and cell stimulation cocktails (Invitrogen) were used for negative and positive controls, respectively. Spots were scanned and quantified by ImmunoSpot CTL reader. Spot-forming unit (SFU) per million cells was calculated by subtracting the negative control wells.

### Intracellular Cytokine Staining and Flow Cytometry

Splenocytes were processed as described in the previous section and stimulated with RBD peptides for 5 hours at 37° C. with protein transport inhibitor (Invitrogen) and anti-mouse CD107a-FITC antibody (BioLegend). Cell stimulation cocktail and R10, with protein transport inhibitor, were used as positive and negative controls, respectively. After stimulation, cells were stained with Live/Dead violet (Invitrogen) for viability. Anti-mouse CD4-BV510, CD8-APC-Cy7, CD44-A700, and CD62L-BV711 antibodies were used for surface staining and CD3e-PE-Cy5, IFN-γ-APC, and TNF-α-BV605 (all from BioLegend) were used for intracellular staining. The samples were run on an 18-color LSRII flow cytometer (BD Biosciences) and analyzed by FlowJo software.

### Competition Assay

96-well Flat-Bottom Half-Area plates (Corning) were coated at room temperature for 8 hours with 1 μg/mL 6×-His tag polyclonal antibody (PA1-983B, ThermoFisher), followed by overnight blocking with blocking buffer containing 5% milk/1×PBS/0.01% Tween-20 at 4° C. The plates were then incubated with RBD at 1 μg/mL at room temperature for 1-2 hours. Mouse Sera (BALB/c, terminal bleeds, week 6, n=5) either immunized with RBD-WT or RBD-gPenta was serially diluted 3-fold starting at 1:20 with dilution buffer (5% milk/1×PBS/0.01% Tween-20) was added to the plate and incubated at room temperature for 1-2 hours. Plates were then washed and incubated at room temperature for 1 hour with ACE2-IgHu at a constant concentration of 0.06 μg/mL diluted with the dilution buffer. After being washed, the plates were further incubated at room temperature for 1 hour with goat-anti human IgG-Fc fragment cross-adsorbed Ab (A80-340P; Bethyl Laboratories) at a 1:10,000 dilution, followed by addition of TMB substrates (ThermoFisher), and then quenched with 1M H₂SO₄. Absorbances at 450 nm and 570 nm were recorded with a BioTek plate reader. Four washes were performed between every incubation step using PBS and 0.05% Tween-20. The assay was performed in triplicates. The average absorbance of the lowest dilutions with saturating ACE2 signals was calculated to get a maximum ACE2 binding and no blocking. Each average absorbance value was subtracted from the maximum to get an ACE2 blocking curve. The blocking titer is defined as the reciprocal of the highest dilution where two consecutive dilutions have readings below zero. The maximum area under the curve is determined by calculating the Area Under the Curve (AUC) of full ACE2 binding without the competitor. The AUC of the competitor is then subtracted from the maximum AUC which provides the area between the two curves (blocking area) and is a measure of ACE2 blocking. The fraction ACE2 blocking is defined as the fraction of the blocking area to the maximum AUC.

### Site-Specific Glycan Analysis Using Mass Spectrometry

RBD proteins were denatured for 1 h in 50 mM Tris/HCl, pH 8.0 containing 6M of urea and 5 mM dithiothreitol (DTT). Next, the proteins were reduced and alkylated by adding 20 mM iodacetamide (IAA) and incubated for 1 h in the dark, followed by a 1 h incubation with 20 mM DTT to eliminate residual IAA. The alkylated proteins were buffer-exchanged into 50 mM Tris/HCl, pH 8.0 using Vivaspin columns (3 kDa) and digested separately overnight using three protease enzymes in separate tubes, trypsin, (Mass Spectrometry Grade, Promega), Chymotrypsin (Mass Spectrometry Grade, Promega), Alpha-lytic protease (Sigma-Aldrich) at a ratio of 1:30 (w/w). The next day, the peptides were dried and extracted using C18 Zip-tip (MerckMilipore). The peptides were dried again, re-suspended in 0.1% formic acid and analyzed by nano LC-ESI MS with an Ultimate 3000 HPLC (Thermo Fisher Scientific) system coupled to an Orbitrap Eclipse mass spectrometer (Thermo Fisher Scientific) using stepped higher energy collision-induced dissociation (HCD) fragmentation. Peptides were separated using an Easy Spray PepMap RSLC C18 column (75 μm×75 cm). A trapping column (PepMap 100 C18 column 3 μM 75 μm×2 cm) was used inline prior to separation with the analytical column. The LC conditions were as follows: 280-minute linear gradient consisting of 4-32% acetonitrile in 0.1% formic acid over 260 minutes followed by 20 minutes of alternating 76% acetonitrile in 0.1% formic acid and 4% acetonitrile in 0.1% formic acid, used to ensure all the samples has eluted from the column. The flow rate was set to 200 nL/min. The spray voltage was set to 2.7 kV and the temperature of the heated capillary was set to 40° C. The ion transfer tube temperature was set to 275° C. The scan range was 375-1500 m/z. The stepped HCD collision energy was set to 15%, 25%, 45%, appropriate for fragmentation of glycopeptide ions. Precursor and fragment detection were performed using an Orbitrap at a resolution MS1=100,000. MS2=30,000. The AGC target for MS1 was set to standard and injection time set to auto which involves the system setting the two parameters to maximize sensitivity while maintaining cycle time. Full LC and MS methodology can be extracted from the appropriate raw file using XCalibur FreeStyle software.

Glycopeptide fragmentation data were extracted from the raw file using Byonic™ (Version 4.0; Protein Metrics Inc.) and Byologic™ software (Version 4.0; Protein Metrics Inc.). The glycopeptide fragmentation data were evaluated manually for each glycopeptide; the peptide was scored as true-positive when the correct b and y fragment ions were observed along with oxonium ions corresponding to the glycan identified. The MS data was searched using the Protein Metrics 305 N-glycan library with sulfated glycans added manually. The relative amounts of each glycan at each site as well as the unoccupied proportion were determined by comparing the extracted chromatographic areas for different glycotypes with an identical peptide sequence. The precursor mass tolerance was set at 4 ppm and 10 ppm for fragments. A 1% false discovery rate (FDR) was applied. The relative amounts of each glycan at each site as well as the unoccupied proportion were determined by comparing the extracted ion chromatographic areas for different glycopeptides with an identical peptide sequence.

Glycans were categorized according to the composition detected. HexNAc(2)Hex(10+) was defined as M9Glc, HexNAc(2)Hex(9-5) was classified as M9 to M3. Any of these structures containing a fucose were categorized as FM (fucosylated mannose). HexNAc(3)Hex(5-6)X was classified as Hybrid with HexNAc(3)Fuc(1)X classified as Fhybrid. Complex-type glycans were classified according to the number of processed antenna and fucosylation, HexNAc(3)Hex(3-4)X, HexNAc(4)X, HexNAc(5)X, and HexNAc(6)X is assigned. As this fragmentation method does not provide linkage information compositional isomers are group, so for example a triantennary glycan contains HexNAc 5 but so does a biantennary glycans with a bisect. Core glycans refer to truncated structures smaller than M3. M9glc-M4 were classified as oligomannose-type glycans. Glycans containing at least one sialic acid or one sulfate group were categorized as NeuAc and sulfated respectively. Further, with in complex-type and hybrid-type glycans, glycan compositions have been categorized as, agalactosylated, galactosylated and sialylated. HexNAc(3)Hex(3-4)X, HexNAc(4)XHex(≤3), HexNAc(5)XHex(≤3), and HexNAc(6)XHex(≤3) is assigned as Agalactosylated. HexNAc(3)Hex(3-4)X, HexNAc(4)XHex (>3), HexNAc(5)XHex (>3), and HexNAc(6)XHex (>3) is assigned as galactosylated. The complex-type and hybrid type glycans with NeuAc are assigned as sialylated.

### Results

### Mapping Antigenic Effects of N-Linked Glycans on the RBD

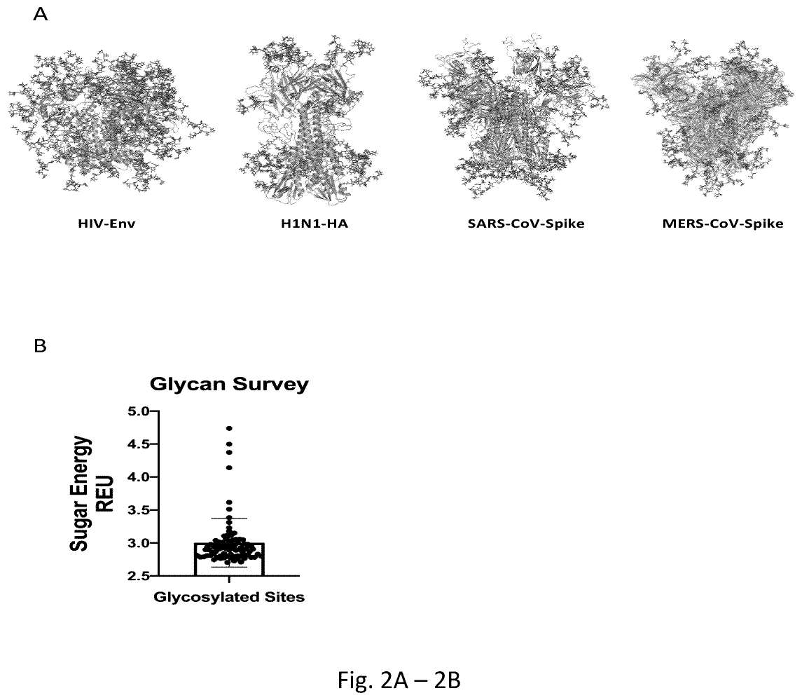

To assess the feasibility of adding N-linked glycans to alter antibody responses to RBD (FIG. 1A), an advanced structural algorithm was built called Cloaking With Glycans (CWG) for modeling every possible glycan on the RBD (FIG. 1B). The PNGS positions were filtered if the asparagine had low solvent accessibility or high clash score (FIG. 1C). Next, energetics of naturally occurring glycans were surveyed (FIGS. 2A, 2B) and glycan energy filters were employed for the designed glycan positions, as well as filters for protein folding energies and structural considerations. This process led to the identification of 43 out of 196 positions for experimental characterization (FIGS. 1C, 1D).

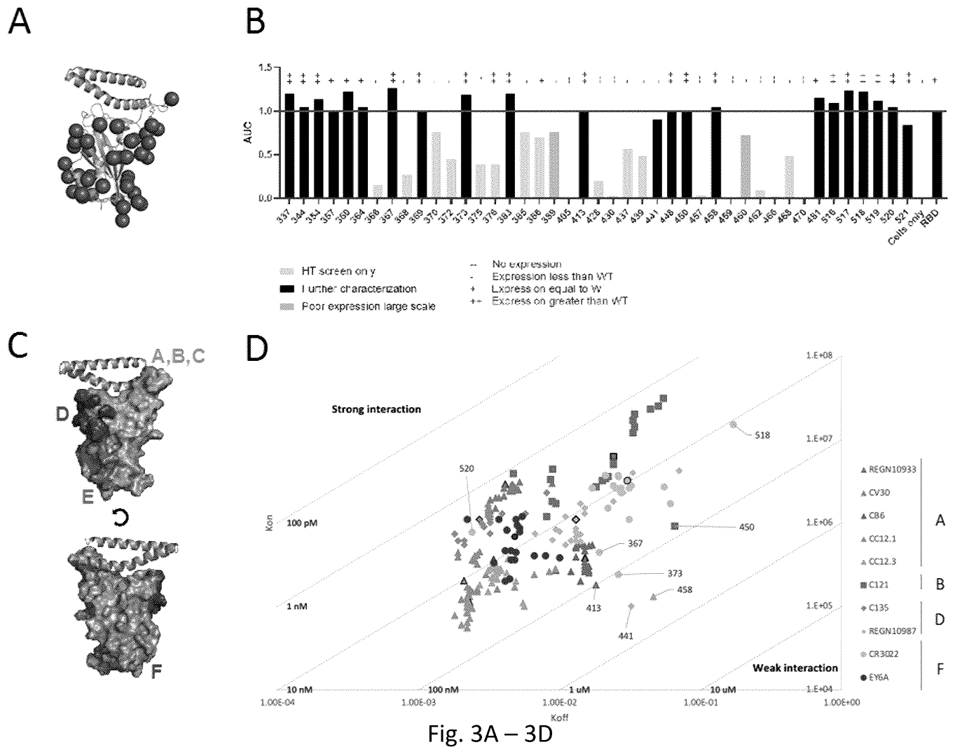

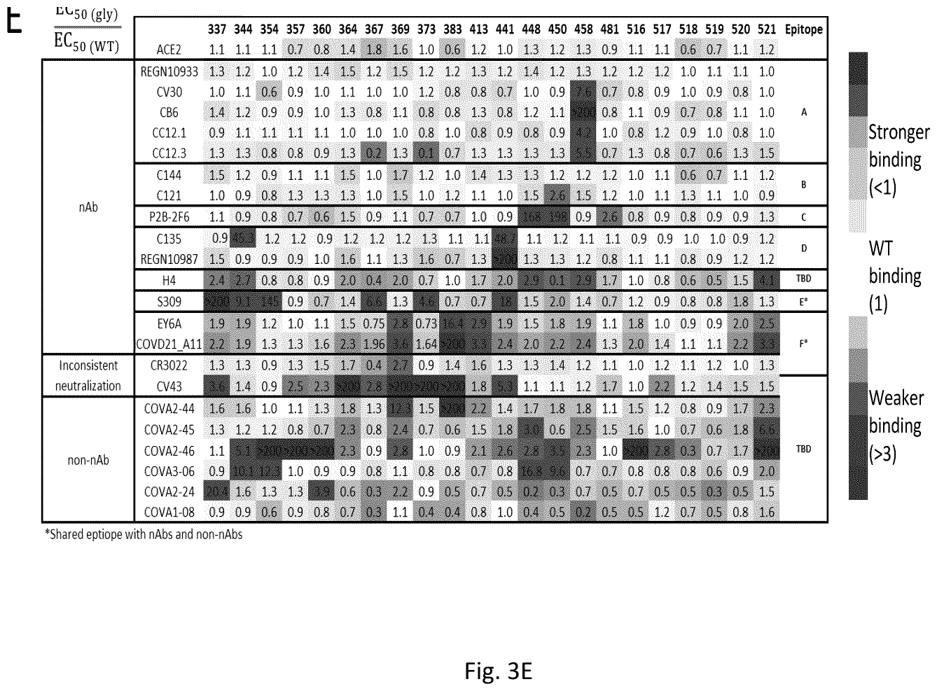

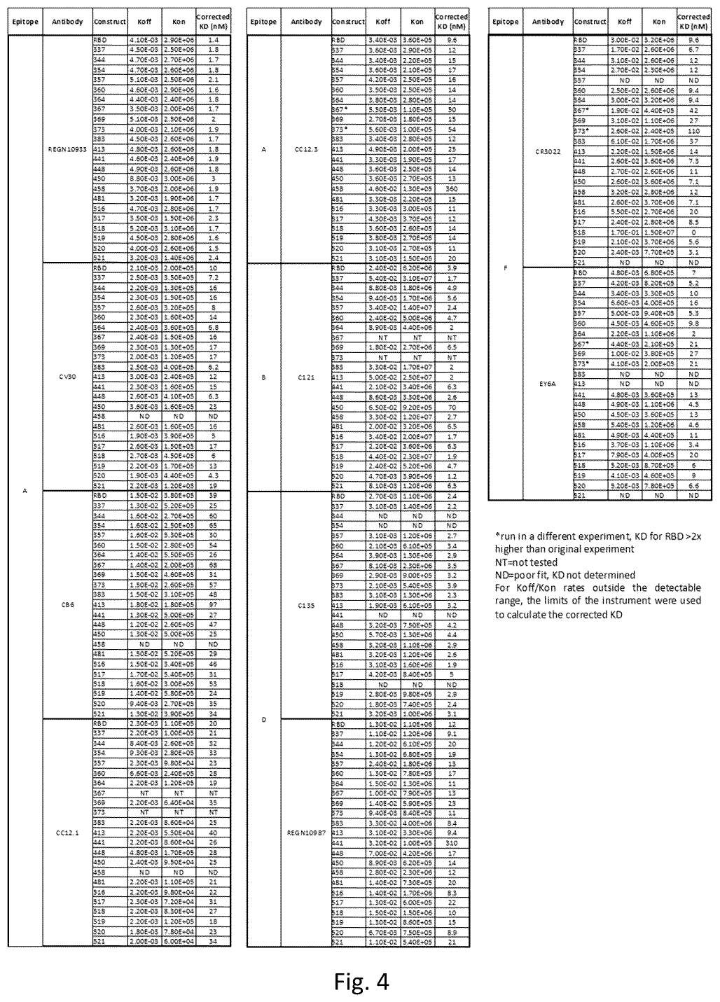

To assess the impact on RBDs harboring single glycan mutants (FIG. 3A) in vitro, each variant was produced and biophysical and antigenic profiles were measured. The glycan variants were synthesized and screened for expression and binding to ACE2 in a high-throughput, small-scale transfection format and downselected to 22 variants for further evaluation (FIG. 3B). To characterize the antigenic properties of the glycan variants, 14 RBD-directed nAbs, 2 Abs with inconsistent neutralization (Huo et al., 2020, Cell Host Microbe, 28(3): p. 497; Yuan et al., 2020, Science, 368(6491): p. 630-633; Zhou et al., 2020, Nat Struct Mol Biol, 27(10): p. 950-958; Seydoux et al., 2020, Immunity, 53(1): p. 98-105 e5), and 6 non-nAbs (Yuan et al., 2020, Biochem Biophys Res Commun, 538:192-203; Brouwer et al., 2020, Science, 369(6504): p. 643-650; Ju et al., 2020, Nature, 584(7819): p. 115-119; Rogers et al., 2020, Science, 369(6506): p. 956-963, Zhou et al., 2020, Nat Struct Mol Biol, 27(10): p. 950-958; Seydoux et al., 2020, Immunity, 53(1): p. 98-105 e5; Hansen et al., 2020, Science, 369(6506): p. 1010-1014; Wu et al., 2020, Science, 368(6496): p. 1274-1278; Shi et al., 2020, Nature, 584(7819): p. 120-124; Pinto et al., 2020, Nature, 583(7815): p. 290-295; Robbiani et al., 2020, Nature, 584(7821): p. 437-442, Tian et al., 2020, Emerg Microbes Infect, 9(1): p. 382-385) were utilized. Most nAbs target epitopes in the RBS (RBD-A, RBD-B, RBD-C) (Yuan et al., 2020, Biochem Biophys Res Commun, 538:192-203) and some target outside the RBS (Barnes et al., 2020, Nature, 588(7839): p. 682-687) (RBD-D, RBD-E and RBD-F) (FIG. 3C). In general, it was sought to identify glycans that do not interfere with nAb binding and block non-nAbs. The reactivity of the set of antibodies to each glycan mutant was determined by SPR and ELISA (FIG. 3D,3E, FIG. 4). Reduced binding of neutralizing RBD-A, RBD-B. RBD-C or RBD-D antibodies was observed in the presence of glycans at residues 441 (RBD-D), 448, 450 and 481 (RBD-B,-C), and 458(RBD-A). Glycans at these five positions do not significantly impact ACE2 binding, suggesting SARS-COV-2 variant viruses harboring spike proteins with these glycans could evade immune pressure against those epitopes. In addition, glycans at positions 337, 344, 354, 357, 360, 369, 383, 448, 450, 516 and 521 show dramatically reduced binding to non-nAb(s). Effects on binding to the antibody panel were not observed for glycans at 518, 519 and 520. Similar antigenic patterns were noticed in glycan positions that reduce binding to some of the non-nAbs as well as nAbs in RBS-E and RBS-F, suggesting there is overlap in these nAb and non-nAb epitopes. In sum, the experimental screening exhaustively evaluated the effect of N-linked glycans on the expression and antigenic profile of the RBD.

### N-Linked Glycan Decoration Improves RBD Directed Immunity

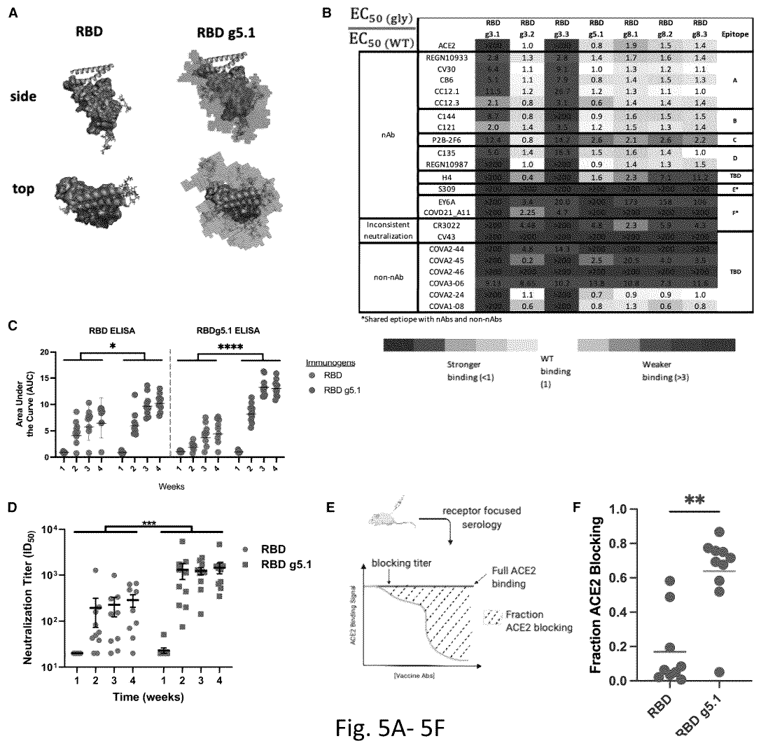

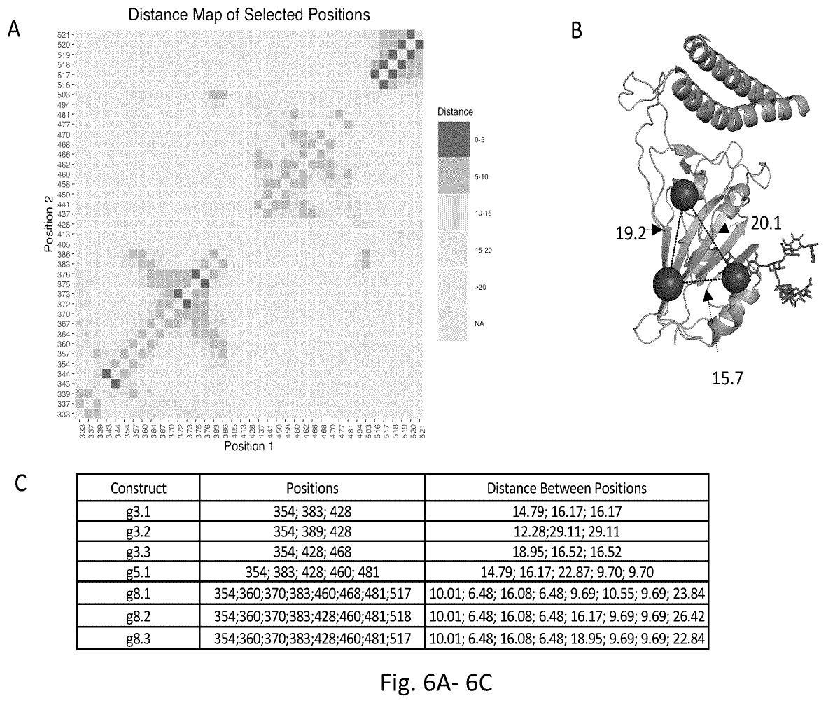

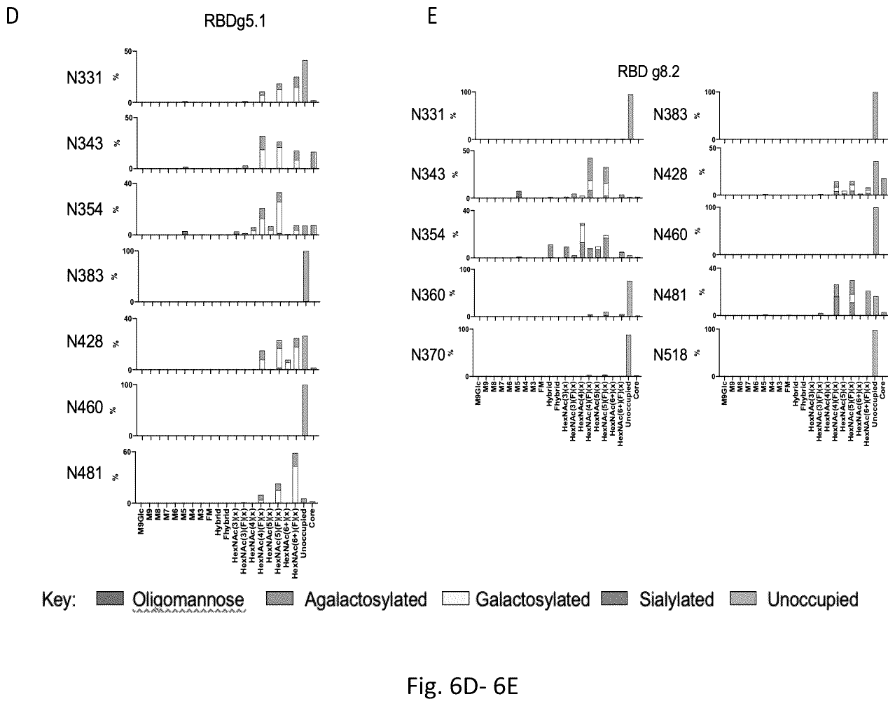

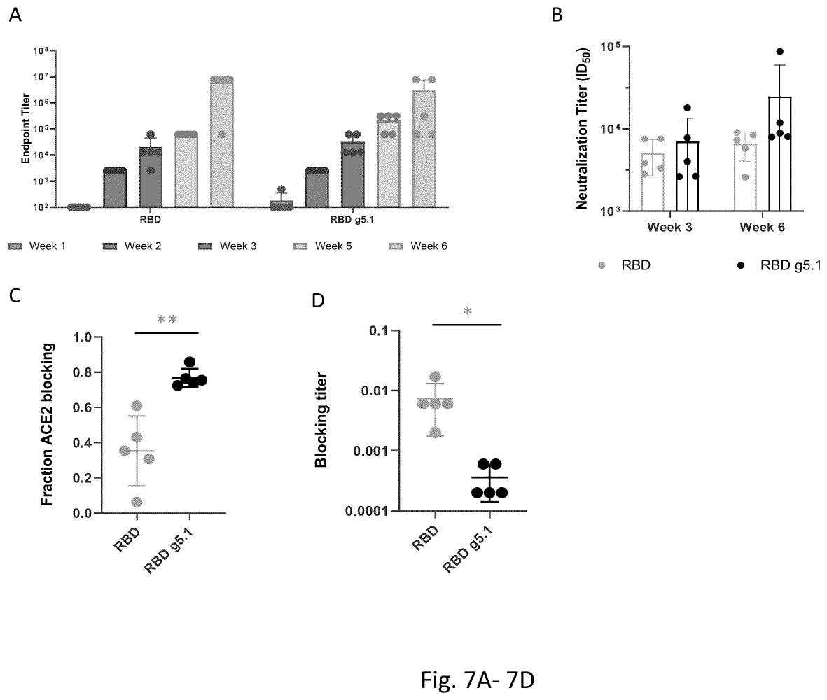

The single glycan data was utilized to add sets of glycans to the RBD that maximally cover multiple non-neutralizing epitopes and preserve accessibility to RBS targeted neutralizing epitopes. To this end, a glycan distance map was constructed allowing design of three, five and eight glycan combinations which were experimental tested to determine if the sets could provide optimized antigenic profiles (FIG. 5A, 6A, 6B, 6C). Two of the three glycan variants (g3.1 and g3.3) had heavily reduced binding to all antibodies in the panel. Eight glycan variants (g8.1, g8.2 and g8.3) had slightly reduced EC₅₀ to nearly all the RBD neutralizing antibodies (FIG. 5B). However, both g3.2 and g5.1 (harboring three and five glycans, respectively) bound well to nAbs and had reduced affinity for non-nAbs (FIG. 5B). Since new non-nAbs may be identified in the future, the remaining experiments focused on the more glycosylated variants (i.e. g5.1 over g3.2), since they are more likely to reduce accessibility to epitopes recognized by non-nAbs. To determine the glycan composition of g5.1, a single-site glycan analysis was performed by LC-MS (Watanabe et al., 2020, Science, 369(6501): p. 330-333). To convey the occupancy and processing states at each PNGS, the abundances of each glycan were determined as: Oligomannose-type (high mannose, M3GlcNAc2 to M9GlcNAc2, including fucosylated mannose), hybrid- and complex-type divided in three subgroups; agalactosylated (contains no galactose), galactosylated (containing at least one galactose), and sialylated (containing at least one sialic acid), and unoccupied (no glycan). Core glycans were also included in the analysis, which represents truncated glycan groups i.e., compositions smaller than HexNAc2Hex3. The N331, N343, N354, N428 and N481 show occupancy of galactosylated complex-type glycans, while N383 and N460 are unoccupied (FIG. 6D). Interestingly, g5.1 has more occupied glycans than g8.2, suggesting g5. 1 may be superior for focusing antibody responses (FIG. 6D, 6E). To assess immune focusing of g5.1, BALB/c mice were immunized with a single 10 μg injection of DNA plasmids encoding wild-type(WT) or g5.1 RBD (all immunizations in the manuscript are with DNA plasmids unless otherwise noted). In the immune focused group (RBDg5.1), higher titers of antibodies were observed as assessed by Area-Under-the-Curve (AUC) analysis against both RBD and RBDg5.1 antigens (FIG. 5C). The RBDg5.1 immunized animals also produced higher titers of neutralizing antibodies (FIG. 5D). To investigate the difference in specificity of the RBD elicited responses, an ACE2 blocking assay (Walker et al., 2020, J Clin Microbiol, 58(11)) was employed (FIG. 5E). It was observed that RBD g5.1 elicited significantly more ACE2-blocking antibodies than WT RBD, suggesting g5.1 is immune focusing antibodies to the RBS (FIG. 5E, 5F). In a prime-boost experiment using a much larger 25 μg dose, similar immunogenicity was observed between RBD groups but higher ACE2-blocking from animals immunized with the immune-focused RBDg5.1 (FIG. 7). Together this data demonstrates that combinations of strategically selected glycans reduce the affinity of non-nAbs and can focus immune responses to the neutralization-rich RBS or other epitopes of interest.

### DNA-Launched Nanovaccines Amplify and Accelerate Immune Responses

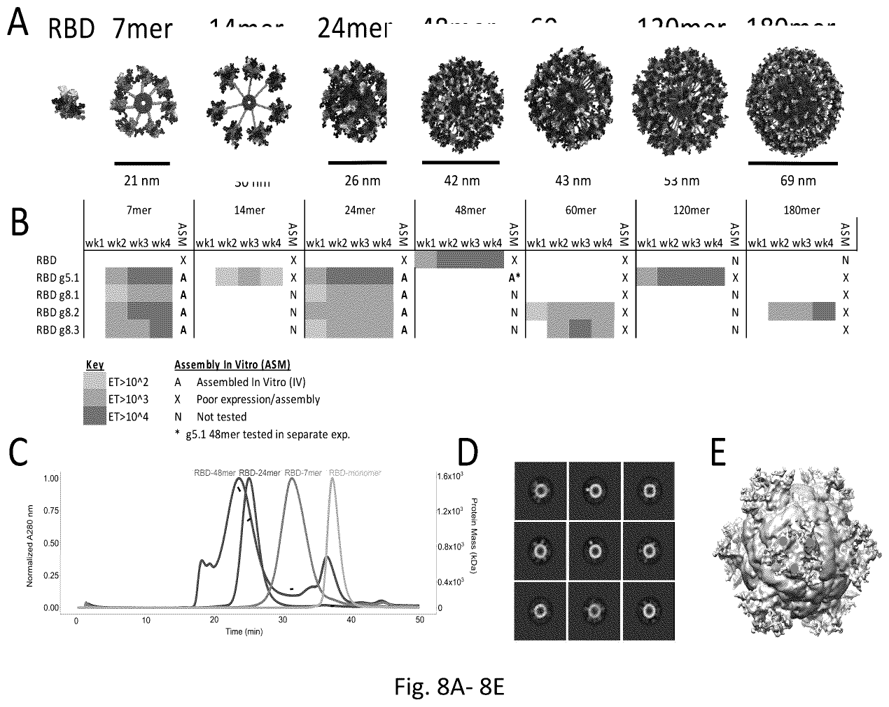

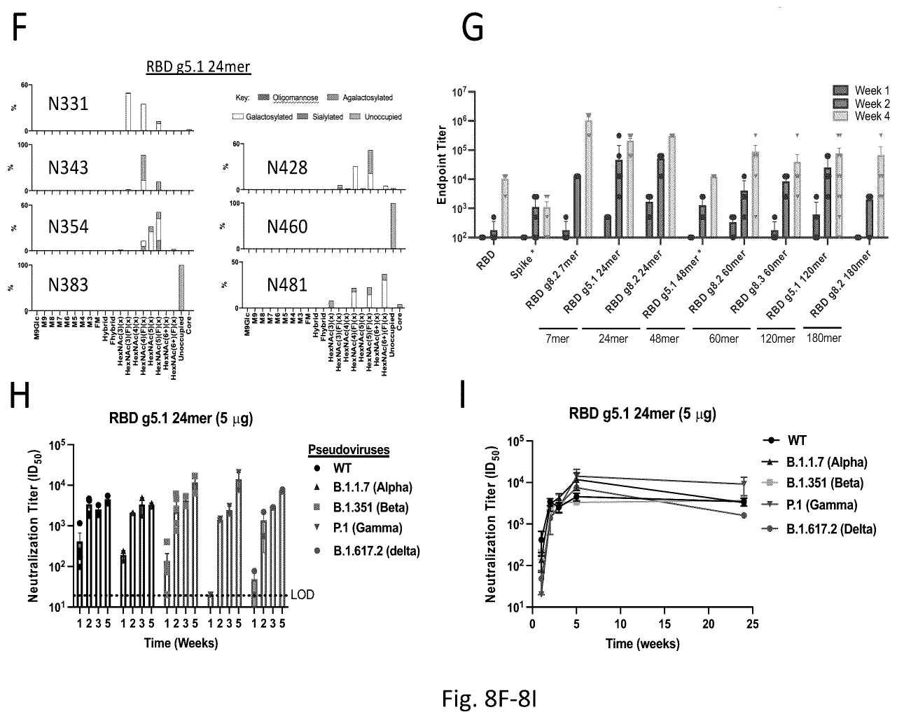

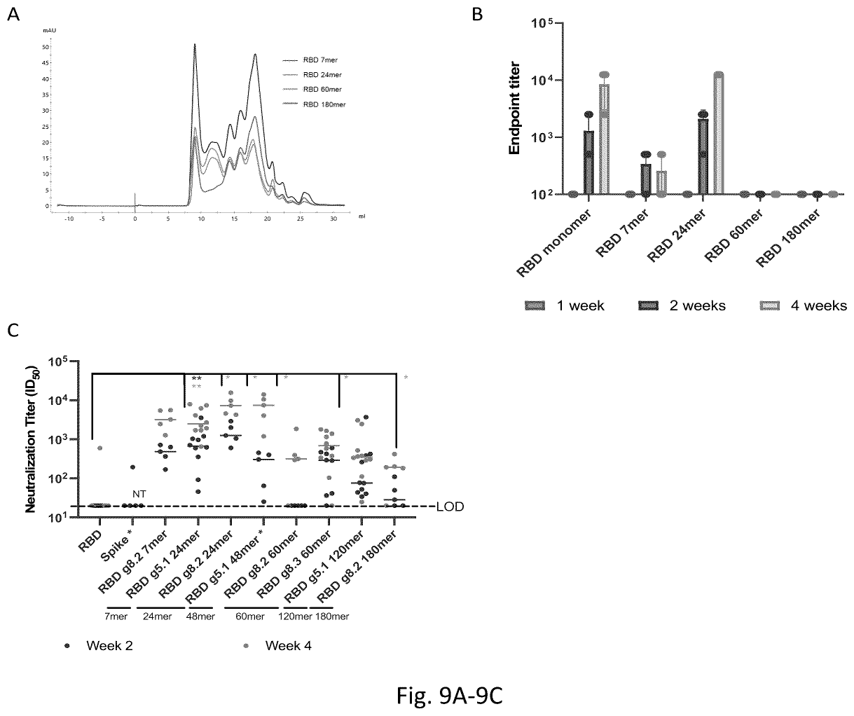

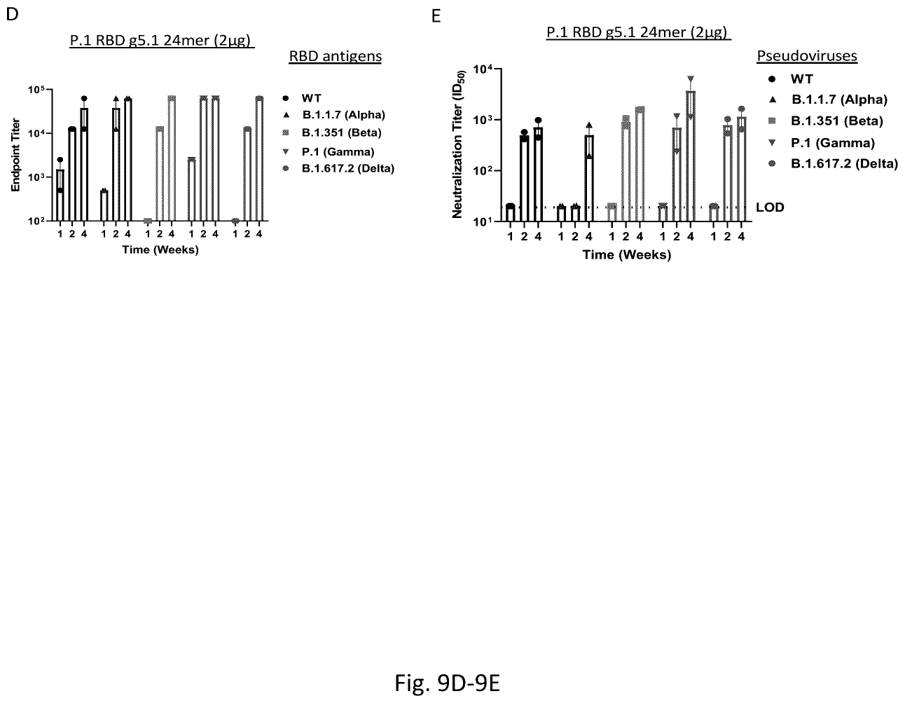

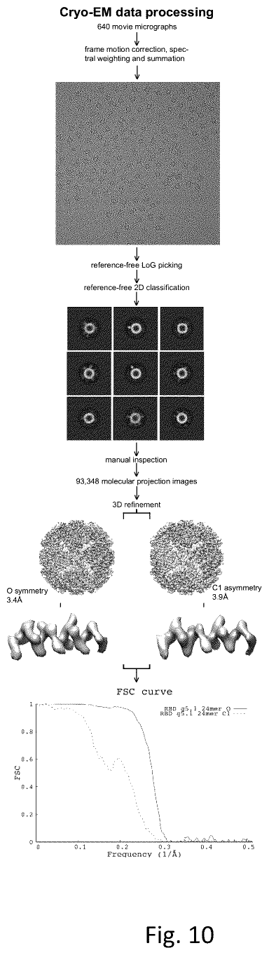

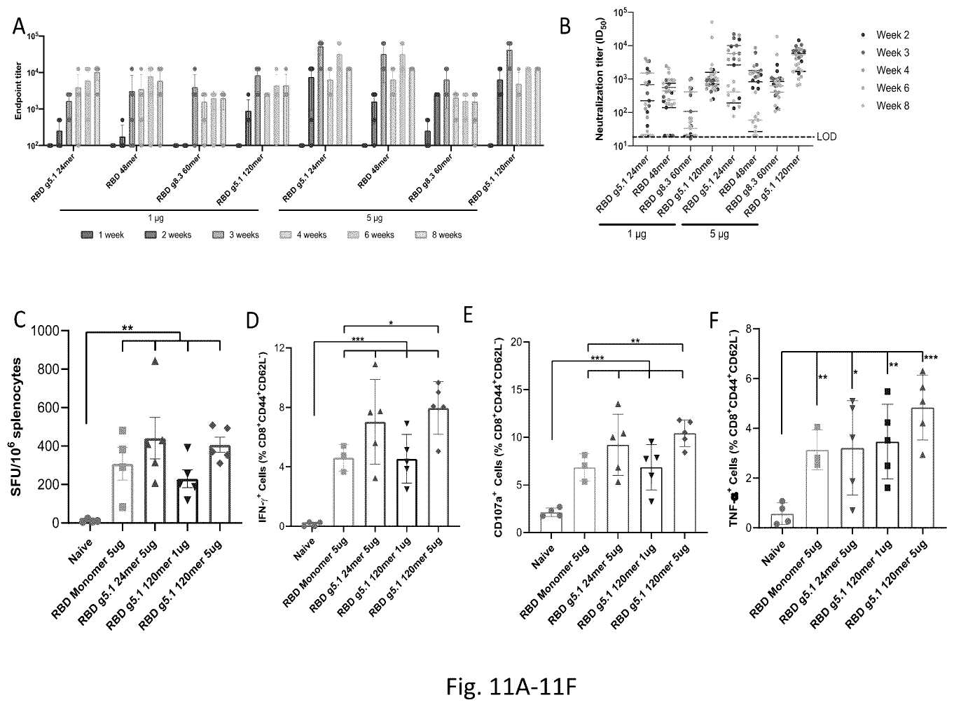

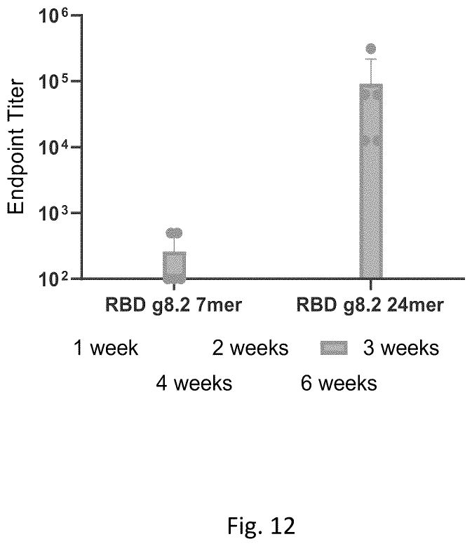

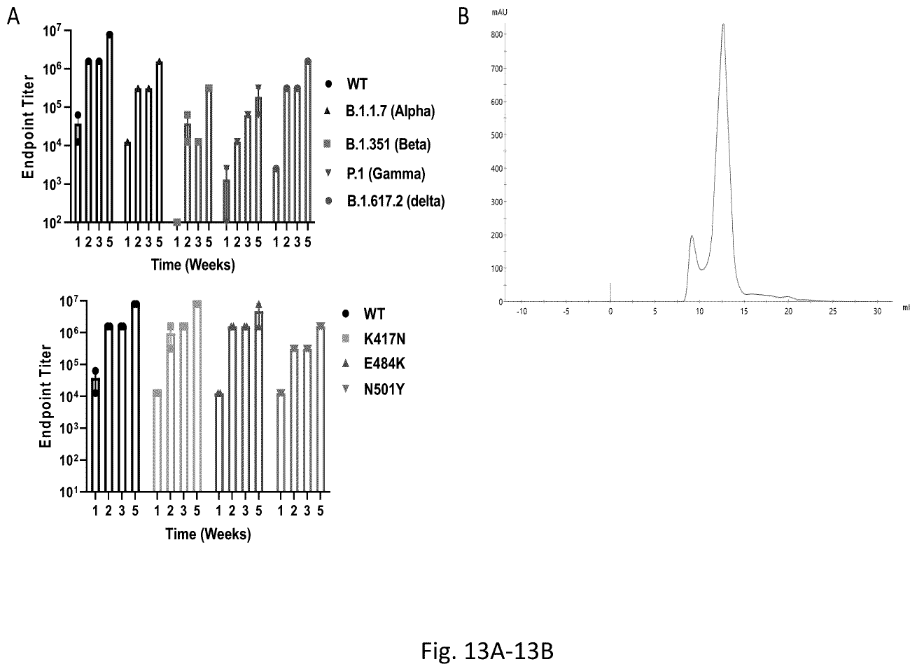

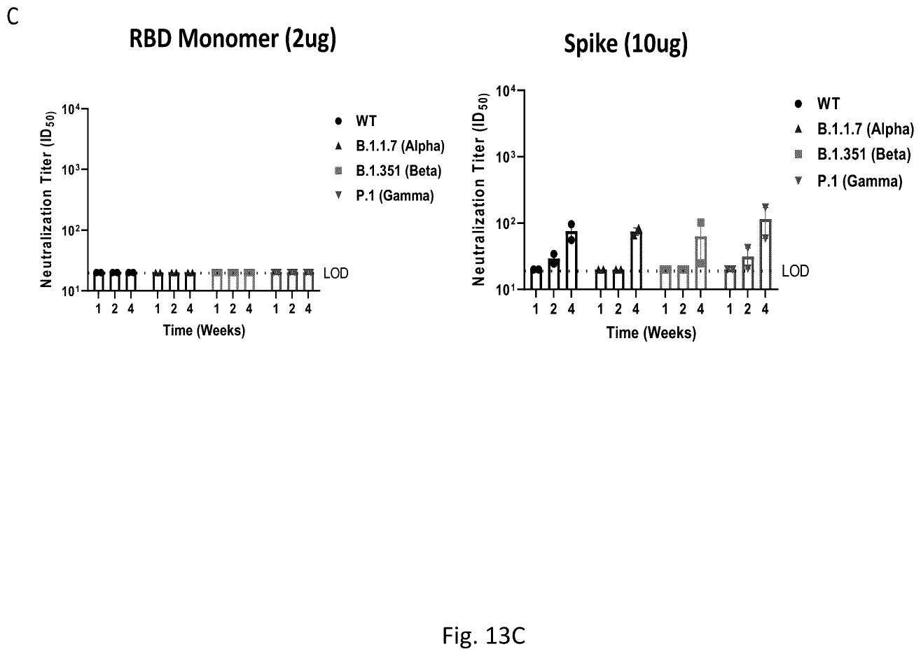

To develop multivalent vaccines, RBDs were genetically fused to a set of four different self-assembling scaffold proteins (Xu et al., 2020, Adv Sci (Weinh), 7(8): p. 1902802; Manolova et al., 2008, Eur J Immunol, 38(5): p. 1404-13; Kelly et al., 2019, Expert Rev Vaccines, 18(3): p. 269-280; Zhao et al., 2014, Vaccine, 32(3): p. 327-37) with a potent CD4-helper epitope(LS-3) to help enhance germinal center responses (Xu et al., 2020, iScience, 23(8): p. 101399). Tandem repeats of RBD have been shown to improve neutralization titers by 10-100 fold (Dai et al., 2020, Cell, 182(3): p. 722-733 e11), thus dimers of RBDs were displayed on some of the self-assembling scaffolds as well. Nanoparticles were engineered using the computational design methods (Xu et al., 2020, Adv Sci (Weinh), 7(8): p. 1902802), resulting in display of 7, 14, 24, 48, 60, 120 or 180 RBDs (FIG. 8A). 19 nanoparticles were rapidly screened directly in vivo using a single mouse per construct at a single low dose of 2 μg. 14 of the 19 nanoparticles were immunogenic (FIG. 8B). Strikingly, rapid antibody responses were detected just 1 week after immunization with RBD g5.1 24mer, RBD 48mer and RBD g5.1 120mer (FIG. 8B). In parallel, nanoparticles were expressed and purified in vitro. In contrast to wild type RBD multimers, which could not be purified and were not more immunogenic than RBD monomer (FIG. 9A, 9B), nine glycan modified RBD multimers were purified as assessed by size exclusion chromatography with multiangle light scattering (FIG. 8C). To further confirm assembly of the RBD g5.1 24mer, structural analysis by cryo electron microscopy (cryo-EM) was employed for RBD g5.1 24mer (FIG. 8D, 8E, FIG. 10). Single glycan analysis of RBD g5.1 24mer shows similar occupancy to RBD g5.1 monomer, however full occupancy was observed with highly processed-type glycans at N331 and N428 on the RBD g5.1 nanoparticle (FIG. 8F). Immunizations were observed with selected constructs in BALB/c mice (n=5 or n=10) using a single low dose of 2 μg (FIG. 8G). At this single low dose, all the glycan-modified nanoparticle groups were more immunogenic than RBD or full-length Spike. RBD g5.1 24mer and 120mer both generated strong binding and neutralizing responses (week 4 mean ID₅₀ of 3677 and 791, respectively) (FIG. 8G and FIG. 9C). In C57BL/6 mice, similar immunogenicity was observed at lug and 5 μg doses for select nanoparticles including improvements in CD8+ T cells (FIG. 11A-11E). RBD g8.2 7mer and RBD g8.2 24mer elicit similarly strong humoral responses when administered as purified protein nanoparticles (FIG. 12) Strikingly, strong binding and neutralizing responses were observed in BALB/c mice immunized with 5 μg RBD g5.1 24mer plasmid against the emergent UK (B.1.17, Alpha), South Africa (B.1.351, Beta), Brazilian (P.1, Gamma) and Indian (B.1.617.2, Delta) variants, indicating cross-reactivity and strong potential relevance against emerging variants (FIG. 8H, 13A). As vaccine durability is key parameter for prolonged protection, mice immunized with RBD g5.1 24mer were monitored out past six months and maintenance of high neutralizing titers was observed (FIG. 8I). As proof-of-concept for expanding this platform to emerging variants, P.1/Gamma RBD g5.1 24mers were engineered (FIG. 13B). Upon BALB/c mice immunization with 2 μg of P. 1/Gamma RBD g5.1 24mer, high binding and cross-neutralization titers were observed (FIG. 13A).

### Single Dose of RBD Nanoparticles Affords Protection in Lethal Challenge Model

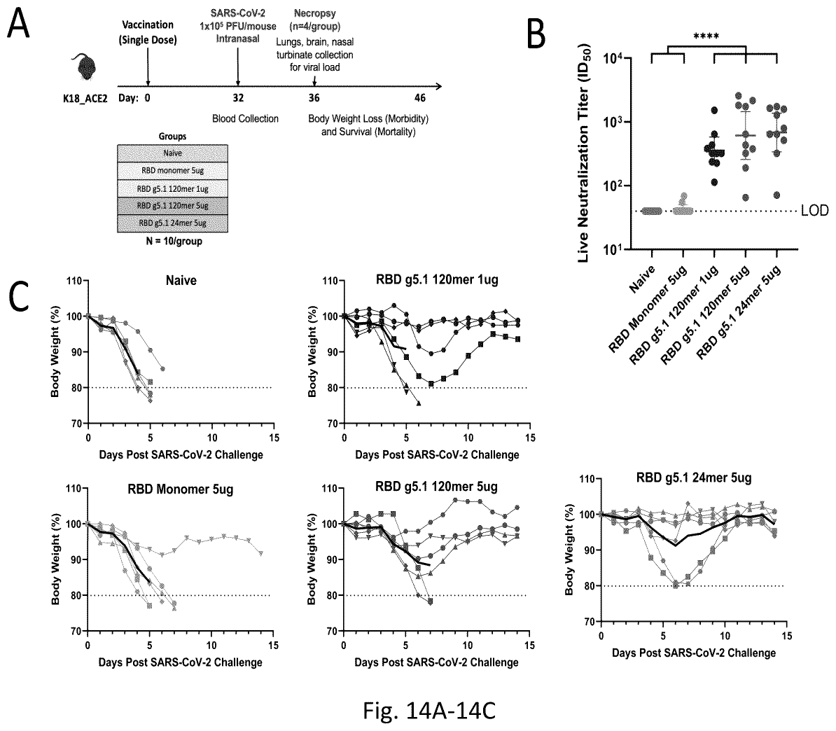

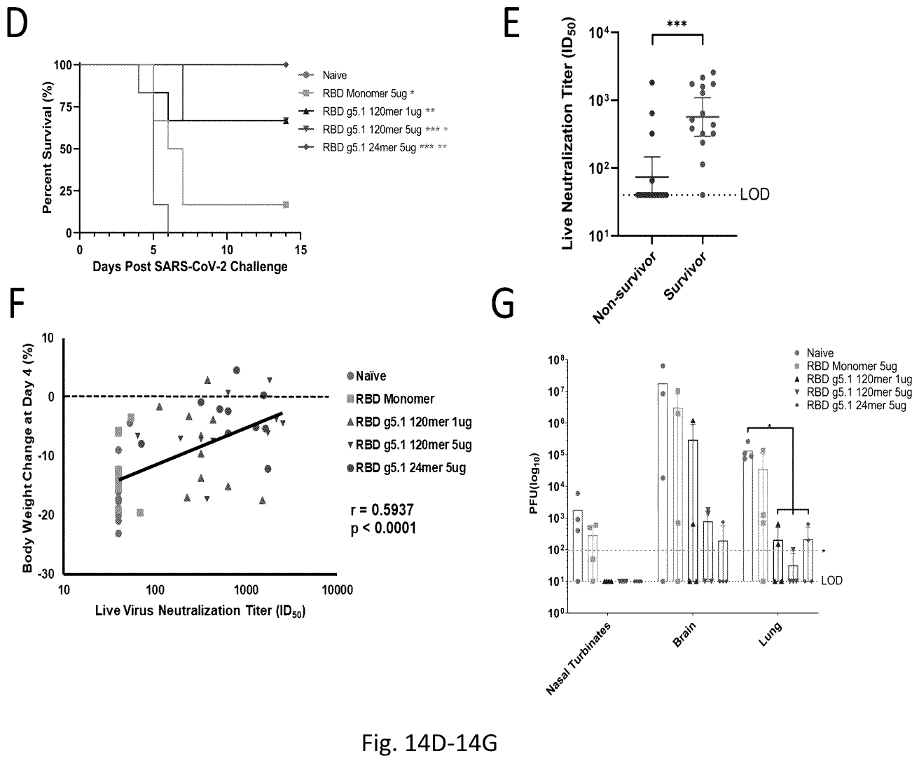

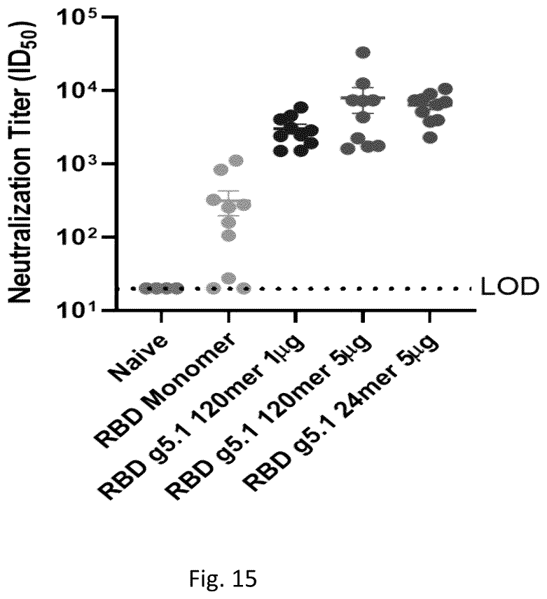

To examine the efficacy of the SARS-COV-2 RBD nanoparticles with rapid seroconversion (RBD g5.1 24mer and RBD g5.1 120mer), a lethal challenge study was pursued (FIG. 14A). B6.Cg-Tg(K18-ACE2)2Prlmn/J(K18-hACE2) mice express human ACE2 on epithelial cells including in the airway (Chow et al., 1997, Proc Natl Acad Sci USA, 94(26): p. 14695-700; McCray et al., 2007, J Virol, 81(2): p. 813-21) and can be infected with SARS-COV-2 resulting in weight loss and lethality (Oladunni et al., 2020, Nat Commun, 11(1): p. 6122) providing a stringent model for testing vaccines (Rathnasinghe et al., 2020, Emerg Microbes Infect, 9(1): p. 2433-2445). Animals were vaccinated with a single shot of 5 μg and 1 μg of the nanovaccines in K18-ACE2 mice representing doses 5- and 25-fold lower than the standard DNA dose (Smith et al., 2020, Nat Commun, 11(1): p. 2601). Prior to a blinded challenge, immunogenicity at day 21 was examined and pseudovirus neutralization titers were observed prior to challenge in all vaccine groups (FIG. 15). Live SARS-COV-2 virus neutralization titers were also observed above the limit of detection for all three nanoparticle groups with mean ID₅₀ of 451, 1028, and 921 for RBD g5.1 120mer 1 μg, 5 μg and RBD g5.1 24mer 5 μg, respectively, compared to a mean ID₅₀ of 29 of RBD monomer (FIG. 14B). The mice were infected with a high dose of SARS-COV-2 1×10⁵ PFU/mouse intranasally and monitored for signs of deteriorating health. Mice immunized with nanovaccines had higher levels of protection from weight loss (FIG. 14C). As expected, the naive group of animals reached 100% morbidity by day 6 and 1/10 animals survived in the RBD monomer group. In both RBD g5.1 120mer groups had 6/10 mice survived the challenge. Strikingly, immunization with RBD g5.1 24mer provided full protection from a lethal SARS-COV-2 challenge (FIG. 14D). All but one animal that survived had a live virus neutralization titer of >100 and 12/15 of the mice that succumbed to infection did not have appreciable neutralization titers (FIG. 14E). A significant correlation was observed between live virus neutralization ID₅₀ titer and body weight loss (FIG. 14F). Viral replication was absent in nasal turbinate which may decrease viral transmissibility, and was reduced in lung tissue and brain tissue for mice immunized with nanovaccines relative to RBD monomer or naïve animals (FIG. 14G). Thus, the DNA-launched nanovaccines can generate potent immunity that provides protection from challenge with a single immunization at a low dose.

### Enhanced Immune Responses to Nanovaccines in Translational Vaccine Models

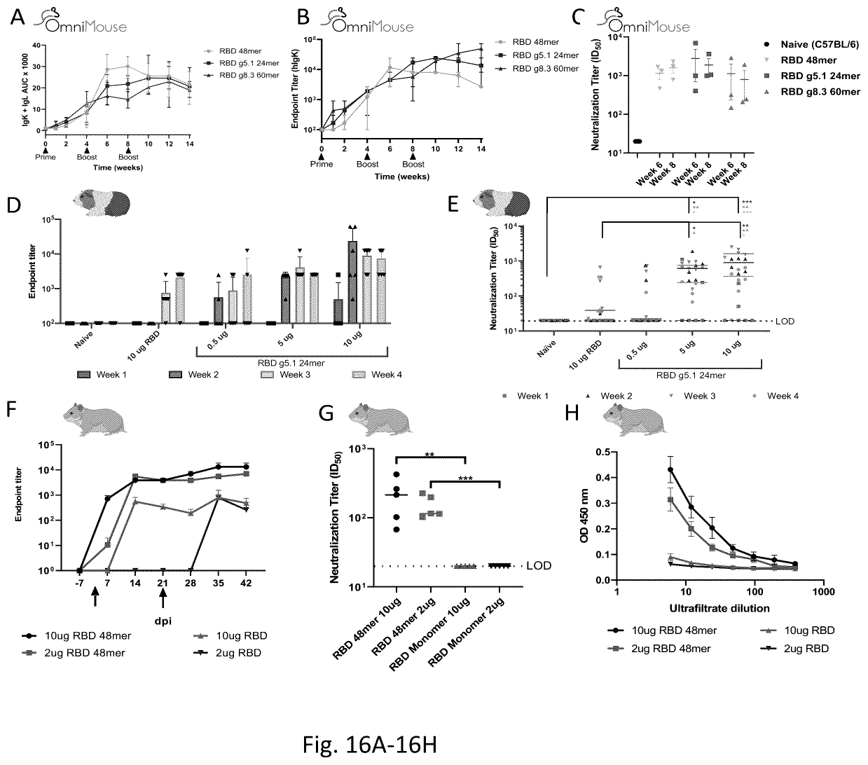

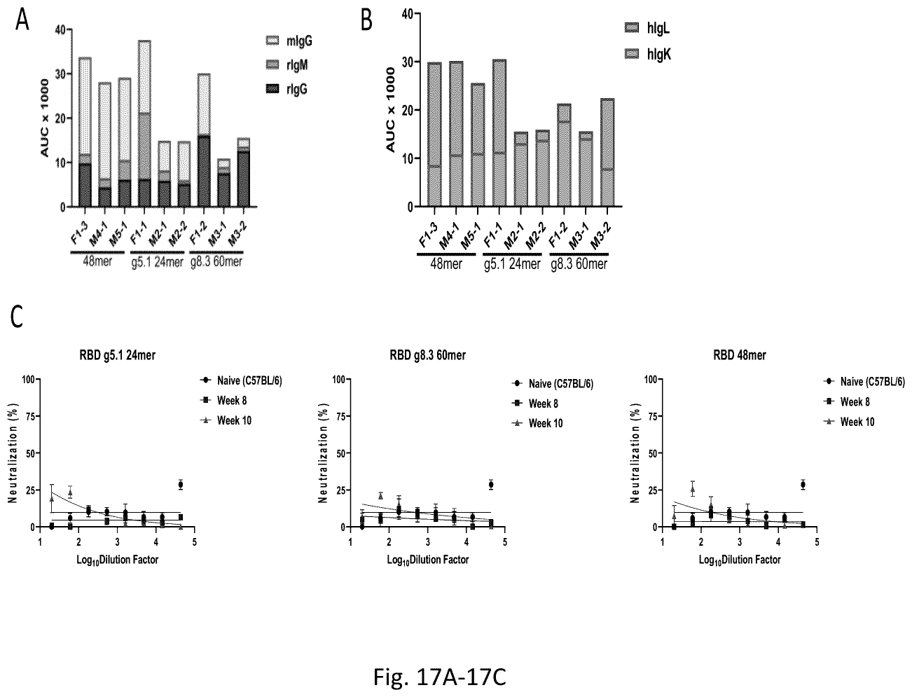

One major challenge for the clinical translation of vaccines is preclinical modeling of human antibody responses to immunogens. OmniMouse® have humanized immunoglobulin loci-transgenic with human V, D and J gene segments (Geurts et al., 2009, Science, 325(5939): p. 433). As a proof-of-concept, OmniMouse® (n=3) were immunized with three different SARS-COV-2 nanoparticle vaccines (RBD 48mer, RBD g5.1 24mer, RBD g8.3 60mer) and increasing RBD-specific human antibodies were measured in serum (FIG. 16A, 16B). Most mice produced high titers of human IgG and a few had robust IgM titers (FIG. 17A, 17B). Potent and specific neutralization was observed in all three groups at weeks 6 and 8 (FIGS. 16C, 17C). Thus, the SARS-CoV-2 nanoparticle platform can be employed in transgenic mice and induce human SARS-COV-2 neutralizing antibodies.

RBD g5.1 24mer was assessed in Hartley guinea pigs (n=6) to examine intradermal vaccine delivery at 0.5, 5 and 10 μg in comparison to RBD monomer at 10 μg. In contrast to the RBD monomer immunized group, full seroconversion of RBD g5.1 24mer was observed in immunized animals at a dose of 5 μg (FIG. 6D). High levels of neutralizing antibodies were obtained in the 10 μg dose group (ID₅₀ of 1840) (FIG. 6E). In a proof-of-concept study of RBD DLNPs prior to the development of RBD g5.1 24mer, Syrian Golden hamsters (n=5) were immunized twice 3 weeks apart with 2 μg and 10 μg of RBD monomer and RBD 48mer (FIG. 6F). The RBD nanoparticle immunized hamsters elicited higher antibody titers after both first and second doses and produced neutralizing antibodies unlike the responses in the RBD monomer vaccine groups (FIG. 6G). To assess the biodistribution of anti-RBD IgG, ultra-filtrated lung lavages were measured and antibodies were found only in the RBD nanoparticle groups (FIG. 6H). In summary, the data presented herein demonstrate that the DLNP vaccines provide enhanced immunogenicity in guinea pigs and hamsters.

### Computational Design of SARS-COV-2 Nanoparticle Immunogens

The data presented herein demonstrate the engineering of a SARS-COV-2 RBD which is immune-focused to neutralizing sites. Immune focusing was verified with single-glycan mass spec. analysis. And it was found that immune-focusing improves ACE2-blocking and neutralizing titers.

The data presented herein demonstrate development of a single gene fusion self-assembling SARS-COV-2 nanoparticle on 7 different scaffolds, which can be delivered in vivo. Further, SARS-COV-2 VOC self-assembling nanoparticles were developed. Engineered RBD nanoparticles elicit cross-neutralization against VOCs and are durable past six months with a single immunization and protection from death in lethal challenge. The nanoparticle vaccine induced human neutralizing abs in human antibody repertoire mouse model and enhanced immunity in guinea pigs and hamsters. DNA-nanos significantly improve immunogenicity of monomeric DNA vaccines. Further, immune-focused RBD nanoparticles are amenable to multiple vaccine platforms.

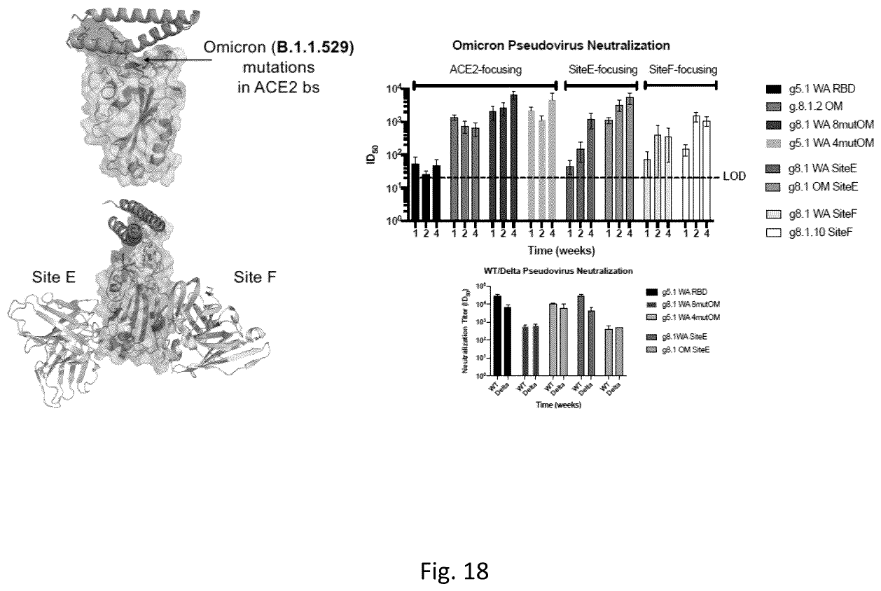

FIG. 18 demonstrates that RBD nanos can be engineered to induce neutralizing antibodies against highly diverse VOCs (Omicron, BA2).

TABLE 1
Sequence Information

| SEQ | Sequence | |
|---|---|---|
| ID NO: | Type | Description |
| 1 | Nucleotide | RBD 48mer |
| 2 | Amino Acid | RBD 48mer |
| 3 | Nucleotide | RBD_g5.1_180mer |
| 4 | Amino Acid | RBD_g5.1_180mer |
| 5 | Nucleotide | RBD_g5.1_120mer (Twist) |
| 6 | Amino Acid | RBD_g5.1_120mer (Twist) |
| 7 | Nucleotide | RBD_g5.1_120mer (Genscript) |
| 8 | Amino Acid | RBD_g5.1_120mer (Genscript) |
| 9 | Nucleotide | RBD_g5.1 60mer |
| 10 | Amino Acid | RBD_g5.1 60mer |
| 11 | Nucleotide | RBD_g5.1 60mer short |
| 12 | Amino Acid | RBD_g5.1 60mer short |
| 13 | Nucleotide | RBD_g5.1 48mer |
| 14 | Amino Acid | RBD_g5.1 48mer |
| 15 | Nucleotide | RBD_g5.1 24mer (Twist) |
| 16 | Amino Acid | RBD_g5.1 24mer (Twist) |
| 17 | Nucleotide | RBD_g5.1 24mer (Genscript) |
| 18 | Amino Acid | RBD_g5.1 24mer (Genscript) |
| 19 | Nucleotide | RBD_g5.1 7mer |
| 20 | Amino Acid | RBD_g5.1 7mer |
| 21 | Nucleotide | RBD_g5.1 14mer trans |
| 22 | Amino Acid | RBD_g5.1 14mer trans |
| 23 | Nucleotide | RBD_g5.1 14mer cys |
| 24 | Amino Acid | RBD_g5.1 14mer cys |
| 25 | Nucleotide | RBD_g8.1_180mer |
| 26 | Amino Acid | RBD_g8.1_180mer |
| 27 | Nucleotide | RBD_g8.1 60mer |
| 28 | Amino Acid | RBD_g8.1 60mer |
| 29 | Nucleotide | RBD_g8.1 24mer |
| 30 | Amino Acid | RBD_g8.1 24mer |
| 31 | Nucleotide | RBD_g8.1 7mer |
| 32 | Amino Acid | RBD_g8.1 7mer |
| 33 | Nucleotide | RBD_g8.2_180mer |
| 34 | Amino Acid | RBD_g8.2_180mer |
| 35 | Nucleotide | RBD_g8.2 60mer |
| 36 | Amino Acid | RBD_g8.2 60mer |
| 37 | Nucleotide | RBD_g8.2 24mer |
| 38 | Amino Acid | RBD_g8.2 24mer |
| 39 | Nucleotide | RBD_g8.2 7mer |
| 40 | Amino Acid | RBD_g8.2 7mer |
| 41 | Nucleotide | RBD_g8.3_180mer |
| 42 | Amino Acid | RBD_g8.3_180mer |
| 43 | Nucleotide | RBD_g8.3 60mer |
| 44 | Amino Acid | RBD_g8.3 60mer |
| 45 | Nucleotide | RBD_g8.3 24mer |
| 46 | Amino Acid | RBD_g8.3 24mer |
| 47 | Nucleotide | RBD_g8.3 7mer |
| 48 | Amino Acid | RBD_g8.3 7mer |
| 49 | Nucleotide | RBD_g5.1 SA 24mer |
| 50 | Amino Acid | RBD_g5.1 SA 24mer |
| 51 | Nucleotide | RBD_g5.1 UK 24mer |
| 52 | Amino Acid | RBD_g5.1 UK 24mer |
| 53 | Nucleotide | RBD_g5.1 BR 24mer |
| 54 | Amino Acid | RBD_g5.1 BR 24mer |
| 55 | Nucleotide | LS3_Omicron_RBD_g5.1_FR_Opt_pVAX |
| 56 | Amino Acid | LS3_Omicron_RBD_g5.1_FR_Opt_pVAX |
| 57 | Nucleotide | LS3_Omicron_KRY_RBD_g5.1_FR |
| 58 | Amino Acid | LS3_Omicron_KRY_RBD_g5.1_FR |
| 59 | Nucleotide | LS3_RBD_g5.2_OC_FR_pVAX |
| 60 | Amino Acid | LS3_RBD_g5.2_OC_FR_pVAX |
| 61 | Nucleotide | LS3_RBD_g5.2b_OC_FR_pVAX |
| 62 | Amino Acid | LS3_RBD_g5.2b_OC_FR_pVAX |
| 63 | Nucleotide | LS3_RBD_g5.2_OCKRY_FR_pVAX |
| 64 | Amino Acid | LS3_RBD_g5.2_OCKRY_FR_pVAX |
| 65 | Nucleotide | LS3_RBD_g5.2b_OCKRY_FR_pVAX |
| 66 | Amino Acid | LS3_RBD_g5.2b_OCKRY_FR_pVAX |
| 67 | Nucleotide | RBD_g5.1_minOM_FR_Opt_pVAX |
| 68 | Amino Acid | RBD_g5.1_minOM_FR_Opt_pVAX |
| 69 | Nucleotide | RBD_g5.1_partialOM_FR_Opt_pVAX |
| 70 | Amino Acid | RBD_g5.1_partialOM_FR_Opt_pVAX |
| 71 | Nucleotide | RBD_g8.1.1_FR_pVAX |
| 72 | Amino Acid | RBD_g8.1.1_FR_pVAX |
| 73 | Nucleotide | RBD_g8.1.2_FR_pVAX |
| 74 | Amino Acid | RBD_g8.1.2_FR_pVAX |
| 75 | Nucleotide | RBD_g8.1.3_FR_pVAX |
| 76 | Amino Acid | RBD_g8.1.3_FR_pVAX |
| 77 | Nucleotide | RBD_g8.1.4_FR_pVAX |
| 78 | Amino Acid | RBD_g8.1.4_FR_pVAX |
| 79 | Nucleotide | RBD_g8.1_partialOM_KO + |
| | | 481_FR_Opt_pVAX |
| 80 | Amino Acid | RBD_g8.1_partialOM_KO + |
| | | 481_FR_Opt_pVAX |
| 81 | Nucleotide | RBD_g8.1.6_FR_pVAX |
| 82 | Amino Acid | RBD_g8.1.6_FR_pVAX |
| 83 | Nucleotide | RBD_g8.1_WA_siteE_FR_Opt_pVAX |
| 84 | Amino Acid | RBD_g8.1_WA_siteE_FR_Opt_pVAX |
| 85 | Nucleotide | RBD_g8.1.8_FR_pVAX |
| 86 | Amino Acid | RBD_g8.1.8_FR_pVAX |
| 87 | Nucleotide | RBD_g8.1_WA_siteF_FR_Opt_pVAX |
| 88 | Amino Acid | RBD_g8.1_WA_siteF_FR_Opt_pVAX |
| 89 | Nucleotide | RBD_g8.1.10_FR_pVAX |
| 90 | Amino Acid | RBD_g8.1.10_FR_pVAX |
| 91 | Nucleotide | RBD_g8.1.1_short_FR_pVAX |
| 92 | Amino Acid | RBD_g8.1.1_short_FR_pVAX |
| 93 | Nucleotide | RBD_g8.1.5_short_FR_pVAX |
| 94 | Amino Acid | RBD_g8.1.5_short_FR_pVAX |
| 95 | Nucleotide | RBD_g8.1.7_short_FR_pVAX |
| 96 | Amino Acid | RBD_g8.1.7_short_FR_pVAX |
| 97 | Nucleotide | RBD_g8.1.9_short_FR_pVAX |
| 98 | Amino Acid | RBD_g8.1.9_short_FR_pVAX |
| 99 | Nucleotide | RBD_g5.1minOM_short_FR_pVAX |
| 100 | Amino Acid | RBD_g5.1minOM_short_FR_pVAX |
| 101 | Nucleotide | LS3_RBD_g5.1_short1_FR_pVAX |
| 102 | Amino Acid | LS3_RBD_g5.1_short1_FR_pVAX |
| 103 | Nucleotide | LS3_RBD_g5.1_short2_FR_pVAX |
| 104 | Amino Acid | LS3_RBD_g5.1_short2_FR_pVAX |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nucleic acid molecule encoding a glycan-coated immunogen modified to encode at least one non-native site for glycan modification.

2. The nucleic acid molecule of claim 1, wherein the nucleotide sequence encoding the immunogen comprises at least two non-native sites for glycan modification.

3. The nucleic acid molecule of claim 1 further comprising at least one selected from the group consisting of an IgE leader sequence and a CD4-helper epitope (LS-3).

4. The nucleic acid molecule of claim 1, comprising at least two tandem repeats of the immunogen.

5. The nucleic acid molecule of claim 1, wherein the immunogen is a SARS Coronavirus 2 (SARS-COV-2) spike protein receptor binding domain (RBD).

6. The nucleic acid molecule of claim 1, wherein the nucleic acid molecule comprises an expression vector.

7. The nucleic acid molecule of claim 1, wherein the nucleic acid molecule is incorporated into a self-assembling nanoparticle.

8. A nanoparticle comprising a nucleic acid molecule encoding a glycan-coated immunogen of claim 1.

9. The nanoparticle of claim 8, wherein the nucleic acid molecule encoding the glycan-coated immunogen is selected from the group consisting of a DNA molecule and an RNA molecule.

10. The nanoparticle of claim 8, wherein the nucleotide sequence encoding the immunogen modified to encode at least one non-native site for glycan modification is operably linked to a nucleotide sequence encoding a nanoparticle scaffold domain.

11. The nanoparticle of claim 10, wherein the nanoparticle scaffold domain is selected from the group consisting of IMX313P, ferritin, lumazine synthase, and PcV.

12. The nanoparticle of claim 10, wherein the nucleic acid molecule comprises a nucleotide sequence selected from the group consisting of:
the nucleotide sequence encodes a peptide comprising an amino acid sequence having at least about 90% identity over an entire length of an amino acid sequence selected from the group consisting of SEQ ID NO:4, SEQ ID NO:6, SEQ ID NO:8, SEQ ID NO: 10, SEQ ID NO:12, SEQ ID NO:14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO: 76, SEQ ID NO: 78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO: 102, and SEQ ID NO: 104; and
the nucleotide sequence encodes a peptide comprising an amino acid sequence selected from the group consisting of SEQ ID NO:4, SEQ ID NO:6, SEQ ID NO:8, SEQ ID NO:10, SEQ ID NO:12, SEQ ID NO:14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO: 70, SEQ ID NO:72, SEQ ID NO: 74, SEQ ID NO: 76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO: 100, SEQ ID NO: 102, and SEQ ID NO: 104.

13. The nanoparticle of claim 12, wherein the nucleic acid molecule comprises a nucleotide sequence selected from the group consisting of:
(a) the nucleotide sequence having at least about 90% identity over an entire length of the nucleic acid sequence selected from the group consisting of SEQ ID NO:3, SEQ ID NO:5, SEQ ID NO: 7, SEQ ID NO:9, SEQ ID NO: 11, SEQ ID NO: 13, SEQ ID NO: 15, SEQ ID NO:17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:74, SEQ ID NO:75, SEQ ID NO:77, SEQ ID NO:79, SEQ ID NO:81, SEQ ID NO:83, SEQ ID NO:85, SEQ ID NO: 87, SEQ ID NO: 89, SEQ ID NO:91, SEQ ID NO:93, SEQ ID NO:95, SEQ ID NO:97, SEQ ID NO:99, SEQ ID NO: 101, and SEQ ID NO: 103; and
the nucleic acid sequence selected from the group consisting of SEQ ID NO:3, SEQ ID NO:5, SEQ ID NO:7, SEQ ID NO:9, SEQ ID NO:11, SEQ ID NO: 13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO: 63, SEQ ID NO:65, SEQ ID NO: 67, SEQ ID NO:69, SEQ ID NO:71, SEQ ID NO:74, SEQ ID NO: 75, SEQ ID NO: 77, SEQ ID NO: 79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO: 101, and SEQ ID NO: 103.

14. An immunogenic composition comprising
(a) at least one nucleic acid molecule of claim 1; or
(b) a nanoparticle comprising a nucleic acid molecule of (a); and
(c) a pharmaceutically acceptable excipient, and an adjuvant.

15.-16. (canceled)

17. A method of inducing an immune response against SARS-COV-2 in a subject in need thereof, the method comprising administering a nucleic acid molecule of claim 1 to the subject.

18. The method of claim 17, wherein administering includes at least one of electroporation and injection.

19. A method of protecting a subject in need thereof from infection with SARS-COV-2, the method comprising administering a nucleic acid molecule of claim 1 wherein the subject is thereby resistant to one or more SARS-COV-2 strains.

20. The method of the claim 19, wherein administering includes at least one of electroporation and injection.

21. A method of treating a subject in need thereof against SARS-COV-2, the method comprising administering a nucleic acid molecule of claim 1.

22. The method of claim 21, wherein administering includes at least one of electroporation and injection.


SEQUENCE LISTING (106 sequences)

SEQ ID NO: 1 - DNA, 1908 bp, Artificial Sequence
  Chemically Synthesized, RBD 48mer
atggactggacttggattctgttcctggtcgcagcagccactcgggtgcatagccgcgtgcagcccactgaaagcattgtgagattccctaacatcaccaatctgtgcccattcggcgaggtgtttaacgccacacggttcgccagcgtgtacgcctggaacaggaagagaatctccaattgcgtggccgactactctgtgctgtataatagcgcctccttctctacctttaagtgctacggcgtgtctcccaccaagctgaacgacctgtgcttcacaaacgtgtacgccgacagctttgtgatcaggggcgatgaggtgagacagatcgcaccaggacagaccggcaagatcgcagactacaactataagctgcccgacgatttcacaggctgcgtgatcgcctggaatagcaacaatctggattccaaagtgggcggcaactacaattatctgtacaggctgttcagaaagagcaacctgaagccctttgagcgggacatctctaccgagatctaccaggccggcagcacaccttgcaacggcgtggagggcttcaattgttactttccactgcagtcttatggcttccagcccacaaacggcgtgggctaccagccttatcgcgtggtggtgctgagctttgagctgctgcacgcaccagcaaccgtgtgcggacctaagaagagcacaaacctggtgaagaataagagggtgcagcctaccgagtccatcgtgagattccccaacatcacaaatctgtgccctttcggcgaagtgtttaacgccacccgctttgcctccgtgtacgcctggaaccggaagcgcatcagtaactgcgtggccgactactccgtgctgtacaacagcgccagcttcagcaccttcaagtgctacggcgtgtccccaactaagctgaacgacctgtgctttaccaacgtgtacgctgactcttttgtgatccggggcgatgaggtgcgccagatcgcacctggccagactggcaagatcgcagactataactacaagctgccagatgatttcacaggatgcgtgattgcttggaattctaacaatctggatagtaaagtgggcggcaattataattacctgtatcggctgttccgcaagtccaacctgaagccatttgagagggatatctccactgaaatctaccaggccggctctacaccctgtaatggagtcgaaggcttcaattgctatttccctctgcagtcctatggcttccagccaaccaacggagtgggataccagccttatagagtcgtggtgctgagctttgagttactgcatgcccctgccaccgtgtgcggacccaagaagtccacaaatctggtcaagaataaggcaagcggggaaagccaggtgcgacagcagttctccaaagacatcgaaaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 2 - protein, 634 aa, Artificial Sequence
  Chemically Synthesized, RBD 48mer
MDWTWILFLVAAATRVHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 3 - DNA, 1815 bp, Artificial Sequence
  Chemically Synthesized, RBD_g5.1_180mer
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaatga

SEQ ID NO: 4 - protein, 603 aa, Artificial Sequence
  Chemically Synthesized, RBD_g5.1_180mer
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 5 - DNA, 1881 bp, Artificial Sequence
  Chemically Synthesized, RBD_g5.1_120mer (Twist)
atggattggacgtggatattgttcctggtagccgctgcgactcgggtccatagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggccgagtccaacccactgagagcatcgtaagatttccgaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccaagaagtctacaaacctcgtaaagaataagcgggtacagccgaccgaatcaatagtccgattccccaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggcctaagaagagcactaatttggtgaagaacaaatagtga

SEQ ID NO: 6 - protein, 625 aa, Artificial Sequence
  Chemically Synthesized, RBD_g5.1_120mer (Twist)
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNK

SEQ ID NO: 7 - DNA, 1881 bp, Artificial Sequence
  Chemically Synthesized, RBD_g5.1_120mer (Genscript)
atggactggacttggattctgtttctggtcgccgctgccactcgcgtgcattctatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcagggtgcagcctaccgagtccatcgtgagattccctaacatcacaaatctgtgcccattcggcgaggtgtttaacgccaccaggtttgccagcgtgtacgcctggaacaggacaagaatctctaattgcgtggccgactacagcgtgctgtataacagcgcctccttctctacctttaagtgctacggcgtgaacccaaccaagctgaatgacctgtgcttcacaaacgtgtacgccgacagctttgtgatcaggggcgatgaggtgagacagatcgcaccaggacagaccggcaagatcgcagactacaactataagctgcccgataatttcacaggctgcgtgatcgcctggaactccaacaatctggactctaaagtgggcggcaactacaattatctgtacaggctgttcagaaagagcaatctgtccccctttgagagggatatcagcaccgagatctaccaggcaggctccaccccttgcaacggaacagagggcttcaattgttactttccactgcagtcttatggcttccagcccacaaacggcgtgggctaccagccttatagagtggtggtgctgagctttgagctgctgcacgcaccagcaaccgtgtgcggacctaagaagtccacaaacctggtgaagaataagcgggtgcagcccaccgagtctatcgtgcgcttccccaatattacaaatctgtgccctttcggcgaagtgtttaacgctactagattcgctagcgtgtacgcttggaaccggacacgcatctccaactgcgtggctgactactctgtgctgtacaatagcgcctccttctctacctttaagtgttacggcgtgaaccctactaagctgaatgacctgtgctttaccaacgtgtacgctgactcctttgtgatccggggcgatgaggtgcgccagatcgcacctggccagactggcaagatcgcagactataactacaagctgccagacaatttcacaggatgcgtgattgcttggaacagcaacaatctggactccaaagtgggcggcaactataattacctgtatcggctgttccgcaagtctaatctgagcccatttgagcgggatatctctactgaaatctaccaggcaggcagcaccccatgtaatggcaccgaaggcttcaattgctactttccactgcagtcctacggatttcagcctaccaatggagtcggctaccagccttatcgcgtcgtggtgctgagctttgagttactgcatgcccctgccaccgtgtgcggacccaagaagtccaccaacctggtcaaaaataagtgataa

SEQ ID NO: 8 - protein, 625 aa, Artificial Sequence; identical to SEQ ID NO: 6
  Chemically Synthesized, RBD_g5.1_120mer (Genscript)

SEQ ID NO: 9 - DNA, 1158 bp, Artificial Sequence
  Chemically Synthesized, RBD g5.1 60mer
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctaatag

SEQ ID NO: 10 - protein, 384 aa, Artificial Sequence
  Chemically Synthesized, RBD g5.1 60mer
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 11 - DNA, 1140 bp, Artificial Sequence
  Chemically Synthesized, RBD g5.1 60mer short
atggattggacgtggatattgttcctggtagccgctgcgactcgggtccatagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggagggagtggaggctcaggaggaggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggaccctagtga

SEQ ID NO: 12 - protein, 378 aa, Artificial Sequence
  Chemically Synthesized, RBD g5.1 60mer short
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 13 - DNA, 1962 bp, Artificial Sequence
  Chemically Synthesized, RBD g5.1 48mer
atggactggacttggattctgttcctggtcgcagcagccactcgggtgcatagcctgaggttcggaatcgtcgcaagtagggctaatcacgctctggtgggcgggtcccgtgtgcagccgacagagagcatcgtgcgcttcccaaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccaagaagagcacgaacctcgtgaagaacaagcgtgtgcagccgacagagagcattgtgcgcttcccaaacatcaccaacctctgcccctttggagaggtctttaatgccaccagatttgcctctgtctatgcctggaaccgcacacgaatcagcaactgtgtggcggactattctgtattgtataacagtgcttcgttctctaccttcaagtgttacggtgtgaaccccaccaagctgaatgacctgtgcttcaccaatgtttatgcagattcctttgtgataagaggagatgaagtccggcaaattgccccagggcagactgggaaaatagcagactacaattacaaactaccagataacttcactggttgtgtcattgcatggaattcaaacaacttggattccaaagtgggcggcaactacaactatctgtacaggcttttcaggaaaagcaatttaagtccttttgaaagagacatctcaactgagatctaccaggctggatcaacaccctgcaatgggacagaaggctttaactgctacttccctctccagtcctatggcttccagcctacaaatggagttggttaccaaccttaccgggtagttgtccttagttttgagctgctgcacgctccagccactgtgtgtggccccaagaagagcacgaatctcgtgaagaacaaggcaagcggggaaagccaggtgcgacagcagttctccaaagacatcgaaaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctagtga

SEQ ID NO: 14 - protein, 652 aa, Artificial Sequence
  Chemically Synthesized, RBD g5.1 48mer
MDWTWILFLVAAATRVHSLRFGIVASRANHALVGGSRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 15 - DNA, 1254 bp, Artificial Sequence
  Chemically Synthesized, RBD g5.1 24mer (Twist)
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 16 - protein, 416 aa, Artificial Sequence
  Chemically Synthesized, RBD g5.1 24mer (Twist)
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 17 - DNA, 1254 bp, Artificial Sequence
  Chemically Synthesized, RBD g5.1 24mer (Genscript)
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcacaaggatcagcaactgcgtggccgattatagcgtgctgtacaattctgccagcttctctacctttaagtgctacggcgtgaaccctaccaagctgaacgatctgtgcttcaccaacgtgtatgccgatagcttcgtgatccggggcgatgaggtgcgccagatcgcccctggccagacaggcaagatcgccgactacaactataagctgccagataatttcaccggctgcgtgatcgcctggaattccaacaatctggactctaaggtgggcggcaactacaactatctgtaccggctgtttcgcaagtccaacctgtctccattcgagagagacatctccacagagatctatcaggccggctccacaccttgtaacggcaccgagggcttcaactgctacttccctctgcagagctatggcttccagcctaccaatggcgtgggctatcagccctaccgggtggtggtgctgtcttttgagctgctgcacgcccctgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 18 - protein, 416 aa, Artificial Sequence; identical to SEQ ID NO: 16
  Chemically Synthesized, RBD g5.1 24mer (Genscript)

SEQ ID NO: 19 - DNA, 966 bp, Artificial Sequence
  Chemically Synthesized, RBD g5.1 7mer
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 20 - protein, 320 aa, Artificial Sequence
  Chemically Synthesized, RBD g5.1 7mer
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 21 - DNA, 1629 bp, Artificial Sequence
  Chemically Synthesized, RBD g5.1 14mer trans
atggattggacgtggatattgttcctggtagccgctgcgactcgggtccatagccgagtccaacccactgagagcatcgtaagatttccgaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccaagaagtctacaaacctcgtaaagaataagcgggtacagccgaccgaatcaatagtccgattccccaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggcctaagaagagcactaatttggtgaagaacaaaggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtagtga

SEQ ID NO: 22 - protein, 541 aa, Artificial Sequence
  Chemically Synthesized, RBD g5.1 14mer trans
MDWTWILFLVAAATRVHSRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKRVQPTESIVRFPNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPKKSTNLVKNKGGSGGSGGSGGSGGGKKQGDADVCGEVAYIQSVVSDCHVPTAELRTLLEIRKLFLEIQKLKVELQGLSKELRFGIVASRANHALV

SEQ ID NO: 23 - DNA, 1602 bp, Artificial Sequence
  Chemically Synthesized, RBD g5.1 14mer cys
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttctccacgcccctgcgaccgtatgtggacccggcggttccggtggatcaggtgggagcggtggatcaggggggggtaagaaacaaggcgacgccgatgtgtgtggggaagttgcctacattcaatctgtcgtcagcgattgtcatgtccccacagccgaattgcgaacccttctcgaaattaggaagctctttcttgaaatccaaaagctcaaggtggaattgcaaggactcagtaaagagcttagatttggcatagtagcgtccagagccaaccatgcccttgtcggtggcagtggcggttccggtggttccggtggcagtggcggtggtaatatcacgaatctgtgtccttttggggaagtgtttaacgctacgcggttcgcatccgtgtacgcctggaatcggacccgaatttccaattgtgtggcagactatagcgtgctttataactctgcttccttttctacctttaaatgctatggggtaaacccaaccaagctcaatgatctgtgtttcaccaatgtgtatgcggatagcttcgtgatccgaggagatgaagttcgacagatcgccccgggacagaccggcaagatagctgattacaactataagttgcccgacaattttacaggctgcgtcattgcatggaactctaataatttggattcaaaagtcggtggaaactataattatctgtacagacttttcaggaagagtaatctgtccccttttgaaagagacatcagcaccgaaatatatcaagcgggaagcaccccttgtaatggcacagagggctttaattgctacttccctttgcaatcttatggttttcagcccaccaacggtgtcggatatcagccctaccgggtagttgtattgtctttcgaactcctgcatgctccagccactgtttgcgggccttaatag

SEQ ID NO: 24 - protein, 532 aa, Artificial Sequence
  Chemically Synthesized, RBD g5.1 14mer cys
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGSGGSGGGKKQGDADVCGEVAYIQSVVSDCHVPTAELRTLLEIRKLFLEIQKLKVELQGLSKELRFGIVASRANHALVGGSGGSGGSGGSGGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGP

SEQ ID NO: 25 - DNA, 1815 bp, Artificial Sequence
  Chemically Synthesized, RBD_g8.1_180mer
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatga

SEQ ID NO: 26 - protein, 603 aa, Artificial Sequence
  Chemically Synthesized, RBD_g8.1_180mer
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGP

SEQ ID NO: 27 - DNA, 1158 bp, Artificial Sequence
  Chemically Synthesized, RBD g8.1 60mer
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatag

SEQ ID NO: 28 - protein, 331 aa, Artificial Sequence
  Chemically Synthesized, RBD g8.1 60mer
DAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGP

SEQ ID NO: 29 - DNA, 1254 bp, Artificial Sequence
  Chemically Synthesized, RBD g8.1 24mer
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctacagtatgtggtcctggcggctccggtggctctggcgggagtggcggatcaggtggtggactgagtaaggacatcatcaaacttctcaacgaacaggtgaacaaggagatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 30 - protein, 416 aa, Artificial Sequence
  Chemically Synthesized, RBD g8.1 24mer
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 31 - DNA, 966 bp, Artificial Sequence
  Chemically Synthesized, RBD g8.1 7mer
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgatgactttacaggttgcgtgatcgcgtggaacagtaataatctggattccaaggttggcgggaattataattacctttatcgcctgtttcggaagtctaacctgagcccgttcgagagagacaactcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggcccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 32 - protein, 320 aa, Artificial Sequence
  Chemically Synthesized, RBD g8.1 7mer
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDDFTGCVIAWNSNNLDSKVGGNYNYLYRLFRKSNLSPFERDNSTEIYQAGSTPCNGTEGFNCYFPLQSYGFQPTNGVGYQPYRVVVLSFENLSAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 33 - DNA, 1815 bp, Artificial Sequence
  Chemically Synthesized, RBD_g8.2_180mer
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatga

SEQ ID NO: 34 - protein, 603 aa, Artificial Sequence
  Chemically Synthesized, RBD_g8.2_180mer
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGP

SEQ ID NO: 35 - DNA, 1158 bp, Artificial Sequence
  Chemically Synthesized, RBD g8.2 60mer
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggccctaatag

SEQ ID NO: 36 - protein, 384 aa, Artificial Sequence
  Chemically Synthesized, RBD g8.2 60mer
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGP

SEQ ID NO: 37 - DNA, 1254 bp, Artificial Sequence
  Chemically Synthesized, RBD g8.2 24mer
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtctgtggacctggtggcagtggcggctctggtggttcaggcggcagtggtggtgggctgtccaaggacataatcaaactgctcaacgaacaggtgaacaaggagatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 38 - protein, 416 aa, Artificial Sequence
  Chemically Synthesized, RBD g8.2 24mer
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 39 - DNA, 966 bp, Artificial Sequence
  Chemically Synthesized, RBD g8.2 7mer
atggattggacttggatcttgttcttggtcgcagcggctactagagtgcacagtttgcgatttggtattgtcgcttcccgcgcaaaccatgcgctcgtgggtggttccggtggcaatattaccaatctttgtcccttcggcgaggtgttcaacgccacgcggttcgcgtctgtatacgcctggaaccgcacccggatcagcaactgtaccgctgactacagcgttctgtacaacagcagcagcttttcaacttttaagtgttacggagtcaaccccacaaaactgaacgacctgtgcttcaccaatgtgtatgcagactcttttgtgatacgcggagatgaagtacgccaaattgcccccggacaaaccggcaagatcgcggactacaactacaagctgcccgataactttacaggttgcgtgatcgcgtggaacagtaataatctggattccaaggttggcgggaattataattacctttatcgcctgtttcggaagtctaacctgagcccgttcgagagagacatctcaacggaaatttatcaagccggcagtaccccttgcaatgggaccgaaggattcaattgttatttcccactccaatcttatggcttccaaccgacaaacggtgttggttatcagccctatcgggtggttgtgctttctttcgaaaaccttagcgctccagctaccgtgtgcgggcccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 40 - protein, 320 aa, Artificial Sequence
  Chemically Synthesized, RBD g8.2 7mer
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFENLSAPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 41 - DNA, 1815 bp, Artificial Sequence
  Chemically Synthesized, RBD_g8.3_180mer
atggactggacctggattctgttcctggtggccgccgccacaagggtgcacagcctctcaattgccccaacgttgatcaaccgggacaagccatacacgaaagaggaacttatggagatattgcggttggccattatagctgaactcgatgcaattaatctctatgaacaaatggcccgctatagcgaagacgaaaatgtgagaaagatcttgttggacgtcgctagggaagagaaagcacacgtaggagagttcatggctttgttgcttaacctcgaccctgagcaagtcacagagctgaagggcgggttcgaggaagttaaagaattgaccggtatagaagctcacattaatgacaacaagaaagaggaaagtaatgtagagtatttcgagaagctcagatctgccttgttggatggagtcaacaagggtcgcagcttgctcaaacatctgcccgttacaagaatagaagggcagtcttttcgagtagacatcatcaaatttgaggacggcgtccgagtggttaaacaagagtataagcctataccccttcttaagaagaagttctacgtcggcattcgagaactgaatgacgggacatatgatgtcagcattgctactaaagccggtgagctgctggttaaagacgaagaaagtcttgtgatccgggaaattttgtcaacggaaggcatcaagaaaatgaaattgtcatcctgggacaatccagaagaagccctgaatgatttgatgaatgcgctccaagaagctagcaatgctagtgctggccccttcggccttattatcaatccaaagcggtacgccaaactgctgaagatctatgaaaagtcaggtaagatgctcgtagaagtactcaaggaaatcttccggggtggaataatcgtaactcttaatatcgacgaaaacaaagtgattatcttcgctaatacgcccgccgttctggacgtggtggtgggtcaagacgttacgctccaggagcttggtccggaaggggatgatgtcgcattcctggtcagtgaagccattggtataagaatcaagaacccggaagctatagttgttctcgaaggcgggtctggtgggagcggtggtagtggtggttctggtggtggtgggtcaggtggcggctcaggcggcggcttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggaccctaatga

SEQ ID NO: 42 - protein, 603 aa, Artificial Sequence
  Chemically Synthesized, RBD_g8.3_180mer
MDWTWILFLVAAATRVHSLSIAPTLINRDKPYTKEELMEILRLAIIAELDAINLYEQMARYSEDENVRKILLDVAREEKAHVGEFMALLLNLDPEQVTELKGGFEEVKELTGIEAHINDNKKEESNVEYFEKLRSALLDGVNKGRSLLKHLPVTRIEGQSFRVDIIKFEDGVRVVKQEYKPIPLLKKKFYVGIRELNDGTYDVSIATKAGELLVKDEESLVIREILSTEGIKKMKLSSWDNPEEALNDLMNALQEASNASAGPFGLIINPKRYAKLLKIYEKSGKMLVEVLKEIFRGGIIVTLNIDENKVIIFANTPAVLDVVVGQDVTLQELGPEGDDVAFLVSEAIGIRIKNPEAIVVLEGGSGGSGGSGGSGGGGSGGGSGGGLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGP

SEQ ID NO: 43 - DNA, 1158 bp, Artificial Sequence
  Chemically Synthesized, RBD g8.3 60mer
atggactggacctggattctgttcctggtggccgccgccacaagggtgcacagcatgcagatctacgaaggaaaactgaccgctgagggactgaggttcggaattgtcgcaagccgcgcgaatcacgcactggtggataggctggtggaaggcgctatcgacgcaattgtccggcacggcgggagagaggaagacatcacactggtgagagtctgcggcagctgggagattcccgtggcagctggagaactggctcgaaaggaggacatcgatgccgtgatcgctattggggtcctgtgccgaggagcaactcccagcttcgactacatcgcctcagaagtgagcaaggggctggctgatctgtccctggagctgaggaaacctatcacttttggcgtgattactgccgacaccctggaacaggcaatcgaggcggccggcacctgccatggaaacaaaggctgggaagcagccctgtgcgctattgagatggcaaatctgttcaaatctctgcgaggaggctccggaggatctggagggagtggaggctcaggaggaggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggaccctaatag

SEQ ID NO: 44 - protein, 384 aa, Artificial Sequence
  Chemically Synthesized, RBD g8.3 60mer
MDWTWILFLVAAATRVHSMQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGP

SEQ ID NO: 45 - DNA, 1254 bp, Artificial Sequence
  Chemically Synthesized, RBD g8.3 24mer
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 46 - protein, 416 aa, Artificial Sequence
  Chemically Synthesized, RBD g8.3 24mer
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 47 - DNA, 966 bp, Artificial Sequence
  Chemically Synthesized, RBD g8.3 7mer
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcaccgctgattactctgttctgtacaattcaagcagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgcagagttacggattccaaccaacaaatggggtgggctaccaaccttatagggtggtcgtcctgtcctttgagcttaaccacagccctgcgaccgtatgtggacccggcgggagcggagggagtggcggatctggcgggagcggcggaggcaaaaaacagggggatgctgatgtctgcggagaggtggcttatatccagagcgtggtgtccgactgccacgtgccaaccgcagagctgcggacactgctggagatcagaaaactgttcctggagattcagaaactgaaagtcgagctgcaggggctgtcaaaagaactgaggttcggaatcgtcgcaagtagggctaatcacgctctggtgtaatag

SEQ ID NO: 48 - protein, 320 aa, Artificial Sequence
  Chemically Synthesized, RBD g8.3 7mer
MDWTWILFLVAAATRVHSLRFGIVASRANHALVGGSGGNITNLCPFGEVFNATRFASVYAWNRTRISNCTADYSVLYNSSSFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELNHSPATVCGPGGSGGSGGSGGSGGGKKQGDADVCGEVAYIQSVVSDCHVPTAELRTLLEIRKLFLEIQKLKVELQGLSKELRFGIVASRANHALV

SEQ ID NO: 49 - DNA, 1254 bp, Artificial Sequence
  Chemically Synthesized, RBD g5.1 SA 24mer
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcacaaggatcagcaactgcgtggccgattatagcgtgctgtacaattctgccagcttctctacctttaagtgctacggcgtgaaccctaccaagctgaacgatctgtgcttcaccaacgtgtatgccgatagcttcgtgatccggggcgatgaggtgcgccagatcgcccctggccagacaggcaacatcgccgactacaactataagctgccagataatttcaccggctgcgtgatcgcctggaattccaacaatctggactctaaggtgggcggcaactacaactatctgtaccggctgtttcgcaagtccaacctgtctccattcgagagagacatctccacagagatctatcaggccggctccacaccttgtaacggcaccaagggcttcaactgctacttccctctgcagagctatggcttccagcctacctacggcgtgggctatcagccctaccgggtggtggtgctgtcttttgagctgctgcacgcccctgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 50 - protein, 416 aa, Artificial Sequence
  Chemically Synthesized, RBD g5.1 SA 24mer
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGNIADYNYKLPDNFTGCVIAWNSNNLDSKVGGNYNYLYRLFRKSNLSPFERDISTEIYQAGSTPCNGTKGFNCYFPLQSYGFQPTYGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 51 - DNA, 1254 bp, Artificial Sequence
  Chemically Synthesized, RBD g5.1 UK 24mer
atggactggacatggatactgtttctggtggccgccgccacaagggtgcactccctgagatttggcatcgtggcctctagggccaaccacgccctggtgggcggctctggcggcaatatcacaaacctgtgcccattcggcgaggtgttcaacgccaccaggtttgcctccgtgtacgcctggaataggaccagaatcagcaactgcgtggccgattattccgtgctgtataactctgcctccttcagcacctttaagtgttacggcgtgaatcctaccaagctgaatgacctgtgcttcaccaacgtgtacgccgacagcttcgtgatcaggggcgatgaggtgcgccagatcgccccaggccagacaggcaagatcgccgactacaattataagctgccagataacttcacaggctgcgtgatcgcctggaacagcaataacctggactccaaggtgggcggcaactataactacctgtatcggctgtttaggaagagcaacctgtctcctttcgagagggatatctccaccgagatctaccaggccggctctacaccatgcaacggcaccaagggcttcaactgctacttccctctgcagtcctatggcttccagcctacctacggcgtgggctaccagccatatagggtggtggtgctgagctttgagctgctgcacgccccagccacagtgtgcggcccaggcggcagcggcggctccggcggcagcggcggctctggcggcggcctgagcaaggacatcatcaagctgctgaacgagcaggtgaacaaggagatgcagtcttccaacctgtacatgtccatgtcctcttggtgttacacccactccctggatggcgccggcctgttcctgtttgatcacgccgccgaggagtatgagcacgccaagaagctgatcatctttctgaacgagaacaacgtgccagtgcagctgacctctatctccgcccctgagcacaagttcgagggcctgacccagatcttccagaaggcctatgagcacgagcagcacatctctgagagcatcaataacatcgtggatcacgccatcaagtccaaggatcacgccacattcaactttctgcagtggtatgtggccgagcagcacgaggaggaggtgctgttcaaggatatcctggacaagatcgagctgatcggcaatgagaaccacggcctgtacctggccgaccagtatgtgaagggcatcgccaagtctaggaagtcctgataa

SEQ ID NO: 52 - protein, 416 aa, Artificial Sequence
  Chemically Synthesized, RBD g5.1 UK 24mer
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTKGFNCYFPLQSYGFQPTYGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 53 - DNA, 1254 bp, Artificial Sequence
  Chemically Synthesized, RBD g5.1 BR 24mer
atggactggacatggatactgtttctggtggccgccgccacaagggtgcactccctgagatttggcatcgtggcctctagggccaaccacgccctggtgggcggctctggcggcaatatcacaaacctgtgcccattcggcgaggtgttcaacgccaccaggtttgcctccgtgtacgcctggaataggaccagaatcagcaactgcgtggccgattattccgtgctgtataactctgcctccttcagcacctttaagtgttacggcgtgaatcctaccaagctgaatgacctgtgcttcaccaacgtgtacgccgacagcttcgtgatcaggggcgatgaggtgcgccagatcgccccaggccagacaggcaccatcgccgactacaattataagctgccagataacttcacaggctgcgtgatcgcctggaacagcaataacctggactccaaggtgggcggcaactataactacctgtatcggctgtttaggaagagcaacctgtctcctttcgagagggatatctccaccgagatctaccaggccggctctacaccatgcaacggcaccaagggcttcaactgctacttccctctgcagtcctatggcttccagcctacctacggcgtgggctaccagccatatagggtggtggtgctgagctttgagctgctgcacgccccagccacagtgtgcggcccaggcggcagcggcggctccggcggcagcggcggctctggcggcggcctgagcaaggacatcatcaagctgctgaacgagcaggtgaacaaggagatgcagtcttccaacctgtacatgtccatgtcctcttggtgttacacccactccctggatggcgccggcctgttcctgtttgatcacgccgccgaggagtatgagcacgccaagaagctgatcatctttctgaacgagaacaacgtgccagtgcagctgacctctatctccgcccctgagcacaagttcgagggcctgacccagatcttccagaaggcctatgagcacgagcagcacatctctgagagcatcaataacatcgtggatcacgccatcaagtccaaggatcacgccacattcaactttctgcagtggtatgtggccgagcagcacgaggaggaggtgctgttcaaggatatcctggacaagatcgagctgatcggcaatgagaaccacggcctgtacctggccgaccagtatgtgaagggcatcgccaagtctaggaagtcctgataa

SEQ ID NO: 54 - protein, 416 aa, Artificial Sequence
  Chemically Synthesized, RBD g5.1 BR 24mer
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGTIADYNYKLPDNFTGCVIAWNSNNLDSKVGGNYNYLYRLFRKSNLSPFERDISTEIYQAGSTPCNGTKGFNCYFPLQSYGFQPTYGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 55 - DNA, 1260 bp, Artificial Sequence
  Chemically Synthesized, LS3_Omicron_RBD_g5.1_FR_Opt_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcgacgaggtgtttaacgccaccaggtttgcctccgtgtacgcctggaaccgcacaaggatcagcaactgcgtggccgattatagcgtgctgtacaatctggcccctttctttacctttaagtgctacggcgtgaaccctaccaagctgaacgatctgtgcttcaccaacgtgtatgccgatagcttcgtgatccggggcgatgaggtgcgccagatcgcccctggccagacaggcaacatcgccgactacaactataagctgccagataatttcaccggctgcgtgatcgcctggaattccaacaagctggactctaaggtgtccggcaactacaactatctgtaccggctgtttcgcaagtccaacctgtctccattcgagagagacatctccacagagatctatcaggccggcaacaagccttgtaacggcaccgcaggcttcaactgctacttccctctgagaagctatagcttccgccctacctatggcgtgggccaccagccctaccgggtggtggtgctgtcttttgagctgctgcacgcccctgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 56 - protein, 418 aa, Artificial Sequence
  Chemically Synthesized, LS3_Omicron_RBD_g5.1_FR_Opt_pVAX
MDWTWILFLVAAATRVHSLRFGIVASRANHALVGGSGGNITNLCPFDEVFNATRFASVYAWNRTRISNCVADYSVLYNLAPFFTFKCYGVNPTKLNDLCFTNVYADSFVIRGDEVRQIAPGQTGNIADYNYKLPDNFTGCVIAWNSNKLDSKVSGNYNYLYRLFRKSNLSPFERDISTEIYQAGNKPCNGTAGFNCYFPLRSYSFRPTYGVGHQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 57 - DNA, 1254 bp, Artificial Sequence
  Chemically Synthesized, LS3_Omicron_KRY_RBD_g5.1_FR
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcacaaggatcagcaactgcgtggccgattatagcgtgctgtacaattctgccagcttctctacctttaagtgctacggcgtgaaccctaccaagctgaacgatctgtgcttcaccaacgtgtatgccgatagcttcgtgatccggggcgatgaggtgcgccagatcgcccctggccagacaggcaagatcgccgactacaactataagctgccagataatttcaccggctgcgtgatcgcctggaattccaacaatctggactctaaggtgggcggcaactacaactatctgtaccggctgtttcgcaagtccaacctgtctccattcgagagagacatctccacagagatctatcaggccggctccacaccttgtaacggcaccgagggcttcaactgctacttccctctgaagagctatggcttccggcctacctacggcgtgggctatcagccctaccgggtggtggtgctgtcttttgagctgctgcacgcccctgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 58 - protein, 416 aa, Artificial Sequence
  Chemically Synthesized, LS3_Omicron_KRY_RBD_g5.1_FR
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLKSYGFRPTYGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 59 - DNA, 1254 bp, Artificial Sequence
  Chemically Synthesized, LS3_RBD_g5.2_OC_FR_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcgatgaggtcttcaatgccacaaggtttgccagtgtatatgcatggaacaggactcgcatctcaaactgcgttgctgattactctgttctgtacaatctggcgcctttctttacattcaagtgttacggagtcaatcctacaaaactgaacgacctctgctttacgaacgtctacgctgactcctttgtcatacggggcgacgaggtgaggcaaattgcgcccggccaaactgggaatattgcggactataattacaaacttcctgataacttcaccggttgtgtgatcgcttggaattcaaacaagctggactccaaggtgtctggcaattacaactacctttataggctctttcggaaaagcaacctgagtcccttcgagcgagatattagtacggagatctaccaggccgggaacaaaccatgcaacggtacggctggtttcaactgttattttccgctgaagagttactccttcaggccaacatatggggtgggccatcaaccttatagggtggtcgtcctgtcctttgagcttctccacgcccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 60 - protein, 416 aa, Artificial Sequence
  Chemically Synthesized, LS3_RBD_g5.2_OC_FR_pVAX
MDWTWILFLVAAATRVHSLRFGIVASRANHALVGGSGGNITNLCPFDEVFNATRFASVYAWNRTRISNCVADYSVLYNLAPFFTFKCYGVNPTKLNDLCFTNVYADSFVIRGDEVRQIAPGQTGNIADYNYKLPDNFTGCVIAWNSNKLDSKVSGNYNYLYRLFRKSNLSPFERDISTEIYQAGNKPCNGTAGFNCYFPLKSYSFRPTYGVGHQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 61 - DNA, 1254 bp, Artificial Sequence
  Chemically Synthesized, LS3_RBD_g5.2b_OC_FR_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcgatgaggtcttcaatgccacaaggtttgccagtgtatatgcatggaacaggactcgcatctcaaactgcgttgctgattactctgttctgtacaatctggcgcctttctttacattcaagtgttacggagtcaatggcacaaaactgaacgacctctgctttacgaacgtctacgctgactcctttgtcatacggggcgacgaggtgaggcaaattgcgcccggccaaactgggaatattgcggactataattacaaacttcctgataacttcaccggttgtgtgatcgcttggaattcaaacaagctggactccaaggtgtctggcaattacaactacctttataggctctttcggaaaagcaacctgagtggcttcgagcgagatattagtacggagatctaccaggccgggaacaaaccatgcaacggtacggctggtttcaactgttattttccgctgaagagttactccttcaggccaacatatggggtgggccatcaaccttatagggtggtcgtcctgtcctttgagcttctccacgcccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 62 - protein, 416 aa, Artificial Sequence
  Chemically Synthesized, LS3_RBD_g5.2b_OC_FR_pVAX
MDWTWILFLVAAATRVHSLRFGIVASRANHALVGGSGGNITNLCPFDEVFNATRFASVYAWNRTRISNCVADYSVLYNLAPFFTFKCYGVNGTKLNDLCFTNVYADSFVIRGDEVRQIAPGQTGNIADYNYKLPDNFTGCVIAWNSNKLDSKVSGNYNYLYRLFRKSNLSGFERDISTEIYQAGNKPCNGTAGFNCYFPLKSYSFRPTYGVGHQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 63 - DNA, 1254 bp, Artificial Sequence
  Chemically Synthesized, LS3_RBD_g5.2_OCKRY_FR_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatcctacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtcccttcgagcgagatattagtacggagatctaccaggccgggtctacgccatgcaacggtacggaaggtttcaactgttattttccgctgaagagttacggattcaggccaacatatggggtgggctaccaaccttatagggtggtcgtcctgtcctttgagcttctccacgcccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 64 - protein, 416 aa, Artificial Sequence; identical to SEQ ID NO: 58
  Chemically Synthesized, LS3_RBD_g5.2_OCKRY_FR_pVAX

SEQ ID NO: 65 - DNA, 1254 bp, Artificial Sequence
  Chemically Synthesized, LS3_RBD_g5.2b_OCKRY_FR_pVAX
atggattggacttggatcttgttcttggtcgcagcggctactagagtgcacagtttgcgatttggtattgtcgcttcccgcgcaaaccatgcgctcgtgggtggttccggtggcaacattaccaacctttgccccttcggcgaggtcttcaatgccacaaggtttgccagtgtatatgcatggaacaggactcgcatctcaaactgcgttgctgattactctgttctgtacaattcagcgagcttctccacattcaagtgttacggagtcaatggcacaaaactgaacgacctctgctttacgaacgtctacgctgactcctttgtcatacggggcgacgaggtgaggcaaattgcgcccggccaaactgggaaaattgcggactataattacaaacttcctgataacttcaccggttgtgtgatcgcttggaattcaaacaacctggactccaaggtgggcggcaattacaactacctttataggctctttcggaaaagcaacctgagtggcttcgagcgagatattagtacggagatctaccaggccgggtctacgccatgcaacggtacggaaggtttcaactgttattttccgctgaagagttacggattcaggccaacatatggggtgggctaccaaccttatagggtggtcgtcctgtcctttgagcttctccacgcccctgcgaccgtatgtggacccggcgggagcggagggagtggcggatctggcgggagcggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 66 - protein, 416 aa, Artificial Sequence
  Chemically Synthesized, LS3_RBD_g5.2b_OCKRY_FR_pVAX
MDWTWILFLVAAATRVHSLRFGIVASRANHALVGGSGGNITNLCPFGEVFNATRFASVYAWNRTRISNCVADYSVLYNSASFSTFKCYGVNGTKLNDLCFTNVYADSFVIRGDEVRQIAPGQTGKIADYNYKLPDNFTGCVIAWNSNNLDSKVGGNYNYLYRLFRKSNLSGFERDISTEIYQAGSTPCNGTEGFNCYFPLKSYGFRPTYGVGYQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 67 - DNA, 1260 bp, Artificial Sequence
  Chemically Synthesized, RBD_g5.1_minOM_FR_Opt_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcacaaggatcagcaactgcgtggccgattatagcgtgctgtacaattctgccagcttctctacctttaagtgctacggcgtgaaccctaccaagctgaacgatctgtgcttcaccaacgtgtatgccgatagcttcgtgatccggggcgatgaggtgcgccagatcgcccctggccagacaggcaagatcgccgactacaactataagctgccagataatttcaccggctgcgtgatcgcctggaattccaacaatctggactctaaggtgggcggcaactacaactatctgtaccggctgtttcgcaagtccaacctgtctccattcgagagagacatctccacagagatctatcaggccggctccacaccttgtaacggcaccgagggcttcaactgctacttccctctgagaagctatggcttccgccctacctatggcgtgggccaccagccctaccgggtggtggtgctgtcttttgagctgctgcacgcccctgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 68 - protein, 418 aa, Artificial Sequence
  Chemically Synthesized, RBD_g5.1_minOM_FR_Opt_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLRSYGFRPTYGVGHQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 69 - DNA, 1260 bp, Artificial Sequence
  Chemically Synthesized, RBD_g5.1_partialOM_FR_Opt_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcacaaggatcagcaactgcgtggccgattatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctggccagacaggcaagatcgccgactacaactataagctgccagataatttcaccggctgcgtgatcgcctggaattccaacaatctggactctaaggtgggcggcaactacaactatctgtaccggctgtttcgcaagtccaacctgtctccattcgagagagacatctccacagagatctatcaggccggcaacaagccttgtaacggcaccgcaggcttcaactgctacttccctctgagaagctatagcttccgccctacctatggcgtgggccaccagccctaccgggtggtggtgctgtcttttgagctgctgcacgcccctgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 70 - protein, 418 aa, Artificial Sequence
  Chemically Synthesized, RBD_g5.1_partialOM_FR_Opt_pVAX
MDWTWILFLVAAATRVHSLRFGIVASRANHALVGGSGGNITNLCPFGEVFNATRFASVYAWNRTRISNCVADYSVLYNSASFSTFKCYGVSPTKLNDLCFTNVYADSFVIRGDEVRQIAPGQTGKIADYNYKLPDNFTGCVIAWNSNNLDSKVGGNYNYLYRLFRKSNLSPFERDISTEIYQAGNKPCNGTAGFNCYFPLRSYSFRPTYGVGHQPYRVVVLSFELLHAPATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 71 - DNA, 1254 bp, Artificial Sequence
  Chemically Synthesized, RBD_g8.1.1_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctaaccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcgtggagggcttcaactgctacttccctctgcagagctatggcttccagcctaccaatggcgtgggctatcagccctaccgggtggtggtgctgtcttttaacaacagcagcgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 72 - protein, 416 aa, Artificial Sequence
  Chemically Synthesized, RBD_g8.1.1_FR_pVAX
MDWTWILFLVAAATRVHSLRFGIVASRANHALVGGSGGNITNLCNFSEVFNATRFASVYAWNRTNISNCVANYSVLYNSASFSTFKCYGVSPTKLNDLCFTNVYADSFVIRGDEVRQIAPNQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 73 - DNA, 1254 bp, Artificial Sequence
  Chemically Synthesized, RBD_g8.1.2_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaatctggcccccttcttcacctttaagtgctacggcgtgagccctaccaagctgaacgatctgtgcttcaccaacgtgtatgccgatagcttcgtgatccggggcgatgaggtgcgccagatcgcccctaaccagacaggcaacatcgccgactacaactataagctgccagatgacttcaccggctgcgtgatcgcctggaattccaacaagctggactctaaggtgagcggcaactacaactatctgtaccggctgtttcgcaagtccaacctgaagccattcgagagagacatctccacagagatctatcaggccggcaacaagccttgtaacggcgtggccggcttcaactgctacttccctctgcggagctatagcttccggcctacctacggcgtgggccaccagccctaccgggtggtggtgctgtcttttaacaacagcagcgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 74 - protein, 416 aa, Artificial Sequence
  Chemically Synthesized, RBD_g8.1.2_FR_pVAX
MDWTWILFLVAAATRVHSLRFGIVASRANHALVGGSGGNITNLCNFSEVFNATRFASVYAWNRTNISNCVANYSVLYNLAPFFTFKCYGVSPTKLNDLCFTNVYADSFVIRGDEVRQIAPNQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 75 - DNA, 1392 bp, Artificial Sequence
  Chemically Synthesized, RBD_g8.1.3_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaatctggcccccttcttcacctttaagtgctacggcgtgagccctaccaagctgaacgatctgtgcttcaccaacgtgtatgccgatagcttcgtgatccggggcgatgaggtgcgccagatcgcccctaaccagacaggcaacatcgccgactacaactataagctgccagatgacttcaccggctgcgtgatcgcctggaattccaacaagctggactctaaggtgagcggcaactacaactatctgtaccggctgtttcgcaagtccaacctgaagccattcgagagagacatctccacagagatctatcaggccggcaacaagccttgtaacggcaccgccggcttcaactgctacttccctctgcggagctataacttcagccctacctacggcgtgggccaccagccctaccgggtggtggtgctgtcttttaacaacagcagcgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataactcgagtctagagggcccgtttaaacccgctgatcagcctcgactgtgccttctagttgccagccatctgttgtttgcccctcccccgtgccttccttgaccctggaaggtgccactcccactgtcctttcctaataa

SEQ ID NO: 76 - protein, 458 aa, Artificial Sequence
  Chemically Synthesized, RBD_g8.1.3_FR_pVAX
MDWTWILFLVAAATRVHSLRFGIVASRANHALVGGSGGNITNLCNFSEVFNATRFASVYAWNRTNISNCVANYSVLYNLAPFFTFKCYGVSPTKLNDLCFTNVYADSFVIRGDEVRQIAPNQTGNIADYNYKLPDDFTGCVIAWNSNKLDSKVSGNYNYLYRLFRKSNLKPFERDISTEIYQAGNKPCNGTAGFNCYFPLRSYNFSPTYGVGHQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSLESRGPVTRSASTVPSSCQPSVVCPSPVPSLTLEGATPTVLS

SEQ ID NO: 77 - DNA, 1254 bp, Artificial Sequence
  Chemically Synthesized, RBD_g8.1.4_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctaaccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcaccgagggcttcaactgctacttccctctgcagagctataacttcagccctaccaatggcgtgggctatcagccctaccgggtggtggtgctgtcttttaacaacagcagcgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 78 - protein, 416 aa, Artificial Sequence
  Chemically Synthesized, RBD_g8.1.4_FR_pVAX
MDWTWILFLVAAATRVHSLRFGIVASRANHALVGGSGGNITNLCNFSEVFNATRFASVYAWNRTNISNCVANYSVLYNSASFSTFKCYGVSPTKLNDLCFTNVYADSFVIRGDEVRQIAPNQTGKIADYNYKLPDDFTGCVIAWNSNNLDSKVGGNYNYLYRLFRKSNLKPFERDISTEIYQAGSTPCNGTEGFNCYFPLQSYNFSPTNGVGYQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 79 - DNA, 1260 bp, Artificial Sequence
  Chemically Synthesized, RBD_g8.1_partialOM_KO+481_FR_Opt_pVAX
atggattggacatggatactgttcctggtggccgccgccaccagagtgcactctctgaggttcggcatcgtggcctccagggccaatcacgccctggtgggcggctccggcggcaacatcaccaatctgtgcaatttctccgaggtgtttaacgccaccaggtttgcctccgtgtacgcctggaaccgcacaaacatcagcaactgcgtggccaactatagcgtgctgtacaatctggcccctttcttcacctttaagtgctacggcgtgtctcctaccaagctgaacgatctgtgcttcaccaacgtgtatgccgatagcttcgtgatccggggcgatgaggtgcgccagatcgcccctaaccagacaggcaacatcgccgactacaactataagctgccagatgacttcaccggctgcgtgatcgcctggaattccaacaagctggactctaaggtgtccggcaactacaactatctgtaccggctgtttcgcaagtccaacctgaagccattcgagagagacatctccacagagatctatcaggccggcaacaagccttgtaacggcaccgccggcttcaactgctacttccctctgcgcagctataatttctcccctaccaatggcgtgggctatcagccctaccgggtggtggtgctgtcttttaataactcttctgccaatgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 80 - protein, 418 aa, Artificial Sequence
  Chemically Synthesized, RBD_g8.1_partialOM_KO+481_FR_Opt_pVAX
MDWTWILFLVAAATRVHSLRFGIVASRANHALVGGSGGNITNLCNFSEVFNATRFASVYAWNRTNISNCVANYSVLYNLAPFFTFKCYGVSPTKLNDLCFTNVYADSFVIRGDEVRQIAPNQTGNIADYNYKLPDDFTGCVIAWNSNKLDSKVSGNYNYLYRLFRKSNLKPFERDISTEIYQAGNKPCNGTAGFNCYFPLRSYNFSPTNGVGYQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 81 - DNA, 1254 bp, Artificial Sequence
  Chemically Synthesized, RBD_g8.1.6_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctaaccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggcaacaagccttgtaacggcgtggccggcttcaactgctacttccctctgcggagctatagcttccggcctacctacggcgtgggccaccagccctaccgggtggtggtgctgtcttttaacaacagcagcgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 82 - protein, 416 aa, Artificial Sequence
  Chemically Synthesized, RBD_g8.1.6_FR_pVAX
MDWTWILFLVAAATRVHSLRFGIVASRANHALVGGSGGNITNLCNFSEVFNATRFASVYAWNRTNISNCVANYSVLYNSASFSTFKCYGVSPTKLNDLCFTNVYADSFVIRGDEVRQIAPNQTGKIADYNYKLPDDFTGCVIAWNSNNLDSKVGGNYNYLYRLFRKSNLKPFERDISTEIYQAGNKPCNGVAGFNCYFPLRSYSFRPTYGVGHQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 83 - DNA, 1260 bp, Artificial Sequence
  Chemically Synthesized, RBD_g8.1_WA_siteE_FR_Opt_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcaagcgcatcagcaactgcgtggccgactatagcgtgctgtacaattctgccagcttctctacctttaagtgctacggcgtgtctcctaccaagctgaacgatctgtgcttcaccaacgtgtatgccgatagcttcgtgatccggggcgatgaggtgcgccagatcgcccctaaccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcaccgagggcttcaactgctacttccctctgcagagctataatttctctcctaccaatggcgtgggctatcagccctaccgggtggtggtgctgtcttttaataactcttctgccaatgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 84 - protein, 418 aa, Artificial Sequence
  Chemically Synthesized, RBD_g8.1_WA_siteE_FR_Opt_pVAX
MDWTWILFLVAAATRVHSLRFGIVASRANHALVGGSGGNITNLCPFGEVFNATRFASVYAWNRKRISNCVADYSVLYNSASFSTFKCYGVSPTKLNDLCFTNVYADSFVIRGDEVRQIAPNQTGKIADYNYKLPDDFTGCVIAWNSNNLDSKVGGNYNYLYRLFRKSNLKPFERDISTEIYQAGSTPCNGTEGFNCYFPLQSYNFSPTNGVGYQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 85 - DNA, 1254 bp, Artificial Sequence
  Chemically Synthesized, RBD_g8.1.8_FR_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcaagaggatcagcaactgcgtggccgattatagcgtgctgtacaatctggcccccttcttcacctttaagtgctacggcgtgagccctaccaagctgaacgatctgtgcttcaccaacgtgtatgccgatagcttcgtgatccggggcgatgaggtgcgccagatcgcccctaaccagacaggcaacatcgccgactacaactataagctgccagatgacttcaccggctgcgtgatcgcctggaattccaacaagctggactctaaggtgagcggcaactacaactatctgtaccggctgtttcgcaagtccaacctgaagccattcgagagagacatctccacagagatctatcaggccggcaacaagccttgtaacggcaccgccggcttcaactgctacttccctctgcggagctataacttcagccctacctacggcgtgggccaccagccctaccgggtggtggtgctgtcttttaacaacagcagcgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 86 - protein, 416 aa, Artificial Sequence
  Chemically Synthesized, RBD_g8.1.8_FR_pVAX
MDWTWILFLVAAATRVHSLRFGIVASRANHALVGGSGGNITNLCPFGEVFNATRFASVYAWNRKRISNCVADYSVLYNLAPFFTFKCYGVSPTKLNDLCFTNVYADSFVIRGDEVRQIAPNQTGNIADYNYKLPDDFTGCVIAWNSNKLDSKVSGNYNYLYRLFRKSNLKPFERDISTEIYQAGNKPCNGTAGFNCYFPLRSYNFSPTYGVGHQPYRVVVLSFNNSSANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 87 - DNA, 1260 bp, Artificial Sequence
  Chemically Synthesized, RBD_g8.1_WA_siteF_FR_Opt_pVAX
atggattggacatggatactgttcctggtggccgccgccaccagagtgcactctctgaggttcggcatcgtggcctccagggccaatcacgccctggtgggcggctccggcggcaacatcaccaatctgtgcaatttctccgaggtgtttaacgccaccaggtttgcctccgtgtacgcctggaaccgcacaaacatcagcaactgcgtggccaactatagcgtgctgtacaattctgccagcttctctacctttaagtgctacggcgtgtctcctaccaagctgaacgatctgtgcttcaccaacgtgtatgccgatagcttcgtgatccggggcgatgaggtgcgccagatcgcccctggccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcaccgagggcttcaactgctacttccctctgcagagctataacttctctcctaccaatggcgtgggctatcagccctaccgggtggtggtgctgtcttttaatctgtctcacgccaatgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 88 - protein, 418 aa, Artificial Sequence
  Chemically Synthesized, RBD_g8.1_WA_siteF_FR_Opt_pVAX
MDWTWILFLVAAATRVHSLRFGIVASRANHALVGGSGGNITNLCNFSEVFNATRFASVYAWNRTNISNCVANYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGTEGFNCYFPLQSYNFSPTNGVGYQPYRVVVLSFNLSHANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 89 - DNA, 1254 bp, Artificial Sequence
  Chemically Synthesized, RBD_g8.1.10_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaatctggcccccttcttcacctttaagtgctacggcgtgagccctaccaagctgaacgatctgtgcttcaccaacgtgtatgccgatagcttcgtgatccggggcgatgaggtgcgccagatcgcccctggccagacaggcaacatcgccgactacaactataagctgccagatgacttcaccggctgcgtgatcgcctggaattccaacaagctggactctaaggtgagcggcaactacaactatctgtaccggctgtttcgcaagtccaacctgaagccattcgagagagacatctccacagagatctatcaggccggcaacaagccttgtaacggcaccgccggcttcaactgctacttccctctgcggagctataacttcagccctacctacggcgtgggccaccagccctaccgggtggtggtgctgtcttttaacctgagccacgccaacgccacagtgtgcggccctggcggctctggcggctccggcggctctggcggcagcggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 90 - protein, 416 aa, Artificial Sequence
  Chemically Synthesized, RBD_g8.1.10_FR_pVAX
MDWTWILFLVAAATRVHSLRFGIVASRANHALVGGSGGNITNLCNFSEVFNATRFASVYAWNRTNISNCVANYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGTAGFNCYFPLRSYNFSPTYGVGHQPYRVVVLSFNLSHANATVCGPGGSGGSGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 91 - DNA, 1218 bp, Artificial Sequence
  Chemically Synthesized, RBD_g8.1.1_short_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctaaccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcgtggagggcttcaactgctacttccctctgcagagctatggcttccagcctaccaatggcgtgggctatcagccctaccgggtggtggtgctgtcttttaacaacagcagcgccaacgccacagtgtgcggccctggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 92 - protein, 404 aa, Artificial Sequence
  Chemically Synthesized, RBD_g8.1.1_short_FR_pVAX
MDWTWILFLVAAATRVHSLRFGIVASRANHALVGGSGGNITNLCNFSEVFNATRFASVYAWNRTNISNCVANYSVLYNSASFSTFKCYGVSPTKLNDLCFTNVYADSFVIRGDEVRQIAPNQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFNNSSANATVCGPGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 93 - DNA, 1218 bp, Artificial Sequence
  Chemically Synthesized, RBD_g8.1.5_short_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaatctggcccccttcttcacctttaagtgctacggcgtgagccctaccaagctgaacgatctgtgcttcaccaacgtgtatgccgatagcttcgtgatccggggcgatgaggtgcgccagatcgcccctaaccagacaggcaacatcgccgactacaactataagctgccagatgacttcaccggctgcgtgatcgcctggaattccaacaagctggactctaaggtgagcggcaactacaactatctgtaccggctgtttcgcaagtccaacctgaagccattcgagagagacatctccacagagatctatcaggccggcaacaagccttgtaacggcaccgccggcttcaactgctacttccctctgcggagctataacttcagccctaccaatggcgtgggctatcagccctaccgggtggtggtgctgtcttttaacaacagcagcgccaacgccacagtgtgcggccctggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 94 - protein, 404 aa, Artificial Sequence
  Chemically Synthesized, RBD_g8.1.5_short_FR_pVAX
MDWTWILFLVAAATRVHSLRFGIVASRANHALVGGSGGNITNLCNFSEVFNATRFASVYAWNRTNISNCVANYSVLYNLAPFFTFKCYGVSPTKLNDLCFTNVYADSFVIRGDEVRQIAPNQTGNIADYNYKLPDDFTGCVIAWNSNKLDSKVSGNYNYLYRLFRKSNLKPFERDISTEIYQAGNKPCNGTAGFNCYFPLRSYNFSPTNGVGYQPYRVVVLSFNNSSANATVCGPGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 95 - DNA, 1218 bp, Artificial Sequence
  Chemically Synthesized, RBD_g8.1.7_short_FR_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcaagaggatcagcaactgcgtggccgattatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctaaccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcaccgagggcttcaactgctacttccctctgcagagctataacttcagccctaccaatggcgtgggctatcagccctaccgggtggtggtgctgtcttttaacaacagcagcgccaacgccacagtgtgcggccctggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 96 - protein, 404 aa, Artificial Sequence
  Chemically Synthesized, RBD_g8.1.7_short_FR_pVAX
MDWTWILFLVAAATRVHSLRFGIVASRANHALVGGSGGNITNLCPFGEVFNATRFASVYAWNRKRISNCVADYSVLYNSASFSTFKCYGVSPTKLNDLCFTNVYADSFVIRGDEVRQIAPNQTGKIADYNYKLPDDFTGCVIAWNSNNLDSKVGGNYNYLYRLFRKSNLKPFERDISTEIYQAGSTPCNGTEGFNCYFPLQSYNFSPTNGVGYQPYRVVVLSFNNSSANATVCGPGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 97 - DNA, 1218 bp, Artificial Sequence
  Chemically Synthesized, RBD_g8.1.9_short_FR_pVAX
atggattggacatggatactgttcctggtggccgccgccaccagagtgcactctctgaggttcggcatcgtggcctccagggccaatcacgccctggtgggcggctccggcggcaacatcaccaatctgtgcaacttcagcgaggtgtttaacgccaccaggtttgcctccgtgtacgcctggaaccgcacaaacatcagcaactgcgtggccaactatagcgtgctgtacaattctgccagcttctctacctttaagtgctacggcgtgagccctaccaagctgaacgatctgtgcttcaccaacgtgtatgccgatagcttcgtgatccggggcgatgaggtgcgccagatcgcccctggccagacaggcaagatcgccgactacaactataagctgccagatgacttcaccggctgcgtgatcgcctggaattccaacaatctggactctaaggtgggcggcaactacaactatctgtaccggctgtttcgcaagtccaacctgaagccattcgagagagacatctccacagagatctatcaggccggctccacaccttgtaacggcaccgagggcttcaactgctacttccctctgcagagctataacttcagccctaccaatggcgtgggctatcagccctaccgggtggtggtgctgtcttttaacctgagccacgccaacgccacagtgtgcggccctggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcatgataa

SEQ ID NO: 98 - protein, 404 aa, Artificial Sequence
  Chemically Synthesized, RBD_g8.1.9_short_FR_pVAX
MDWTWILFLVAAATRVHSLRFGIVASRANHALVGGSGGNITNLCNFSEVFNATRFASVYAWNRTNISNCVANYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGTEGFNCYFPLQSYNFSPTNGVGYQPYRVVVLSFNLSHANATVCGPGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 99 - DNA, 1224 bp, Artificial Sequence
  Chemically Synthesized, RBD_g5.1minOM_short_FR_pVAX
atggattggacatggatactgttcctggtggccgccgccaccagagtgcactctctgaggttcggcatcgtggcctccagggccaatcacgccctggtgggcggctccggcggcaacatcaccaatctgtgccctttcggcgaggtgtttaacgccaccaggtttgcctccgtgtacgcctggaaccgcacaaggatcagcaactgcgtggccgattatagcgtgctgtacaattctgccagcttctctacctttaagtgctacggcgtgaaccctaccaagctgaacgatctgtgcttcaccaacgtgtatgccgatagcttcgtgatccggggcgatgaggtgcgccagatcgcccctggccagacaggcaagatcgccgactacaactataagctgccagataatttcaccggctgcgtgatcgcctggaattccaacaatctggactctaaggtgggcggcaactacaactatctgtaccggctgtttcgcaagtccaacctgtctccattcgagagagacatctccacagagatctatcaggccggctccacaccttgtaacggcaccgagggcttcaactgctacttccctctgagaagctatggcttccgccctacctatggcgtgggccaccagccctaccgggtggtggtgctgtcttttgagctgctgcacgcccctgccacagtgtgcggccctggcggcggcctgagcaaggatatcatcaagctgctgaatgaacaggtcaacaaggaaatgcagagcagcaacctgtacatgtccatgagctcctggtgctatacccactctctggacggagcaggcctgttcctgtttgatcacgccgccgaggagtacgagcacgccaagaagctgatcatcttcctgaatgagaacaatgtgcccgtgcagctgacctctatcagcgcccctgagcacaagttcgagggcctgacacagatctttcagaaggcctacgagcacgagcagcacatctccgagtctatcaacaatatcgtggaccacgccatcaagtccaaggatcacgccacattcaactttctgcagtggtacgtggccgagcagcacgaggaggaggtgctgtttaaggacatcctggataagatcgagctgatcggcaacgagaatcacgggctgtatctggccgaccagtatgtgaagggcatcgctaaaagcaggaaatcaggaagctgataa

SEQ ID NO: 100 - protein, 406 aa, Artificial Sequence
  Chemically Synthesized, RBD_g5.1minOM_short_FR_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLRSYGFRPTYGVGHQPYRVVVLSFELLHAPATVCGPGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 101 - DNA, 1236 bp, Artificial Sequence
  Chemically Synthesized, LS3_RBD_g5.1_short1_FR_pVAX
atggattggacttggatcttgttcttggtcgcagcggctactagagtgcacagtttgcgatttggtattgtcgcttcccgcgcaaaccatgcgctcgtgggtggttccggtggcaacatcaccaacctgtgcccttttggagaagttttcaatgccaccagatttgcttctgtgtatgcctggaacaggactagaatcagcaactgtgtggctgactactctgtgctgtacaactctgcctccttcagcaccttcaagtgctacggcgtgaatcctacaaagctgaatgacctgtgcttcaccaacgtgtatgctgacagctttgtcatcagaggagatgaagtgcggcagatcgcccctggccagacaggcaagattgctgactacaactacaagcttcctgataacttcaccggctgtgtcatcgcctggaacagcaacaacctggacagcaaggtgggcggcaactacaactacctgtacagactcttcaggaagagcaacctgagtccatttgaaagagatatcagcacagagatctaccaggctggcagcacaccctgcaacggtacggagggcttcaactgctacttccctctgcagagctacggcttccagcccaccaatggagtgggctaccagccttacagagtggtggtgctgtcctttgagctgctgcacgcccctgccacagtgtgtggccctggcgggagcggagggagtggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 102 - protein, 410 aa, Artificial Sequence
  Chemically Synthesized, LS3_RBD_g5.1_short1_FR_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGSGGSGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 103 - DNA, 1218 bp, Artificial Sequence
  Chemically Synthesized, LS3_RBD_g5.1_short2_FR_pVAX
atggattggacttggatcttgttcttggtcgcagcggctactagagtgcacagtttgcgatttggtattgtcgcttcccgcgcaaaccatgcgctcgtgggtggttccggtggcaacatcaccaacctgtgcccttttggagaagttttcaatgccaccagatttgcttctgtgtatgcctggaacaggactagaatcagcaactgtgtggctgactactctgtgctgtacaactctgcctccttcagcaccttcaagtgctacggcgtgaatcctacaaagctgaatgacctgtgcttcaccaacgtgtatgctgacagctttgtcatcagaggagatgaagtgcggcagatcgcccctggccagacaggcaagattgctgactacaactacaagcttcctgataacttcaccggctgtgtcatcgcctggaacagcaacaacctggacagcaaggtgggcggcaactacaactacctgtacagactcttcaggaagagcaacctgagtccatttgaaagagatatcagcacagagatctaccaggctggcagcacaccctgcaacggtacggagggcttcaactgctacttccctctgcagagctacggcttccagcccaccaatggagtgggctaccagccttacagagtggtggtgctgtcctttgagctgctgcacgcccctgccacagtgtgtggccctggcggaggcctttcaaaagatattattaagctcctgaatgagcaagtcaataaagaaatgcaatcttctaacctgtacatgagcatgtctagctggtgttatactcacagtctcgacggcgctggcctctttctgttcgatcacgccgctgaagaatatgagcacgcgaagaagcttataatcttcctgaacgagaataatgttcccgtccaactgacgtccatttccgcccctgaacacaagtttgagggtctgactcagatctttcaaaaggcgtacgagcacgagcagcatatcagcgagagcattaacaacattgtcgatcacgccattaaaagcaaagaccacgctaccttcaactttctccaatggtacgtcgccgaacagcatgaggaagaagtattgttcaaggatatactcgataagattgaactgatcggaaatgagaatcacggcctgtacctggccgaccaatacgtcaaaggcattgccaagtctaggaaaagctagtag

SEQ ID NO: 104 - protein, 404 aa, Artificial Sequence
  Chemically Synthesized, LS3_RBD_g5.1_short2_FR_pVAX
MDWTWILFLVAAATRVHSLRFGIVASRANHALVGGSGGNITNLCPFGEVFNATRFASVYAWNRTRISNCVADYSVLYNSASFSTFKCYGVNPTKLNDLCFTNVYADSFVIRGDEVRQIAPGQTGKIADYNYKLPDNFTGCVIAWNSNNLDSKVGGNYNYLYRLFRKSNLSPFERDISTEIYQAGSTPCNGTEGFNCYFPLQSYGFQPTNGVGYQPYRVVVLSFELLHAPATVCGPGGGLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 105 - protein, 18 aa, Artificial Sequence
  Chemically Synthesized, IgE leader
MDWTWILFLVAAATRVHS

SEQ ID NO: 106 - protein, 20 aa, Artificial Sequence
  Chemically Synthesized, LS-3 linker
LRFGIVASRANHALVGGSGG